(19) United States
(12) Patent Application Publication
(10) Pub. No.: US 2026/0306714 A1
(43) Pub. Date: Oct. 8, 2026

(54) VIABLE TISSUE FORMS AND METHODS FOR MAKING AND USING SAME

(71) Applicant: Musculoskeletal Transplant Foundation, Edison, NJ (US)

(72) Inventors: Michael Zbigniew Kubik, Randolph, NJ (US); Brooke Catalfamo, Chesterfield, NJ (US); Amy Kerping Chang-Soo, Honolulu, HI (US); Subha Bhattacharyya, Metuchen, NJ (US); Eric J. Semler, Morganville, NJ (US); Yen-Chen Huang, East Brunswick, NJ (US); Kevin Wu, Morganville, NJ (US); Huy Duc Truong, Middlesex, NJ (US)

(21) Appl. No.: 19/673,485

(22) Filed: May 11, 2026

Related U.S. Application Data
(62) Division of application No. 17/784,133, filed on Dec. 17, 2020, now abandoned.
(60) Provisional application No. 62/948,905, filed on Dec. 17, 2019.
(60) Provisional application No. 63/316,764, filed on Mar. 4, 2022.

(51) Int. Cl. A61L 27/36

(52) U.S. Cl. CPC A61L 27/3608; A61L 27/3612; A61L 27/3691

(57) ABSTRACT

Preserved tissue forms are described which include a lyopreserved tissue sample containing endogenous viable cells and which retain or promote biological activity even after storage at temperatures above freezing. In some embodiments, the lyopreserved tissue sample comprises bone tissue. Methods of use for the lyopreserved bone tissue forms include repairing, reconstructing, otherwise treating a bone defect, such as by rehydrating the lyopreserved bone tissue form and placing it in a subject proximate the bone defect. Methods of use include orthopedic surgery which may involve intervertebral spinal fusion, posterolateral spinal fusion, and other bone fusion treatments. Kits are also described which include the lyopreserved bone tissue sample contained in a first container comprising a moisture barrier, and instructions. The kit may also include storage media, one or more biocompatible fluids, one or more additional tissue matrices and materials, as well as additional containers to contain such additional components.

### CROSS-REFERENCE TO RELATED APPLICATIONS

The present application is a divisional of U.S. patent application Ser. No. 17/784,133, filed Jun. 10, 2022, now allowed and which is a national phase of International Patent Application No. PCT/US2020/065688, filed Dec. 17, 2020, published as WO2021/127230 and which claims the benefit of U.S. Provisional Application No. 62/948,905, filed on Dec. 17, 2019, and the present application also claims the benefit of U.S. Provisional Patent Application No. 63/316,764, filed Mar. 4, 2022, the entire disclosures of all of which are hereby incorporated by reference herein.

### FIELD OF THE INVENTION

The present invention relates generally to preserved tissue forms which contain endogenous viable cells and retain or promote biological activity after being stored at temperatures above freezing for extended periods of time. The present invention also relates to methods for making and using such preserved tissue forms.

### BACKGROUND OF THE INVENTION

Various tissue forms which are derived from processed tissue samples recovered from donors (either living or deceased) are known and useful as grafts for tissue repair and reconstruction in recipients having tissue which is damaged, diseased, atrophied or which could otherwise benefit from such treatment. In some cases, it is beneficial for the tissue form to retain or promote biological activity which facilitates tissue healing, growth and/or reconstruction. Such biological activity may be provided by endogenous or exogenous growth factors, proteins, viable cells, or other biologically active substances present in the tissue form. Similarly, viable and non-viable cells and their products and components may create, participate in, or facilitate biologic signaling processes and networks which control or manage biological activity or processes.

Viable cells often produce or recruit biologically active substances such as growth factors and proteins. However, many processing techniques for producing tissue forms from recovered tissue samples tend to devitalize cells such that they no longer produce or recruit such biologically active substances. Even when the processing techniques do not devitalize cells present in the tissue samples, storage of the resulting tissue forms at room temperature, or temperatures above freezing, often results in the loss of a portion or all of the viable cells. Accordingly, much effort has been spent developing processing and preservation techniques for producing tissue forms containing viable endogenous cells and for storing those tissue forms under conditions which preserve the viability of the cells, as well as their biologic activity and ability to produce signaling factors and participate in biologic signaling processes.

In some cases, recovered tissue samples or the tissue forms produced therefrom are simply frozen by exposure to temperatures at or below about 0° C., which is known to devitalize cells. In other cases, recovered tissue samples or the tissue forms produced therefrom are cryopreserved by contacting them with one or more cryopreservation agents and exposing the tissue samples or tissue forms and cryopreservation agent(s) to temperatures at or below about 0° C. Cryopreserving is known to preserve a portion of the viable cells present in the tissue sample or tissue form, but requires that the resulting tissue form be stored at temperatures at or below freezing, often at temperatures of about −30° C. or less, or even about −80° C. or less, for long term viability. Cryopreserved tissue forms must also be thawed prior to implantation as grafts.

In still other cases, recovered tissue samples or the tissue forms produced therefrom are preserved by lyophilizing (i.e., freeze-drying) by contacting them with one or more lyophilizing agents and freezing them (either controlled rate or not), followed by drying under vacuum. Lyophilization is traditionally known to devitalize cells, but when viable tissues are lyophilized under appropriate conditions, it may be possible for these tissues to still retain viable cells as well as biologically active substances, such as growth factors or proteins. Lyophilized tissue forms can be stored for extended periods of time above freezing temperatures (greater than about 0° C.), but typically require rehydration prior to implantation as grafts.

It would be helpful to develop processing techniques that produce preserved tissue forms containing viable cells endogenous to the initial recovered tissue samples from which the tissue forms are prepared, where the preserved tissue forms can be stored above freezing for extended periods of time (for example, from at least 14 days to 365 days, or at least 1 year, 2 years, 3 years, or even more). Such extended storage of viable tissue forms above freezing temperatures would also facilitate transport or shipping of the viable tissue forms over longer distances and times than currently possible in some cases. The present invention provides a combination of particular protectants and lyopreserving techniques which produce viable tissue forms that can be stored for extended time periods above freezing of from at least 14 days to 365 days, or at least 1 year, 2 years, 3 years, or even more, while retaining a population of viable endogenous cells.

### SUMMARY OF THE INVENTION

The present invention relates to preserved tissue forms which contain endogenous viable cells and retain or promote biological activity after being stored at temperatures above freezing for extended periods of time. The present invention also relates to methods for making and using such preserved tissue forms.

In one embodiment, a preserved tissue form for implanting in or on a subject is provided which comprises a preserved tissue sample which is derived from a recovered tissue sample and contains a post-lyopreservation population of endogenous viable cells which is a portion of a pre-lyopreservation population of endogenous viable cells of the recovered tissue sample.

In another embodiment, a preserved tissue form for implanting in or on a subject is provided which comprises a preserved tissue sample which is derived from a recovered tissue sample and contains a post-lyopreservation population of endogenous viable cells which is a portion of a pre-contact population of endogenous viable cells of the recovered tissue sample.

In another embodiment, a preserved tissue form for implanting in or on a subject is provided which comprises a preserved tissue sample which is derived from a recovered tissue sample and is capable of storage at a temperature above freezing for an extended period of time, after which the preserved tissue sample contains a retained population of endogenous viable cells which is a portion of a post-lyopreservation population of endogenous viable cells. In some embodiments, the extended period of time is from 14 days to 5 years.

In still another embodiment, a preserved tissue form for implanting in or on a subject is provided which comprises a preserved tissue sample comprising a tissue type, wherein, after storage at a temperature above freezing for an extended period of time, the preserved tissue sample contains a post-lyopreservation population of endogenous viable cells, wherein the post-lyopreservation population of endogenous viable cells of the preserved tissue sample is substantially comparable to a post-cryopreservation population of endogenous viable cells of a cryopreserved tissue sample which comprises the same tissue type as the preserved tissue sample. In some embodiments, the extended period of time is from 14 days to 5 years. In some embodiments, the post-lyopreservation population of endogenous viable cells of the preserved tissue sample is ±90% of the post-cryopreservation population of endogenous viable cells of the cryopreserved tissue sample.

The preserved tissue form of may further comprise one or more biocompatible fluids, wherein the lyopreserved tissue sample is rehydrated by contact with the one or more biocompatible fluids. After the preserved tissue sample is rehydrated and implanted in or on a subject at an implantation site, the preserved tissue form retains beneficial biological activity, promotes beneficial biological activity, or both. The beneficial biological activity comprises promoting, at the implantation site, at least one of: tissue healing, tissue growth, and tissue generation.

A method is also provided for preparing a preserved tissue sample, which comprises the steps of:
(A) recovering a tissue sample from a donor;
(B) optionally, cleaning the tissue sample;
(C) optionally, disinfecting the tissue sample;
(D) optionally, modifying one or more of the size, shape and other physical characteristics of the tissue sample by applying one or more physical treatments, chemical treatments, or combinations thereof;
(E) contacting the tissue sample with one or more protectants for a period of contacting time, to form a tissue-protectant mixture comprising a quantity of tissue sample and one or more protectants;
(F) optionally, prior to lyopreserving, storing the tissue-protectant mixture, for a period of storage time, at a storage temperature (e.g., less than −80° C., or less than −50° C.);
(G) optionally, during or after the step of (E) contacting the tissue sample with one or more protectants and prior to lyopreserving, incubating the tissue-protectant mixture at an incubation temperature, for a period of incubation time; and
(H) lyopreserving the tissue-protectant mixture by first freezing the tissue-protectant mixture, and then drying the frozen tissue-protectant mixture (optionally under vacuum) to produce a preserved tissue sample having a post-lyopreservation population of endogenous viable cells,
   wherein the preserved tissue sample is capable of storage at a temperature above freezing for an extended period of time after which the preserved tissue sample contains a retained population of endogenous viable cells which is a portion of the post-lyopreservation population of endogenous viable cells. In some embodiments, wherein the extended period of time is from 14 days to 5 years, such as from 14 to 150 days.

The one or more protectants are selected from the group consisting of: sugars, polyphenols, carotenoids, and combinations thereof. In some embodiments, the one or more protectants comprises: glucose, fructose, sucrose, trehalose, dextran, EGCG, and combinations thereof. In some embodiments, the contacting step (E) comprises contacting the tissue sample with a protectant solution comprising the one or more protectants and at least one biologically compatible fluid.

In some embodiments, the incubation temperature, at which the step of (G) incubating the tissue-protectant mixture prior to lyopreserving is performed, is selected from a room temperature, a refrigerating temperature, a warming temperature, and combinations thereof. In some embodiments, the incubation temperature is a refrigerating temperature comprising from about 2° C. to about 8° C. The period of incubation time, for which the step of (G) incubating the tissue-protectant mixture prior to lyopreserving is performed, may be from greater than zero seconds to about 48 hours.

The tissue sample recovered from a donor comprises a tissue type selected from: adipose, amnion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, placental, skin, spleen, stomach, synovial, tendon, testes, umbilical cord, urological, vascular, vein, and a combination thereof.

### BRIEF DESCRIPTION OF THE DRAWINGS

The present invention will be further explained with reference to the attached figure, in which:

FIG. 1 is a graph of the average ATP values obtained by ATP Assay testing performed on preserved cancellous bone tissue samples to confirm cell viability at 0, 4, 8, 10 and 12 weeks;

FIG. 2 provides stain images for specific compounds found in lyopreserved bone grafts; and

FIG. 3 provides SEM images of the structure of lyopreserved bone grafts at several magnifications;

FIGS. 4A and 4B provide higher magnification SEM images of the structure of lyopreserved bone grafts, showing the inherent D-spacing of collagen therein, as identified by ~67 nm periodic band spacing;

FIG. 5 provides images of pH and relative color for different base solutions used to mix trehalose and EGCG for processing of lyopreserved grafts;

FIG. 6 is a graph of the average % proliferation of peripheral blood mononuclear cells in response to lyopreserved grafts and control groups as part of a two-war mixed lymphocyte reaction (MLR) assay;

FIG. 7 and provides the layout for the indirect contact seeding experiments for macrophages in presence of lyophilized grafts and other test groups;

FIG. 8 is a graph of the average pro-healing, M2 polarization marker CD206 obtained by Mannose ELISA from macrophage culture media extracts after culturing in presence of lyopreserved tissue and other test groups at Day 1, 3, 5 and 7; and

FIG. 9 provides representative images of lyopreserved grafts having pre-formed shapes and containing different ratios of cortical fibers and milled cancellous granules.

### DETAILED DESCRIPTION OF THE INVENTION

Detailed descriptions of one or more embodiments of the present invention are disclosed herein. It should be understood that the disclosed embodiments are merely illustrative of the invention which may be embodied in various forms. In addition, each of the examples given in connection with the various embodiments of the invention is intended to be illustrative, and not restrictive. Further, the figures are not necessarily to scale, and some features may be exaggerated to show details of particular components. Measurements, specifications and the like shown in the figures are intended to be illustrative, and not restrictive. Therefore, specific structural and functional details disclosed herein are not to be interpreted as limiting, but merely as examples for teaching one skilled in the art to variously employ the present invention.

The articles “a” and “an” are used herein to refer to one or to more than one (i.e., at least one) of the grammatical object of the article. By way of example, “an element” means one element or more than one element.

For convenience, certain terms employed herein are enumerated below. Unless defined otherwise, all technical and scientific terms used herein have the same meaning as commonly understood by one of ordinary skill in the art to which this disclosure belongs. The following definitions are intended to enable persons of ordinary skill in the relevant art to understand, make and use the inventions described herein without undue experimentation, however, the definitions should not be construed to unreasonably limit the meaning or scope of the terms.

As used herein, the term “about” means within 20%, more preferably within 10% and most preferably within 5%. The term “substantial” means more than 50%, such as more than 60%, or more than 70%, preferably more than 80% or 85%, and most preferably more than 90%, or 95%, or even 98%.

The term “substantially comparable” means within ±90%, such as within ±85%, or within ±80%, or within ±75%, or within ±70%, or within ±65%, or within ±60%, or within ±55%, or within ±50%, or within ±45%, or within ±40%, or within ±35%, or within ±30%, or within ±25%, or within ±20%, or within ±15%, preferably within ±10%, and most preferably within ±5%, including within ±any percent between 90% and 1%.

As used herein, “basal media” includes any medium which maintains the viability and/or supports the growth of cells (whether isolated, cultured, resident in tissue, or otherwise positioned or adhered on a substrate) and typically comprises a carbon source (e.g., a simple sugar, such as glucose, etc.), water, various salts (such as calcium chloride, potassium chloride, magnesium sulfate, sodium chloride, etc.), amino acids, and optionally vitamins (such as thiamine, riboflavin, folic acid, etc.). Suitable basal media include, without limitation, minimal essential medium (MEM), basal medium Eagle (BME), Dulbecco's Modified Eagle's Medium (DMEM), etc.

“Biologically compatible fluid” and “biocompatible fluid” are used interchangeably herein and mean a fluid for combination with a protectant which will not produce a toxic, injurious, or immunologic response when contacted with living tissue. Examples of biologically compatible fluids suitable for combination with one or more protectants include, without limitation: an aqueous buffer (for example without limitation, phosphate buffered saline (“PBS”)), a buffered or non-buffered isotonic solution (for example without limitation, an aqueous sodium chloride solution (e.g., from about 0.1 weight % (wt %) to about 1 wt %, such as about 0.9 wt %), a lactated Ringer's solution, a cell storage or culture media (for example without limitation, DMEM, a basal medium (e.g., Basal Medium Eagle (BME), or other similar media)), platelet rich plasma (PRP), lecithin, alginate, hyaluronic acid (HA), a derivative or salt of HA (e.g., sodium hyaluronate), bone marrow, other suitable biologically compatible fluids known to persons of ordinary skill in the relevant art, and mixtures thereof.

“Cryopreservation,” as used herein, means a preservation technique which avoids formation of ice crystals inside cells (whether isolated or contained in a tissue sample) during freezing by first contacting the cells, or tissue sample containing cells, with one or more cryopreservation agents which replace the water inside the cells, followed by freezing the tissue sample by reducing the temperature to 0° C. or below. Thus, cryopreservation methods generally involve contacting a tissue sample recovered from a donor with one or more cryopreservation agents which means that the tissue sample contains a pre-contact population of endogenous viable cells (i.e., measured after performing all desired processing steps and prior to contacting with the cryopreservation agent(s)), and a post-contact (or pre-cryopreservation) population of endogenous viable cells (i.e., measured after performing all desired processing steps and contacting the tissue sample with one or more cryopreservation agents, but prior to cryopreserving the processed tissue sample). A cryopreserved tissue sample contains a preserved TO (or “Week 0,” “post-cryopreservation”) population of endogenous viable cells. Depending on if and how long after cryopreservation the cryopreserved tissue sample is stored (at well below freezing temperatures, as necessary to maintain viability of the endogenous cells), a cryopreserved tissue sample will also contain a retained population of endogenous viable cells as measured after a period of storage time such as, without limitation, at least 14 days, or least 28 days, or at least 56 days, or at least 70 days, at least 90 days, at least 180 days, at least 365 days, at least 1 year, 2 years, 3 years, or any point in between, or even longer.

“Cryopreservation agents,” as used herein means intracellular cryoprotectants which operate by permeating the cell membrane and replacing the intracellular water to reduce the intracellular water concentration and, thereby, reduce the amount of ice formed within the cell during freezing. Cryopreservation agents include, without limitation, glycols (alcohols containing at least two hydroxyl groups), such as ethylene glycol, propylene glycol, glycerol, and propanediol, as well as other compounds such as formamide, dimethylsulfoxide (DMSO), methanol, dimethyl acetamide, dimethyl formamide. It is known that intracellular cryoprotectants are often harmful and devitalize cells during performance of cryopreservation.

“Disinfection” and “disinfecting” as used herein, in all of their grammatical forms, is any process that reduces or minimizes bioburden (e.g., bacteria, fungi, etc.), viral load, or both, of an object (e.g., a tissue, a container for tissue, or an implement for processing tissue) and typically, but without limitation, involves contacting, rinsing or soaking the object with one or more disinfecting agents or a solution containing one or more disinfecting agents.

“Disinfecting agents” include, any substances, molecules or other materials which accomplish disinfection including, without limitation, chlorine and chlorine compounds (e.g., hydrochloric acid), formaldehyde, glutaraldehyde, alcohols (e.g., ethanol, isopropyl alcohol), peracetic acid, surfactants (e.g., Triton X-100, Tween, Sodium Dodecyl Sulfate) combinations thereof, and solutions containing same.

The term “dormant” is used herein to describe tissue and cells that are in a state in which they have little to no metabolic activity, but such tissue or cells have retained the ability to be returned to a state in which evidence of metabolic activity can be detected. “Dormant” cells may be reinvigorated using methods such as by rehydrating with a biologically compatible fluid. For example, without limitation, tissue and cells may be described as “dormant” after being subjected to a preservation method and during subsequent storage for a period of time, as described herein.

“Endogenous” as used herein refers to that which is naturally occurring, incorporated within, housed within, adherent to, attached to or resident in.

“Exogenous” as used herein, refers to that which is not originally naturally present and, therefore, is introduced from or produced outside, an organism, cell, tissue, system, graft or implant. Exogenous cells, materials or substances may be derived from the same or a different individual as is intended to receive the cells, materials, substances, tissue, system, graft or implant.

“Freeze” and “freezing,” as used herein, refers to a preservation technique which involves cooling a tissue sample, with or without a preservative or preservative solution which may be present, to a temperature below the freezing temperature of the tissue sample or of the tissue sample and preservative or preservative solution and results in the formation of ice crystals within the frozen tissue sample, and sometimes within cells contained therein. As will be readily recognized by persons of ordinary skill in the relevant art, the aforesaid freezing temperature may be about 0° C., or it may be less than or greater than about 0° C., depending on the type of tissue sample and the type of preservative or preservative solution, if present. Before use, a preserved tissue which has been frozen must be reconstituted by thawing.

“Freeze-drying” is synonymous and used interchangeably herein with the terms “lyophilization” and “lyophilizing” (see below).

A “graft” as used herein means a tissue or organ used for transplantation to, or other placement in or on, a subject (e.g., a patient). Some grafts are made from or otherwise include tissue matrices produced by processing tissue samples recovered from one or more donors. The donor(s) and the receiving subject may each, independently, be: a mammal (including humans and non-human mammals), a reptile, an amphibian, fish, and/or a bird. Furthermore, “grafts” include, but are not limited to, a self-tissue transferred from one body site to another in the same individual (“autologous graft”), a tissue transferred between genetically different members of the same species (“allograft”), and a tissue transferred between different species (“xenograft”).

“Lyophilization” and “lyophilizing” (also referred to as “freeze-drying”), as used herein refers to a preservation technique which involves freezing a tissue sample (either via controlled rate or not), followed by drying under vacuum, which removes ice and other frozen solvents from the frozen tissue sample through the process of sublimation and removes bound water molecules through the process of desorption. The drying phase of a lyophilizing process is typically performed in two steps which include a primary drying step which removes the majority of the ice and frozen solvent (e.g., at least about 70 wt %, and often at least about 90 wt %), followed by a secondary drying step which removes additional ice and frozen solvent from the frozen tissue sample to produce a lyophilized preserved tissue having less than about 10 wt %, such as less than about 6 wt %, or even less than about 1 wt % water.

“Lyophilization agents” as used herein are extracellular lyoprotectants that do not permeate the cell membrane and include: monosaccharides (e.g., glucose, sucrose), disaccharides (e.g., trehalose, lactose) and polysaccharides (e.g., dextran, hydroxyethyl starch (HES), cellulose), flavonoid polyphenols (e.g., catechins such as, without limitation, epigallocatechin gallate (EGCG), sugar alcohols (e.g., mannitol, sorbitol, xylitol, erythritol, adonitol, etc.). Combinations of these are also effective lyophilization agents.

“Lyopreserving” and “lyopreservation” as used herein refer to a preservation technique which involves freezing (either via controlled rate or not) a tissue sample in the presence of one or more specific protectants or a protectant solution containing them as described herein, followed by drying under vacuum which may be performed in a single step at a constant temperature or at temperatures varied within a single range, or in two steps including a primary drying step at temperatures within a first range which removes the majority of the ice and frozen solvent (e.g., at least about 70 wt % (based on the total weight of the tissue form prior to lyopreservation), or at least about 80 wt %, or at least about 90 wt %), followed by (2) a secondary drying step at temperatures within a second range which removes additional ice and frozen solvent from the frozen tissue sample to produce a preserved tissue sample or form having less than about 15 wt % residual moisture (based on the total weight of the preserved tissue form), or having less than about 12 wt %, or less than about 10 wt %, or less than about 8 wt %, or less than about 7 wt %, or less than about 6 wt %, or less than about 5 wt %, or less than about 4 wt %, or less than about 3 wt %, or less than about 2 wt %, or less than about 1 wt % residual moisture. Controlled rate lyopreservation may be desired because it tends to avoid changes in the dried product appearance and characteristics.

“Molarity,” abbreviated as “M,” as used herein describes the molar concentration of a protectant or other solute in a biologically compatible fluid or other solvent in units of moles of the protectant or other solute per liter of solution (total of solute plus solvent). Similarly, molar concentration may be described as “millimolarity,” abbreviated as “mM,” in units of millimoles of the protectant or other solute per liter of solution (total of solute plus solvent).

“Preservation” and “preserving,” as used herein, generally means treating a tissue sample and the endogenous cells therein to minimize or prevent loss, injury, damage, decay or change and maintains or promotes viability of at least a portion of the endogenous cells. Conventionally, “preservation” techniques are understood to include cryopreservation, freezing and lyophilization. However, as used herein “preservation” techniques mean treatment of a tissue sample and the endogenous cells therein, with or without additional components, according to lyopreserving methods performed in the presence of one or more specific protectants or a protectant solution containing them, as described herein.

“Protectants,” as used herein, means substances or compounds which, when contacted with a tissue sample containing a population of viable endogenous cells, promote, facilitate, or enable the preservation of the tissue sample and cells thereof to produce a preserved tissue with at least a portion of the population of cells being viable upon reconstitution. Suitable protectants include, without limitation, one or more substances selected from the group consisting of: sugars, polyphenols (i.e., flavonoids, stillbenes, lignans, phenolic acids), and carotenoids. Suitable sugars include, without limitation, monosaccharides (e.g., glucose, fructose), disaccharides (e.g., trehalose) and polysaccharides (e.g., dextran). Among the suitable flavonoid polyphenols are catechins such as, without limitation, epigallocatechin gallate (EGCG), epicatechin gallate (ECG), epigallocatechin (EGC) and epicatechin (EC). Suitable carotenoids include, without limitation, α-carotene, β-carotene, β-cryptoxanthin, lycopene, lutein, and zeaxanthin. One or more protectants are often, but not necessarily, provided in a protectant solution with a biologically compatible fluid.

“Protectant solution,” as used herein, means a mixture of at least one protectant and a biologically compatible fluid.

“Preserved tissue sample,” and “preserved tissue form,” as used herein, mean a tissue sample or tissue form, respectively, which contained a population of endogenous viable cells prior to preservation and which has been subjected to a preservation technique according to the invention described and contemplated herein, which includes contacting a tissue sample with one or more preservation agents, followed by lyopreserving according to the methods described herein, such that, when reconstituted (i.e., rehydrated by contact with a biologically compatible fluid), at least a portion of the population of endogenous viable cells remains viable after preservation and storage at temperatures above freezing for an extended period of time (e.g., from at least 14 days to 365 days, or at least 1 year, 2 years, 3 years, 5 years, or even more, and including any time between 14 days and 5 years).

“Reconstitute” and “rehydrate” as used in connection with a preserved tissue sample or preserved tissue form comprising same mean the process by which a preserved tissue sample and cells therein are contacted with a biologically compatible fluid, which may reinvigorate dormant cells, e.g., bring cells from a dormant state to a state in which evidence of metabolic activity can be detected. Where a preserved tissue has been frozen, the preserved tissue may be thawed before, during, or after contacting with a biologically compatible fluid. Additionally, a preserved tissue sample or preserved tissue form may be reconstituted or rehydrated in situ, such as after being implanted in a subject, such as by bodily fluids of the subject (e.g., blood, plasma, lymphatic fluid, etc.) which are present at or recruited to the site of implantation, or with biocompatible fluid provided or added to the site of implantation (i.e., internal or external implantation site) by a medical practitioner or other user of the preserved tissue sample or preserved tissue form comprising same.

“Room temperature” as used herein is synonymous with ambient temperature and means from about 15° C. to about 25° C., such as from about 19° C. to about 25° C., or from about 20° C. to about 23° C.

“Sterilization” and “sterilizing” as used herein, in all of their grammatical forms, is any process that renders an object (e.g., a tissue, a container for tissue, or an implement for processing tissue) essentially free from pathogenic organisms and/or viruses by destroying them or otherwise inhibiting their growth or vital activity. Such processes may include exposure of the object to one or more, without limitation, of gamma radiation, electron beam radiation, chemical agents (e.g., alcohol, phenol, ethylene oxide gas, acids, bases, or peroxides), heat, or ultraviolet radiation for sufficient duration and dosages. When sterilization is performed on a finished tissue product in its final packaging, the process may be referred to as “terminal sterilization”.

As used herein, the term “storing,” whether used for tissue samples recovered from a donor (i.e., before or after any processing steps, including contacting with one or more protectants), or preserved tissue samples or preserved tissue forms comprising same (i.e., after lyopreserving), includes any periods of transport or shipping, regardless of temperature.

“Tissue sample” and “biological tissue sample” are used interchangeably herein and mean a piece, slice, slab, section, fragment, chunk, lump, wedge, or any other shape, form or quantity, of tissue which has been recovered, harvested or otherwise derived from one or more donors, living or deceased, human or non-human, which comprises a population of viable endogenous cells and may be fresh, refrigerated (i.e., at temperatures from greater than zero to about 20° C., or frozen (i.e., at temperatures of 0° C. or below). Additionally, the tissue sample may be, include, or be derived from one or more of the following tissue types: adipose, amnion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, placental (including amnion, chorion, amnionchorion, umbilical cord, and Whartons jelly), skin, spleen, stomach, synovial, tendon, testes, umbilical cord, urological, vascular, vein, and a combination thereof. In some embodiments, the tissue is derived from a human or non-human mammal. In some embodiments, the tissue is derived from a human donor. In some embodiments, the human donor is a cadaveric donor.

“Viable,” “viability” and other grammatical forms thereof, as used herein to describe tissue or cells, means having the ability to live, grow, expand or develop, or which are dormant and have potential to live, grow, expand or develop upon reconstitution (e.g., such as by thawing or rehydrating). For example, viability, as applied to cells, can be characterized by having directly or indirectly observable, or measurable, metabolic activity, or which are dormant and have potential to become metabolically active upon reconstitution (e.g., such as by thawing or rehydrating).

Compositions described and contemplated herein are preserved tissue forms comprising preserved tissue samples which are useful as grafts. The preserved tissue forms comprise one or more processed tissue samples which include a population of viable cells, which are endogenous to the tissue samples and at least a portion of which remains viable after the preserved tissue samples or tissue forms comprising them are stored at temperatures above freezing for extended periods of time and then rehydrated. The tissue samples are recovered from one or more donors and then processed with one or more processing steps and techniques which include contacting the tissue samples with one or more protectants, and lyopreserving the resulting tissue-protectant mixture (which may also be described as lyopreserving a tissue sample in the presence of one or more protectants or a protectant solution). The population of viable cells comprise cells endogenous to at least one of the processed tissue samples included in the tissue form. The population of viable cells may be supplemented or combined with exogenous viable cells, which may be autogenic, allogenic, xenogenic, or combinations thereof.

More particularly, prior to lyopreserving (but after other processing steps), the preserved tissue sample comprises a population of endogenous viable cells at least a substantial portion (i.e., at least 98%, or at least 90%, or at least 80%, etc.) of which remain viable after convenient storage of the preserved tissue forms at temperatures above freezing (i.e., greater than 0° C.), for extended periods of time (e.g., up to 14 days, or up to 90 days, or up to 180 days, or up to 365 days, or at least 1 year, 2 years, 3 years, 5 years, or even longer, and including any time between 14 days and 5 years). Such characteristics mean that the tissue forms avoid (1) safety issues sometimes presented by exposure to ultra-low temperatures (i.e., less than about −50° C.) and (2) the inconvenient necessity for thawing the preserved tissue form before use, both of which are encountered when using cryopreserved tissue forms.

The preserved tissue forms described and contemplated herein are useful for tissue repair and reconstruction in recipients having tissue which is damaged, diseased, atrophied or which could otherwise benefit from such treatment (including structural, functional and aesthetic benefits). The preserved tissue forms are useful for tissue repair and reconstruction, at least in part, because the endogenous viable cells retain and/or promote biological activity which facilitates tissue healing, growth and/or generation. Without intending to be limited by theory, it is believed that such biological activity may be provided by the endogenous viable cells themselves and/or growth factors, proteins, or other biologically active substances secreted by, or recruited from surrounding tissue by, the viable cells, or both. This biological activity is in addition to any such activity which may be provided by any growth factors, proteins or other substances already present and remaining in the tissue samples during and after processing.

Without intending to be limited be theory, it is believed that viable cells such as, without limitation, mesenchymal stem cells (MSCs), which are contained in several types of tissue samples (e.g., without limitation, bone, bone marrow, adipose, placenta, etc.) both before and after being subjected to the lyopreserving method described and contemplated herein, are known to secrete several types of biologically active substances including growth factors, cytokines, chemokines, and other proteins which cause or contribute to paracrine effects (immunomodulatory, anti-apoptotic, supportive (e.g., stimulation of mitosis, proliferation and differentiation), angiogenic, anti-scaring, chemoattractant, etc.) known to enhance wound healing and tissue reconstruction. Specific examples of such paracrine signaling substances secreted by one or more types of viable cells, include, without limitation, vascular endothelial growth factor (VEGF), hepatocyte growth factor (HGF), insulin-like growth factor-1 (IGF-1), placental growth factor (plGF), transforming growth factor beta (TGF-β), basic fibroblast growth factor (bFGF), granulocyte-macrophage colony-stimulating factor (GM-CSF), prostaglandin E2 (PGE-2), indoleamine 2,3-dioxygenase (IDO), interleukin (IL)-6, interleukin (IL)-12, IL-1 receptor antagonist (IL-Ira), tumor necrosis factor alpha (TNF-α), monocyte chemoattractant protein-1 (MCP-1/also known as CCL2), macrophage inflammatory proteins (MIP-1α/CCL3, MIP-1β/CCL4, MIP-3α/CCL20, eotaxin-3/CCL26, etc.), and many others.

In accordance with the preserved tissue forms and methods for making and using them which are described and contemplated herein, after being subjected to the lyopreserving methods described and contemplated herein, preserved tissue forms contain both paracrine signaling substances (growth factors, cytokines, chemokines, etc.) already secreted by endogenous viable cells of the tissue sample (before and during lyopreserving), as well as viable cells which continue to secrete such paracrine signaling substances after lyopreserving and rehydration. Accordingly, preserved tissue samples and preserved tissue forms comprising them which are produced in accordance with the lyopreserving methods described and contemplated herein, upon rehydration, behave and function as conditioned media containing biologically active substances which cause or contribute to paracrine effects and other beneficial biological activities which cause, facilitate or enhance wound healing and tissue reconstruction when implanted in a host, patient or subject. Furthermore, upon rehydration, such preserved tissue samples and preserved tissue forms comprising them also produce and provide additional quantities of biologically active substances which continue to provide such beneficial healing and reconstructive activity at the site of implantation. As will be understood by persons of ordinary skill in the relevant art, the site of implantation may be within or on an external surface of a subject, or a combination or both.

The recovered tissue samples may be, include, or be derived from one or more of the following tissue types: adipose, amnion, artery, bone, cartilage, chorion, colon, dental, dermal, duodenal, endothelial, epithelial, fascial, gastrointestinal, growth plate, intervertebral disc, intestinal mucosa, intestinal serosa, ligament, liver, lung, mammary, meniscal, muscle, nerve, ovarian, parenchymal organ, pericardial, periosteal, peritoneal, placental (including amnion, chorion, amnionchorion, umbilical cord, and Wharton's jelly), skin, spleen, stomach, synovial, tendon, testes, umbilical cord, urological, vascular, vein, and a combination thereof. Preferred tissue types include, without limitation: adipose, fascia, dermis, bone, cartilage, muscle, placenta (including amnion, chorion, amniochorion, Wharton's jelly, and umbilical cord), placental disk, and combinations thereof.

As described below, embodiments of the present invention include preserved tissue forms comprising at least one processed tissue sample which has been contacted with one or more protectants and lyopreserved (i.e., at least one preserved tissue sample), methods for producing such preserved tissue forms, and methods for using such preserved tissue forms. Although the various embodiments are described in further detail in connection with tissue samples comprising bone (e.g., cancellous bone), cartilage, and placental (e.g., amnion and chorion) tissues, it should be clear and understood by persons of ordinary skill in the relevant art that the present invention contemplates and includes embodiments comprising other tissue types, such as those listed above. It should also be clear and understood that the present invention contemplates and includes embodiments comprising more than one tissue type and that those tissue types may be processed the same way or differently. Additionally, the present invention contemplates and includes embodiments in which the preserved tissue form further comprises materials, substances or components, which may be natural or synthetic, in addition to one or more processed tissue samples at least one of which has been contacted with one or more protectants and lyopreserved. All such embodiments, and other reasonably overlapping or derived from those described herein are within the scope of the present invention.

The one or more preserved tissue samples included in any particular preserved tissue form may be autografts (i.e., recovered from the same individual donor as the intended recipient), allografts (i.e., recovered from a different individual donor of the same species as the intended recipient), xenografts (i.e., recovered from an individual donor of a different species as the intended recipient), or combinations thereof.

As will be familiar to persons of ordinary skill in the relevant art, the tissue samples are processed by applying steps and techniques which are selected depending on the type of tissue recovered, the desired characteristics of the processed tissue samples, the intended tissue form and its intended use (i.e., the particular intended recipient, the tissue type to be treated, the condition to be treated and the desired results of said treatment). Accordingly, the method for producing a preserved tissue form comprises processing a tissue sample by performing one or more of the following steps:
(A) recovering a tissue sample from a donor according to accepted ethical and sterile procedures;
(B) optionally, cleaning the tissue sample;
(C) optionally, disinfecting the tissue sample;
(D) optionally, modifying one or more of the size, shape and other physical characteristics of the tissue sample by applying one or more physical treatments, chemical treatments, or combinations thereof;
(E) contacting the tissue sample with one or more protectants, which may be in solution with a biologically compatible fluid, for a period of time, to form a tissue-protectant mixture comprising a quantity of tissue sample and one or more protectants;
(F) optionally, prior to lyopreserving, storing the tissue-protectant mixture, for a period of storage time, at a storage temperature (e.g., less than −80° C., or less than −50° C.), optionally in contact with storage media, preservatives, priming media, or combinations thereof;
(G) optionally, prior to lyopreserving, incubating the tissue-protectant mixture at an incubation temperature, for a period of incubation time, optionally in contact with storage media, preservatives, priming media, or combinations thereof;
(H) lyopreserving the tissue-protectant mixture by first freezing the tissue-protectant mixture, and then drying the frozen tissue-protectant mixture (optionally under vacuum) to produce a processed (lyopreserved) tissue sample.

The resulting processed tissue sample is a lyopreserved tissue sample which may, by itself, be suitably useful as a preserved tissue form and implanted into a subject. Alternatively, the method for producing a preserved tissue form may further comprise combining the processed preserved tissue sample with one or more additional components, either prior to, during or after the lyopreserving step (H).

After recovery or harvest of the tissue sample from one or more donors, the tissue sample includes a fresh population of viable cells which are endogenous to the tissue sample. Before performing the step of contacting (E) the tissue sample with one or more protectants, but after all other desired processing steps are performed, the processed tissue sample includes a pre-contact (or Baseline) population of viable cells which is at least a portion of the fresh population of viable cells. After performing all desired processing steps and the step of contacting (E) the tissue sample with one or more protectants, but prior to performing the step of lyopreserving (H), the processed tissue form (with or without one or more additional components) includes a post-contact (or pre-lyopreservation/“pre-lyo”) population of viable cells which is at least a portion of the pre-contact population of viable cells.

After lyopreserving (H), the resulting preserved tissue form (with or without one or more additional components, in addition to the preserved tissue sample) includes a preserved TO (or “Week 0,” “post-lyopreservation,” or “post-lyo”) population of viable cells which is at least a portion of the post-contact population of viable cells. The T0 (“Week 0,” “post-lyopreservation,” or “post-lyo”) population of viable cells should be measured shortly after the lyopreserving step (H) is completed, such as within about 7 days, or within about 96 hours, or within about 48 hours, or even within about 24 hours, of completing the lyopreserving step (H). The preserved tissue form may be stored at temperatures above freezing, for an extended period of time, after which the preserved tissue form includes a retained population of viable cells, which is at least a portion of the T0 population of viable cells. The fresh, pre-contact (“Baseline”), post-contact (pre-lyopreservation, or pre-lyo), T0 (“Week 0,” “post-lyopreservation,” or “post-lyo”), and retained populations of viable cells each comprise viable cells endogenous to at least one tissue sample which has been subjected to processing as described and contemplated herein to produce the preserved tissue form. The viable endogenous cells of a preserved tissue sample are those which were present in the original unprocessed tissue sample and have not been removed from that tissue sample during processing and lyopreserving. In some embodiments, one or more of the fresh, pre-contact (“Baseline”), post-contact (pre-lyopreservation, or pre-lyo), T0 (“Week 0,” “post-lyopreservation,” or “post-lyo”), and retained populations of viable cells may each be supplemented or combined with exogenous viable cells, which may be, independently of one another, autogeneic, allogeneic, xenogeneic, or combinations thereof, and may have been added to the tissue form at any point during the processing and preservation steps. In some embodiments, the retained population of viable cells is more than 50% of the T0 population of viable cells, such as more than 60%, or more than 70%, preferably more than 80% or 85%, and most preferably more than 90%, or 95%, or even 98%.

Storage of the preserved tissue form at temperatures above freezing includes, for example without limitation, storage at room temperatures (i.e., from about 19° C. to about 25° C.), or refrigeration temperatures (i.e., from greater than 0° C. to about 10° C.), or intermediate temperatures (i.e., from greater than 10° C. to less than about 19° C.). Extended periods of time for which preserved tissue forms may be stored and still have a retained population of viable cells includes, for example without limitation, from at least 14 days to 365 days or more, such as at least 14 days, or at least 28 days, or at least 56 days, or at least 70 days, or at least 90 days, or at least 180 days, or at least 270 days, or at least 365 days, or at least 1 year, 2 years, 3 years, or even longer, and including any time between 14 days and 5 years. It is noted that periods of storage may include or overlap with one or more periods of time during which the preserved tissue sample or preserved tissue form comprising same is transported or shipped from one location to another, and during at least a portion of which the temperature to which the preserved tissue sample or preserved tissue form comprising same is exposed may be at or below freezing, or above freezing.

The preserved tissue form provides several benefits and advantages, including but not limited to: (1) providing a preserved tissue form containing a (retained) population of viable cells which may be conveniently stored at temperatures above freezing until use, (2) resolving customer safety concerns presented by exposure to ultra-low temperatures (e.g., below −50° C. or even −80° C.) relative to conventional cryopreserved tissue forms, and (3) eliminates the need for thawing time prior to use as compared to conventional cryopreserved and frozen tissue forms. For tissue processors and tissue form producers, the ability to store the tissue forms above freezing temperature eliminates the need for costly storage resources (dry ice, liquid nitrogen, tanks and freezers, etc.) and removes certain limitations on tissue shipment (limit on shipment time and size due to dry ice).

Each of the above-listed steps (A)-(H) of the method for producing a preserved tissue form comprising processing a tissue sample will now be described and explained in further detail.

Tissue samples are recovered from a donor (A) according to accepted ethical and sterile procedures, which involve donor screening, obtaining donor releases and informed consent, tissue screening and testing and tissue transport to facilities for further processing. The tissue samples may be stored, such as in a biologically compatible fluid or not, for a period of time after recovery from the donor(s) (A) and prior to shipping/transport, or during shipping/transport, or even for a period of time after arriving at a processing facility but prior to commencement of processing steps (e.g., cleaning (B), disinfecting (C), modifying the size, shape, or other physical characteristics (D), contacting with one or more protectants (E), storing (F), incubating (G), lyopreserving (H), etc.). In some embodiments, recovered tissue samples may be held or stored in another kind of solution, such as, without limitation, a red cell lysis buffer such as ACK (Ammonium-Chloride-Potassium) Lysing Buffer which is intended to facilitate removing unwanted blood from the samples.

In various embodiments, the step of cleaning (B) a recovered tissue sample is typically intended to isolate the desired tissue type by removing unwanted tissue and other materials and may, for example, include one or more of the following processes: (1) debriding or otherwise separating the recovered tissue sample to remove and separate unwanted tissue from desired recovered tissue; and (2) removing unwanted materials and substances including, without limitation, blood, lipids, debris, and unwanted tissue by rinsing or washing the recovered tissue sample, for a period of cleaning time, at least once, with one or more fluids such as, without limitation, buffered or unbuffered saline solution, lactated Ringer's solution, balanced salt solution, basal medium, water, or combinations thereof.

A suitable period of cleaning time for rinsing or washing during the step of cleaning (B) the tissue sample is typically, for example without limitation, at least 2 seconds, or at least 10 minutes, or at least 20 minutes, or from about 2 seconds to about 2 hours, such as from about 2 seconds to about 5 minutes, or from about 5 minutes to about 20 minutes, or from about 20 minutes to about 2 hours. The step of (B)(2) removing unwanted materials and substances from a recovered tissue sample may also, or alternatively, be performed by contacting the recovered tissue sample with supercritical carbon dioxide for a period of time of from about 2 minutes to about 60 minutes, such as from about 5 minutes to about 60 minutes, or from about 5 minutes to about 40 minutes, or from about 5 minutes to about 30 minutes, or from about 5 minutes to about 20 minutes.

In various embodiments, the step of disinfecting (C) a recovered tissue sample typically, but without limitation, includes contacting, rinsing, or soaking the tissue sample, for a period of disinfecting time, with one or more disinfecting agents or a disinfecting solution comprising one or more disinfecting agents. As will be recognized by persons of ordinary skill in the relevant art, a suitable period of disinfecting time for the step of disinfecting (C) will depend on the type and concentration of disinfecting agent or disinfecting solution and is typically, for example without limitation, at least 0.5 seconds, or from about 0.5 seconds to about 2 hours, such as from about 0.5 seconds to about 5 minutes, or from about 5 minutes to about 20 minutes, or from about 20 minutes to about 2 hours, or from about 30 minutes to about 2 hours, or from about 30 minutes to about 1.5 hours, or from about 30 minutes to about 1 hour. Suitable disinfecting agents include, without limitation, chlorine and chlorine compounds (e.g., hydrochloric acid), alcohols (e.g., ethanol, isopropyl alcohol), peracetic acid, surfactants (e.g., Triton X-100, Tween, Sodium Dodecyl Sulfate), antibiotics (e.g., amoxicillin, penicillin, gentamicin, amphotericin, doxycycline, azithromycin, vancomycin, etc.), antimycotics (e.g., amphotericin B, nystatin), combinations thereof, and solutions containing same. In some embodiments, for example, the step of disinfecting (C) comprises soaking the tissue sample in a sufficient volume of peracetic acid solution for a sufficient amount of disinfecting time so as to produce a disinfected tissue sample, wherein the disinfected tissue sample has a sterility assurance level of at least 10-6. The sterility of the disinfected tissue sample is determined by the method described in International Standards ISO 14937:2009. Furthermore, a sufficient volume of peracetic acid would provide, for example without limitation, a ratio of the tissue sample surface area (cm²) to the volume of peracetic acid solution (ml) of from about 0.01 cm²/ml to about 0.65 cm²/ml.

The step (D) of modifying size or shape of the tissue sample includes, without limitation, increases or decreases in one or more dimensions (e.g., without limitation, changes to one of more of diameter, width, length, height, thickness, etc.) and formation or molding recovered tissue into any desired shape, whether by manual manipulation or with the use of a container or mold. Possible shapes for the tissue samples, processed tissue samples, and preserved tissue forms include, without limitation, particles, strips, chunks, pieces, blocks, sheets, slivers, ribbons, branched and unbranched elongated elements, filaments, fibers, three dimensional geometric shapes such as symmetric and asymmetric spheres, regular and irregular polyhedrons, cones, pyramids, other three dimensional forms having one or more planar or curved surfaces, and irregular three dimensional forms. As will be understood and practicable by persons of ordinary skill in the relevant art, processed tissue samples and preserved tissue forms comprising them may be produced in the form (i.e., have the shape) of, for example without limitation, one or more of particulates, fibers, chunks or pieces, etc. (i.e., a first shape) and then be molded (reshaped) into another desired shape, for example without limitation, sheets, blocks, cylinders, plugs or other three-dimensional shape (i.e., a second shape), such as by manual manipulation, manipulation using a device, or using a mold or other container to impart a desired shape, with or without agitation, drying, etc. Modifications to other physical characteristics include, without limitation, removal of lipids such as for adipose tissue, demineralizing such as for bone tissue, changes to the molecular structure of collagens or proteins in a tissue sample (e.g., digestion, hydrolysis, cleavage, crosslinking, etc.), and increasing or decreasing flexibility, density, compressibility, elasticity, etc.

In some embodiments, the optional step (D) of modifying size, shape or other physical characteristics of the tissue sample includes, for example without limitation, one or more of the following physical processes: cutting, slicing, cleaving, chopping, grating, grinding, milling, fragmenting, blending, homogenizing, extruding, fracturing, separating, pressing, molding, manual manipulation, heating, cooling, freezing, and the like, using devices known now or in the future to persons of ordinary skill in the relevant art. Selection of a physical process and a device for performing the selected physical process is within the ability of persons of ordinary skill in the relevant art based on this disclosure and the knowledge generally possessed by and available to such persons and will depend on various factors including, without limitation, the type of tissue sample being treated, the condition intended to be treated by the resulting tissue form comprising the processed tissue sample, and the desired result of such treatment.

In some embodiments, the step (D) of modifying size, shape or other physical characteristics of the recovered tissue sample includes, for example without limitation, one or more of the following chemical processes: digesting, cleaving, dissolving, disintegrate, dissociating, hydrolyzing, fragmenting, separating, extracting, absorbing, desorbing, aggregating, linking, and the like, using agents and techniques known now or in the future to persons of ordinary skill in the relevant art. Suitable agents and techniques include, for example without limitation, contacting the tissue sample with an enzyme (e.g., a kinase, an amylase, telomerase, trypsin, collagenase, pepsin, lipase, etc.), an acid, a base, an ionic substance, a surfactant, and the like, and combinations thereof, with or without heating or cooling for a period of time sufficient to accomplish the desired degree of chemical alteration of the tissue sample. Selection of a chemical process and an agent and/or technique, including the quantity of the agent and length of time for contacting the tissue sample with the agent are within the ability of persons of ordinary skill in the relevant art based on this disclosure and the knowledge generally possessed by and available to such persons and will depend on various factors including, without limitation, the type of tissue sample being treated, the condition intended to be treated by the resulting tissue form comprising the processed tissue sample, and the desired result of such treatment.

Generally, any combination of one or more physical and chemical processes may be performed, in any order, to accomplish the step (D) of modifying size, shape or other physical characteristics of the tissue sample. In some embodiments, more than one physical process may be performed. In some embodiments, more than one chemical process may be performed. In some embodiments, the step (D) of modifying size, shape or other physical characteristics of the tissue sample may include at least one physical process and at least one chemical process. In some embodiments, one or more chemical processes may be performed prior to any physical processes, and in other embodiments, one or more physical processes may be performed prior to any chemical processes. In some embodiments, one or more chemical processes may be performed both prior to and after one or more physical processes. In some embodiments, one or more physical processes may be performed both prior to and after one or more chemical processes. Additionally, the physical and chemical processes of step (D) may be performed in any order as desired.

As will be understood by persons of ordinary skill in the relevant art, in addition to being optional depending on the type and source of the tissue sample, the steps of cleaning (B), disinfecting (C), and modifying one or more of the size, shape and other physical characteristics of (D) the tissue sample may each be performed with one or more repetitions and in any order, at the discretion of persons of ordinary skill. For example, without limitation, these steps may be performed sequentially as listed above. In some embodiments, without limitation, a recovered tissue sample may first be disinfected (C) and then cleaned (B), with or without subsequent modifying one or more physical characteristics (D). In other embodiments, a recovered tissue sample may be cleaned (B), disinfected (C), then cleaned again (B), prior to, optionally, modifying one or more physical characteristics (D). In other embodiments, for example, without limitation, a recovered tissue sample may be cleaned (B), then cleaned (B) again (such as with different cleaning agents or by different cleaning techniques, etc.), followed by disinfecting (C), with or without subsequent modifying one or more physical characteristics (D). In still other embodiments, without limitation, a tissue sample may be subjected to a step of modifying one or more of its physical characteristics (D), followed by one or more cleaning steps (B), then further modifying one or more of physical characteristics (D) of the tissue sample, followed by disinfecting (C), and then an additional cleaning step (B). Any such number and order of the optional steps of cleaning (B), disinfecting (C), and modifying one or more physical characteristics (D), as well as other arrangements of such steps, are possible and suitable, as is within the ability of persons of ordinary skill in the relevant art to determine, depending on the source, type and intended use of the recovered tissue.

Furthermore, the tissue sample is contacted, in step (E) listed above, with one or more protectants (in solution or not) for a period of contacting time to form a tissue-protectant mixture comprising a quantity of tissue sample and the one or more protectants. The period of contacting time is from greater than zero seconds and extends until lyopreserving is commenced, such as up to about 48 hours. For example without limitation, the period of contacting time for the step of (E) contacting the tissue sample with one or more protectants may be at least about 10 minutes, or at least about 15 minutes, or at least about 20 minutes, or at least about 60 minutes, or at least about 120 minutes, or at least about 180 seconds, or at least 240 minutes, or at least 6 hours, or at least 10 hours, or any value from greater than zero to about 48 hours. It is noted that in some embodiments, the tissue sample or portions thereof may be contacted with (e.g., rinsed, soaked, stored in, etc.) one or more protectants (in solution or not) at any time during processing, before during or after any of the steps of the method for producing the preserved tissue forms described herein. Any periods of time for which the tissue sample or portions thereof is contacted with one or more protectants before or during any of the steps of (A) recovering, (B) cleaning, (C) disinfecting, (D) modifying, (E) storing or (F) incubating, the tissue sample are not necessarily intended to be included in the period of contacting time which occurs during the contacting step (E) performed prior to lyopreserving as discussed above. In other words, in some embodiments, the tissue sample or a portion thereof may be contacted with one or more protectants (in solution or not) for a period of time longer than about 48 hours, particularly, when the contact also occurs before or concurrently with one or more of the steps performed prior to the step of (H) lyopreserving.

In some embodiments, such as those in which the tissue sample is a bone tissue sample, the period of contacting time may, without limitation, be at least about 20 minutes, or at least about 30 minutes, or at least about 60 minutes, or at least about 120 minutes, or at least about 180 minutes, or at least 240 minutes, or any value between greater than zero and up to about 6 hours, or up to about 7 hours, or up to about 8 hours, or even up to about 9 hours. In some embodiments, such as those in which the tissue sample is a cartilage tissue sample, the period of contacting time may, without limitation, be at least about 10 minutes, or at least about 30 minutes, or at least about 60 minutes, or at least about 120 minutes, or at least about 180 minutes, or at least 240 minutes, or at least 300 minutes, or any value between greater than zero and up to about 6 hours, or up to about 7 hours, or up to about 8 hours, or even up to about 9 hours. In some embodiments, such as those in which the tissue sample is a placental tissue sample such as, without limitation, one or a combination of amnion and chorion tissues, the period of contacting time may, without limitation, be at least about 15 minutes, or at least about 30 minutes, or at least about 60 minutes, or at least about 120 minutes, or at least about 180 minutes, or at least 200 minutes, or any value between greater than zero and up to about 4 hours, or up to about 5 hours, or up to about 6 hours, or up to about 7 hours, or up to about 8 hours, or even up to about 9 hours.

Examples of protectants suitable for use with the method described herein include, without limitation, one or more of: low molecular weight non-reducing sugars (e.g., trehalose, sucrose, glucose, etc.), dextran, catechins (e.g., epigallocatechin (EGG), epigallocatechin gallate (EGCG), etc.), carotenoids (α-carotene, β-carotene, β-cryptoxanthin, lycopene, lutein, zeaxanthin, and other naturally occurring carotenoids), glycerol, antioxidants (e.g., ascorbic acid (vitamin C), vitamin E, etc.), and late embryogenesis abundant (LEA) proteins. Preferred protectants include trehalose, EGCG, and combinations thereof. EGCG is also known to have antioxidant properties and, therefore, is a highly suitable protectant.

Where more than one protectant is employed, the protectants may be contacted with the tissue sample in any order and combination. For example, without limitation, in some embodiments, a first protectant may be contacted with the tissue sample, and then an additional one or more (i.e., second, third, fourth, etc.) protectants may be added sequentially, or in combinations. In some embodiments, two or more protectants may be contacted with the tissue sample concurrently, either by separate but concurrent addition to the tissue sample, or by mixing the protectants together and then adding the mixed protectants to the tissue sample. Similarly, in some embodiments, two or more protectants may be combined with one another to form a first protectant mixture, and two or more different protectants may be combined with one another to form a first protectant mixture, and then the first and second protectant mixtures added to the tissue sample, separately but concurrently, or sequentially, or by first mixing the first and second protectants mixtures together and then adding the resulting combined mixtures to the tissue sample.

The one or more protectants will typically, but do not have to, be in solution with a biologically compatible fluid. Suitable biologically compatible fluids include, for example without limitation, one or more of: saline, PBS, Hank's balanced salt solution (HBSS), Hyclone® growth media containing fetal bovine serum (commercially available from Thermo Fisher Scientific of Carlsbad, California, U.S.A.), Dulbecco's Modified Eagle's medium (DMEM), Basal Medium Eagle (BME), and derivatives and combinations thereof. In some embodiments, after all protectants, fluids and any additional ingredients have been combined to form the protectant solution, the solution may be sterilized, such as by exposure to ultraviolet or gamma radiation, filtering through a filter, such as a 0.2 um filter, or other techniques known to persons of ordinary skill in the art and discussed further hereinbelow.

In an exemplary embodiment, the protectant solution comprises trehalose and EGCG dissolved in PBS. Where trehalose is at least one of the protectants in the protectant solution, trehalose may, for example, be present in a concentration of from about 0.1 M to about 1 M, such as from about 0.2 M to about 0.6 M, or from about 0.3 M to about 0.5 M, or about 0.4 M, or about 0.5 M. Where EGCG is at least one of the protectants in the protectant solution, EGCG may, for example, be present in a concentration of from about 0.5 mM to about 8 mM, such as from about 1 mM to about 6 mM, or from about 3 mM to about 5 mM, or about 4 mM.

In some embodiments, additional ingredients may be combined with the protectant(s) or the protectant solution. Suitable additional ingredients for the protectant solution include, without limitation, one or more of: nonionic surfactants (e.g., Polysorbate 80), glucose, mannitol, amino acids (e.g., glycine), salts, vitamins, human serum albumin (HSA), bovine serum albumin (BSA).

In some embodiments, for example, an exemplary protectant solution comprises trehalose and EGCG dissolved in either Hyclone® media or phosphate buffered solution (PBS) at a concentration of from about 0.1 to about 0.5 M and from about 1.0 to about 4.0 mM, respectively. In one embodiment, for example, the protectant solution may comprise 1×PBS, 0.4 M trehalose and 4.0 mM EGCG. In some embodiments, the aforesaid exemplary protectant solutions further comprise dextran, glycerol, or a combination of both.

In some embodiments, the step (E) of contacting the tissue sample with one or more protectants may be performed at room temperature (i.e., from about 19° C. to about 25° C.) and ambient pressure (typically from about 0.8 atmosphere (atm) to about 1.05 atm, which is from about 85 kilopascals (kPa) to about 107 kPa, depending on altitude). In some embodiments, the tissue sample is contacted with one or more protectants and subjected to heating (e.g., application of or exposure to one or more temperatures above about 25° C.) or cooling (e.g., application of or exposure to one or more temperatures below about 19° C.), for all or a portion of the contacting time.

After performing the step of (E) contacting the tissue sample with one or more protectants, or protectant solution comprising same, the resulting tissue-protectant mixture is subjected to the step of (H) lyopreserving. In some embodiments, after (E) contacting with protectants or protectant solution, but before (H) lyopreserving, the tissue-protectant mixture may be subjected to one or more other intermediate treatment (i.e., processing) steps, such as one or more of cleaning (B), disinfecting (C), and modifying one or more of the size, shape and other physical characteristics (D), at the discretion of persons of ordinary skill. For example, without limitation, in some embodiments, the tissue-protectant mixture may be soaked or rinsed with a biocompatible fluid (e.g., PBS, a culture media, a buffered isotonic solution, etc.), or even with a second protectant solution (either of the same composition already used, or different composition), prior to performing a lyopreserving step (H).

The step (H) of lyopreserving the tissue-protectant mixture is typically performed by first freezing the tissue-protectant mixture, and then drying the frozen tissue-protectant mixture under vacuum to produce a processed tissue sample which has a retained population of viable cells after rehydration.

In some embodiments, the freezing phase of the lyopreserving step (H) may, for example without limitation, be performed at one or more temperatures in a range of from about −80° C. to about −4° C., such as from about −70° C. to about −4° C., or from about −50° C. to about −4° C. In some embodiments, the freezing phase of the lyopreserving step (H) is performed at a rate of from about 0.1° C./minute to about 10° C./minute, such as from about 0.1° C./minute to about 5° C./minute, or from about 0.1° C./minute to about 2° C./minute. The freezing phase of the lyopreserving step (H) may be performed over a total freezing time of from about 5 minutes to about 300 minutes, or any range therebetween, such as without limitation from about 5 minutes to about 200 minutes, or from about 5 minutes to about 250 minutes, or from about 5 minutes to about 180 minutes, or from about 5 minutes to about 130 minutes, or from about 5 minutes to about 100 minutes, or from about 5 minutes to about 90 minutes, or from about 5 minutes to about 75 minutes, or from about 5 minutes to about 60 minutes, or from about 5 minutes to about 30 minutes, or from about 10 minutes to about 300 minutes, or from about 15 minutes to about 200 minutes, or from about 20 minutes to about 300 minutes, or from about 30 minutes to about 300 minutes, or from about 30 minutes to about 300 minutes, or from about 45 minutes to about 300 minutes, or from about 60 minutes to about 300 minutes, or from about 75 minutes to about 300 minutes, or from about 90 minutes to about 300 minutes, or from about 90 minutes to about 180 minutes, or from about 90 minutes to about 135 minutes.

The drying phase of the lyopreserving step (H) removes water and other solvents from the frozen tissue sample and is performed for a period of total drying time. In some embodiments, the drying phase of the lyopreserving step (H) is performed under vacuum in a single step, either at a constant temperature or at varied temperatures (e.g., two or more steps) within a single range. In some embodiments, the drying phase of the lyopreserving step (H) is performed, under vacuum or not, in two steps including a primary drying step at one or more temperatures within a first range which removes the majority of the ice and frozen solvent (e.g., at least about 70 wt % of the frozen water is removed), followed by (2) a secondary drying step at one or more temperatures within a second range which removes additional ice and frozen solvent from the frozen tissue sample to produce a preserved tissue sample having less than about 10 wt % water. In some embodiments, the drying phase of the lyopreserving step (H) is performed in three or more steps, analogous to those described above. Additionally, it is noted that the ranges of temperatures employed during the one or more steps of the drying phase (e.g., first range, second range, etc.) may overlap or not. Regardless of the number of drying steps or temperature ranges employed to perform the drying phase of the lyopreserving step (H), controlled rate lyopreserving may be desired because it tends to avoid changes in the dried product appearance and characteristics.

By application of a vacuum during the drying phase of lyopreserving the tissue sample, the drying phase may, for example without limitation, be performed at a pressure of from about 0.013 kPa to about 0.13 kPa (i.e., from about 100 milliTorr (mTorr) to about 1000 mTorr), for example from about 0.013 kPa to about 0.1 kPa (i.e., from about 100 mTorr to about 750 mTorr), or from about 0.013 kPa to about 0.066 kPa (i.e., from about 100 mTorr to about 500 mTorr). The vacuum and pressure during the drying phase of the lyopreserving step (H) need not remain constant throughout the drying phase. In some embodiments, the vacuum and pressure during the drying phase varies, by gradual change rates or by step changes, in a range of from about 0.013 kPa to about 0.13 kPa (i.e., from about 100 mTorr to about 1000 mTorr), with possible exemplary sub-ranges as stated above.

The total drying time for which the drying phase of the lyopreserving step (H) is performed may, for example without limitation, be up to about 48 hours, or from about 2 hours to about 48 hours, or any range therebetween, such as without limitation from about 2 hours to about 45 hours, or from about 4 hours to about 40 hours, or from about 6 hours to about 36 hours, or from about 10 hours to about 40 hours, or from about 10 hours to about 36 hours, or from about 10 hours to about 30 hours, or from about 12 hours to about 40 hours, or from about 12 hours to about 36 hours, or from about 12 hours to about 30 hours, or from about 12 hours to about 24 hours, or from about 18 hours to about 40 hours, or from about 18 hours to about 36 hours, or from about 18 hours to about 24 hours.

In some embodiments, wherein the drying phase of the lyopreserving step (H) is performed under vacuum in a single step at a constant temperature, the constant temperature may be in a range of from about −50° C. to about 25° C., for example without limitation, from about −50° C. to about −15° C., or from about from about −50° C. to about −10° C., or from about from about −35° C. to about −10° C., or from about from about −25° C. to about −10° C., or from about from about −20° C. to about −10° C., or from about from about −20° C. to about 0° C., or from about from about −15° C. to about 25° C., or from about from about −10° C. to about 25° C., or from about from about −10° C. to about 20° C., or from about from about −10° C. to about 10° C., or from about from about −10° C. to about 20° C., or from about from about 0° C. to about 20° C., or from about from about 0° C. to about 25° C. In some embodiments, wherein the drying phase of the lyopreserving step (H) is performed under vacuum varied temperatures (e.g., two or more steps), the varied temperatures may be within a single range of from about −50° C. to about 25° C., for example without limitation, from about-50° C. to about −15° C., or from about from about −15° C. to about 25° C., or from about from about 0° C. to about 25° C. Whether a constant temperature or varied temperatures in a single range are used, the resulting preserved tissue form, which comprises a processed tissue sample, with or without additional components, has a water content of less than about 10 wt %, or less than about 8 wt %, or less than about 7 wt %, or less than about 6 wt %, or less than about 5 wt %, or less than about 4 wt %, or less than about 3 wt %, or less than about 2 wt %, or less than about 1 wt %, based on the total weight of the preserved tissue form.

In some embodiments, the drying phase of the lyopreserving step (H) is performed under vacuum in two steps which include a primary drying step at one or more temperatures within a first range which removes the majority of the ice and frozen solvent (e.g., at least about 70 wt %, or at least about 80 wt %, or at least about 90 wt %), followed by (2) a secondary drying step at one or more temperatures within a second range which removes additional ice and frozen solvent from the frozen tissue sample to produce a preserved tissue form, which comprises a processed tissue sample, with or without additional components, having less than about 10 wt %, or less than about 8 wt %, or less than about 7 wt %, or less than about 6 wt %, or less than about 5 wt %, or less than about 4 wt %, or less than about 3 wt %, or less than about 2 wt %, or less than about 1 wt % water.

As mentioned above, the first and second ranges for drying temperatures may overlap or not. In some embodiments, for example without limitation, the first range of drying temperatures is from about −100° C. to about 15° C., or any range therebetween, such as without limitation from about −80° C. to about 0° C., or from about from about −60° C. to about 0° C., or from about from about −50° C. to about 0° C., or from about from about −50° C. to about −15° C., or from about from about −50° C. to about −10° C., or from about −45° C. to about 0° C., or from about −45° C. to about −5° C., or from about −45° C. to about −15° C., or from about −60° C. to about 15° C., or from about −45° C. to about 5° C., or from about from about −30° C. to about 15° C., or from about from about −20° C. to about 15° C., or from about from about −15° C. to about 15° C., or from about from about −10° C. to about 15° C., or from about from about −10° C. to about 15° C., or from about from about −10° C. to about 10° C.

The time for which a primary drying step is performed may be, for example without limitation, up to about 40 hours, or from about 1 hour to about 40 hours, or any range there between, such as without limitation, or from about 2 hours to about 36 hours, or from about 6 hours to about 36 hours, or from about 12 hours to about 30 hours, or from about 12 hours to about 24 hours.

Generally, the secondary drying step is performed at one or more temperatures higher than drying temperatures employed during the primary drying step. In some embodiments, the secondary drying step is performed at one or more temperatures up to about 45° C., such as up to about 40° C., or up to about 35° C. In some embodiments, for example without limitation, the second range of drying temperatures is from about −25° C. to about 45° C., or any range therebetween, such as without limitation from about −25° C. to about 45° C., or from about −20° C. to about 40° C., or from about −25° C. to about 35° C., or from about −15° C. to about 45° C., or from about −15° C. to about 35° C., or from about −15° C. to about 25° C., or from about −10° C. to about 45° C., or from about −10° C. to about 35° C., or from about −10° C. to about 25° C., or from about −5° C. to about 35° C., or from about −5° C. to about 25° C., or from about 0° C. to about 35° C., or from about 0° C. to about 25° C.

The time for which a secondary drying step is performed may be, for example without limitation, up to about 40 hours, or from about 1 hour to about 40 hours, or any range therebetween, such as without limitation from about 2 hours to about 36 hours, or from about 6 hours to about 36 hours, or from about 6 hours to about 30 hours, or from about 6 hours to about 20 hours, or from about 6 hours to about 16 hours.

For example, in some embodiments, where the first range of drying temperatures is from about −50° C. to about −15° C., the second range of drying temperatures may be from about −15° C. to about 25° C., such as from about −10° C. to about 25° C. In some embodiments, where the second range of drying temperatures is from about −50° C. to about −10° C., the second range of drying temperatures may be from about −15° C. to about 25° C., such as from about −10° C. to about 25° C.

When lyopreserving is performed in accordance with the method described above, in the presence of one or more protectants described above, or a protectant solution containing them, the tissue form produced thereby comprises a processed tissue sample, with or without additional components, and contains a retained population of viable cells even after storage at temperatures above freezing for extended periods of time. Moreover, the retained population of viable cells is a substantial portion of the T0 population of viable cells that was present in the tissue sample immediately after lyopreserving (H) and prior to storage at room or ambient temperatures for a period of time (e.g., from at least 14 days to 365 days, or at least 1 year, 2 years, 3 years, 5 years, or even more, and including any time between 14 days and 5 years). In some embodiments, the retained population of viable cells in the preserved tissue form is measurably viable after rehydration of the tissue form. In some embodiments, at least a portion of the retained population of viable cells in the preserved tissue form are dormant and become measurably viable after reconstitution with a biologically compatible fluid. In some embodiments, the retained population of viable cells is more than 50% of the T0 population of viable cells, such as more than 60%, or more than 70%, preferably more than 80% or 85%, and most preferably more than 90%, or 95%, or even 98% of the T0 population of viable cells.

Prior to lyopreserving (H), the tissue-protectant mixture may, optionally, be subjected to a storing step (F) in which the tissue-protectant mixture is held at a storage temperature for a period of storage time. For example, without limitation, the storage temperature may be a freezing temperature, such as at or below about 0° C., or below about −50° C., or even below about −80° C. A suitable period of storage time may, without limitation, be from about 60 minutes to about 7 days, or from about 60 minutes to about 14 days, or any period of time therebetween).

Also prior to lyopreserving (H), the tissue-protectant mixture may, optionally, be subjected to an incubating step (G) in which the tissue-protectant mixture is held at an incubation temperature such as an ambient or room temperature (i.e., from about 19° C. to about 25° C., or from about 20° C. to about 23° C.), or a refrigerating temperature (i.e., from above 0° C. to about 18° C., or from about 1° C. to about 15° C., or from about 2° C. to about 10° C., or from about 2° C. to about 8° C., or from about 2° C. to about 6° C.), or a warming temperature (i.e., from about 26° C. to about 40° C., or from about 26° C. to about 35° C., or from about 26° C. to about 30° C., or from about 28° C. to about 40° C.). The incubating step (G) may, for example without limitation, be advantageously performed during or after the step of (E) contacting the processed tissue sample with one or more protectants and before any optional step of (F) storing the tissue-protectant mixture, for a period of storage time, at a storage temperature. Without wishing to be limited by theory, it is believed that performing the incubating step (G) wherein the tissue-protectant mixture is stored at lower temperature (e.g., from about 2° C. to about 8° C., or from about 2° C. to about 6° C.) may improve and increase viability of the tissue and cells therein, as well as accelerating or otherwise assisting with uptake of protectants (e.g., non-permeating protectants) by the tissue and cells therein.

A suitable period of incubation time may, without limitation, be from greater than zero seconds to about 48 hours, such as at least about 20 minutes, or at least about 60 minutes, or at least about 120 minutes, or at least about 180 seconds, or at least 240 minutes, or at least 6 hours, or at least 10 hours, or any value between zero and about 48 hours. In some embodiments, such as those in which the tissue sample is a bone tissue sample, the period of incubating time may, without limitation, be at least about 20 minutes, or at least about 30 minutes, or at least about 60 minutes, or at least about 120 minutes, or at least about 180 minutes, or at least 240 minutes, or any value between zero and about 6 hours.

It is noted that any one or more of the contacting step (E), storing step (F), and incubating step (G) may be performed under vacuum, either constant, variable or pulsing, or at pressures greater than atmospheric or ambient pressures. Without being limited by theory, varying the pressures at which these steps are performed may also accelerate or otherwise assisting with the uptake of protectants (e.g., non-permeating protectants) by the tissue sample or cells therein, and/or may enhance or increase viability of the tissue or cells therein.

Additionally, in some embodiments, either of the storing (F) and incubating (G) steps may include contacting the tissue sample, or the tissue-protectant mixture, with one or more storage media, basal media, preservatives, or priming media which includes, without limitation, one or more of: amino acids, glucose, salts, vitamins, or other nutrients that promote for cell survival, and cell-preservative components. In some embodiments, the tissue sample may be staged in such storage media, basal media, preservatives, or priming media to increase cell viability. Additionally, cell viability may be enhanced through the inclusion of use known apoptosis inhibitors, including without limitation, dexamethasone, 3-aminobenzamide, aurintricarboxylic acid, sodium orthovanadate, and combinations thereof. Such staging, storing or incubating may occur during one or more physical and chemical processes performed to accomplish the step (D) of modifying size, shape or other physical characteristics of the tissue sample. Use of cooled storage or staging media may be advantageous if applied to the tissue sample after cutting, slicing and similar techniques to decrease the temperature on the cut surface of the tissue sample.

The preserved tissue forms may further include or be combined with one or more additional components, either prior to, during or after the lyopreserving step (H). Such additional components include, without limitation: mineralized or demineralized bone matrix (cortical or cancellous) in the form of chips, particles, fibers, powder, sheets, chunks, pieces, geometric shapes, etc.; decellularized tissue matrix in the form of chips, particles, fibers, powder, sheets, chunks, pieces, geometric shapes, etc.; other processed or unprocessed tissue forms (whether viable or not) (e.g., cartilage allograft matrix (CAM), lyophilized decellularized delipidized adipose-derived matrix, cryopreserved matrices, etc.); a preservative; a biologically compatible fluid; exogenous cells, viruses, growth factors, proteins, or other biologically active substances; a preservative; an antioxidant; a pharmaceutically active compound; nutritional substances or media; rheology modifiers; crosslinking agents; pH modifiers (buffers); polymers (natural, synthetic, or both); biologically inert excipients (e.g., calcium carbonate, starch, cellulose, glycol, glycerin, mineral stearates, etc.).

In some embodiments, the preserved tissue form further includes one or more natural or synthetic polymers which may be biodegradable and present in proportions selected to provide preserved tissue grafts having various preferred rates of degradation and resorption of the graft or portions thereof. Suitable synthetic polymers include, but are not limited to, bioabsorbable polymers such as polylactic acid (PLA), polyglycolic acid (PGA), polylactic-coglycolide acid (PLGA), and other polyhydroxyacids, polycaprolactones, polycarbonates, polyamides, polyanhydrides, polyamino acids, polyortho esters, polyacetals, degradable polycyanoacrylates and degradable polyurethanes, as well as a polylactide-coglycolide (PLAGA) polymer or a polyethylene glycol-PLAGA copolymer. Examples of natural polymers include, but are not limited to, proteins such as albumin, collagen, fibrin, hyaluronic acid and its derivatives, naturally occurring polyamino acids, and polysaccharides such as alginate, heparin, and other naturally occurring biodegradable polymers of sugar units. The polymeric blend may also include without limitation polycarbonates, polyfumarates, and caprolactones.

The method for producing a preserved tissue form may further comprise an optional step of sterilizing the processed tissue sample or the tissue form comprising the processed tissue sample, either before, during or after the lyopreserving step (H). In some embodiments, the sterilizing step may be performed using one or more techniques including, without limitation: contacting with chemical sterilants, such as without limitation ethylene oxide, highly acidic solutions, highly basic solutions, and the like; and exposure to gamma irradiation, electron beam (E-beam) irradiation, microwave energy, and the like; and filtering with a filter, such as a 0.2 um filter; as will be familiar to persons of ordinary skill. In some embodiments, the sterilizing step may be performed by adding one or more sterilizing agents to the processed tissue sample and/or the preserved tissue form. In some embodiments, sterilizing is performed by terminal sterilization (i.e., performed after the lyopreserving step (H) and after the tissue form has been placed and sealed in a package). For terminally sterilized tissue forms, the protectant could also contain a free radical scavenger or other radio-protectant thus limiting or preventing cell death.

After performing the step of lyopreserving (H) one or more processed tissue samples, the resulting preserved tissue form may be packaged in sterile packaging which may include multiple layers or components. Primary packaging for the tissue may consist of just a single container (jar, vial, bottle, pouch, syringe, cannula, tube, etc.) for both storage media and preserved tissue form, or multiple components such as one container for storage media and preserved tissue form having viable cells, and at least one other container for any non-viable tissue components, and other optional additional components. Packaging components or layers intended to contain the preserved tissue form, with or without storage or other media, should include a moisture barrier for preventing or minimizing moisture (water or other fluid) from entering or escaping from the packaging after sealing with the preserved tissue form therein. In some embodiments, a processed tissue sample may be placed in a first packaging component and subjected to the lyopreserving step (H) while in the first packaging component which is configured and/or made of material which is sufficiently permeable to allow moisture to escape during the lyopreserving step (H). In such embodiments, after lyopreserving, the first packaging component and preserved tissue sample therein may be placed into a second packaging component, which may be configured and/or made of material which includes a moisture barrier for preventing or minimizing moisture (water or other fluid) from entering or escaping from the packaging after sealing with the preserved tissue form therein. In some embodiments, the components of the preserved tissue form, including a processed tissue sample, may be contacted with one or more preservatives (in solution or not) in packaging and then (H) lyopreserved while packaged. Packaging suitable for use during lyophilization is designed, configured and/or made of materials so as to be compatible with the types of tissue, cells, protectants and other components present in the tissue sample or tissue form to be preserved. Furthermore, packaging suitable for containing preserved tissue samples or preserved tissue forms containing same should be capable of preventing cross-contamination between tissue samples derived from different donors and also provide a sterile barrier if needed during lyopreserving (H). Suitable packaging may also include components which facilitate rehydration, handling of the tissue form (such as picking up with forceps, laying flat, etc.), or other preparation of the tissue form such as cutting, perforating, addition of other ingredients such as antibiotics, etc.

Lyopreserving (H) the tissue sample may facilitate molding or otherwise forming the tissue sample into an intentional predetermined shape which may, for example, facilitate end use or delivery to a surgical site or wound site of a subject, as well as providing other physical characteristics such as porosity which may enhance and/or accelerate rehydration.

Clinical applications of the preserved tissue forms described and contemplated herein include use as a surgical graft for the treatment, repair or regeneration of tissue. For example, without limitation, when the preserved tissue forms are derived from tissue samples comprising bone, they will be useful for repair or treatment of bone defects. The preserved tissue forms comprising bone are also suitable for use in orthopedic surgery to promote bone fusion where needed such as, for example without limitation, intervertebral spinal fusion, posterolateral spinal fusion, long bone fusion, or anywhere bone fusion or bony defect filling is required. The preserved tissue forms provide an advantage over previously known cryopreserved cell-containing bone grafts which maintain long-term cell viability (up to five years), but also require storage at temperatures which are at or well below freezing, e.g., −80° C., until use. Accordingly, such cryopreserved bone grafts must be thawed prior to implantation and can also present some logistical issues for surgical centers that do not have the appropriate freezer equipment.

Additionally, when the preserved tissue forms are derived from tissue samples comprising cartilage, they will be useful, for example without limitation, to promote repair and/or regeneration of damaged or worn cartilage where needed such as, for example without limitation, in filling of chondral defects, in filling of osteochondral defects, or in conjunction with other cartilage repair technologies such as, without limitation, microfracture, osteoarticular transfer system (OATS) surgery, and autologous chondrocyte implantation (ACI). Preserved tissue forms derived from tissue samples comprising cartilage would be useful, for example without limitation, in the treatment of joints such as knees, hips, shoulders, elbows, wrists, ankles, knuckles, etc.

When the preserved tissue forms are derived from tissue samples comprising one or more placental tissues (e.g., amnion, chorion, umbilical cord, amniochorion, Wharton's Jelly, etc.), they will be useful, for example without limitation, for use as a wound or tissue covering to treat/repair ulcers, burns, or other wounds; or to treat/repair interior wounds such as tunneling wounds or surgical wounds.

Exemplary methods for using a preserved tissue form prepared as described above, for repairing or reconstructing tissue in a subject, generally comprise:
providing a preserved tissue form comprising one or more processed tissue samples which have been lyopreserved in the presence of one or more protectants and stored at temperatures above freezing;
rehydrating the preserved tissue form by combining (e.g., by mixing, attiring, etc.) the preserved tissue form with a biologically compatible fluid such as, without limitation, saline, blood, bone marrow aspirate (BMA), platelet rich plasma (PRP), and the like, to produce a rehydrated tissue form; and
treating living tissue of a subject by placing the rehydrated tissue form on or in a subject, proximate, adjacent or implanted within the living tissue of the subject.

The ability of the presently described preserved tissue forms, which contain a population of viable cells, to be stored at room-temperature or refrigeration temperatures provides increased efficiencies for both end users and tissue processors. Such preserved tissue forms provide improved ease-of-use for the user by removing the need to thaw and also offers added convenience to those users who are likely to otherwise have to use the graft shortly after receipt and/or lack the appropriate storage facilities for frozen tissue. For tissue processors, non-frozen storage eliminates the need for costly storage resources (dry ice, liquid nitrogen, tanks and freezers, etc.) and removes certain limitations on tissue shipment (limit on shipment time and size due to dry ice). Additional potential uses of these preserved tissue forms includes, without limitation, implanting in extremities for fusion of an ankle, a foot, a wrist, a hand, etc.

In some exemplary embodiments, the preserved tissue forms comprise bone tissue, such as cancellous bone, cortical bone or a combination of both. Such embodiments will now be described in further detail, with the understanding that the presently described and contemplated preserved tissue forms are not limited to those which include bone tissue, but rather include preserved tissue forms which comprise other tissue types as listed above (e.g., adipose, cartilage, amnion, chorion, etc.) and contain a population of viable cells which remain viable after storage at temperatures above freezing and rehydration.

In an exemplary embodiment, the preserved tissue form comprises processed bone tissue derived from cancellous bone samples and containing a population of endogenous viable cells. Optionally, demineralized cortical bone fibers or other allograft materials may be added.

As will be recognized by persons of ordinary skill, the parameters and performance of one or more of the steps of the foregoing method for producing a processed tissue sample and a preserved tissue form comprising same may be modified, selected and optimized depending on the type of tissue sample being processed so as to preserve and retain the beneficial bioactive properties inherent to each tissue type. Descriptions of such specific exemplary embodiments will now be provided in connection with processing bone, cartilage and placental tissue types, with the understanding that these descriptions are not limiting and other variations and modifications to the steps of the method described herein are possible to optimize the processing of other tissue types.

Specifically, processed viable cancellous bone tissue is produced by contacting a bone tissue sample with one or more protectants and then lyopreserving the bone tissue sample and one or more protectants to produce the processed bone tissue which remains viable even after storage at room temperature for extended periods of time. Stored under these conditions, viable cells including osteoblasts, osteoprogenitor cells, and mesenchymal stem cells in the preserved tissue form will remain viable for a period of at least 14 days, or at least 28 days, and preferably greater than 90 days after lyopreserving. An exemplary method for producing such a preserved tissue form comprises the steps of:
(A) Recovering or receiving a recovered cancellous bone tissue sample (for example, cancellous bone used in the graft may be recovered from the hemi-pelvis, humeral head, or other cancellous bone sites containing viable bone-forming cells or mesenchymal stem cells);
(B) Cleaning the cancellous bone tissue sample;
   1. Debriding the cancellous bone tissue sample to remove soft tissue
   2. Rinsing the cancellous bone tissue sample with buffered saline to remove blood and lipids;
(C) Disinfecting the cancellous bone tissue sample;
   1. Rinsing the cancellous bone tissue sample with peracetic acid, mild surfactant, and buffered saline to remove existing bioburden;
(D) Modifying one or more of the size, shape and other physical characteristics of the cancellous bone tissue sample by applying one or more physical treatments;
   1. Cutting the cancellous bone tissue sample to form cancellous blocks;
   2. Optionally, storing the cancellous blocks in preservative or priming media to maintain cell viability (rinse and drain before further processing);
   3. Milling the cancellous bone blocks to form cancellous bone tissue granules;
      a. Disinfecting (C) may be performed particularly efficiently and effectively after one or more size and/or shape modifying steps (D) are performed on bone tissue samples, for examples using peracetic acid;
(E) Contacting/adding cancellous bone tissue granules to a protectant solution which contains one or more protectants dissolved in PBS or other biocompatible fluid to form a cancellous bone tissue-protectant mixture;
(F) Optionally, storing the cancellous bone tissue-protectant mixture in a freezer, such as at a temperature of about −80° C., until lyopreserving; and
(H) Lyopreserving the cancellous bone tissue-protectant mixture to produce a preserved bone tissue form, by performing the following process;
   1. if not already frozen in storage, freezing the cancellous bone tissue-protectant mixture at a rate of from 0.1° C./minute to 2° C./minute; and
   2. drying the frozen cancellous bone tissue-protectant mixture
      a. at either a constant temperature, or at a varied temperature in a range of either from −50° C. to −15° C., or from −15° C. to 25° C., preferably in a range of from 0° C. to 25° C.;
      b. at a pressure of from 0.013 kPa to 0.13 kPa (i.e., from 100 mTorr to 1000 mTorr); and
      c. for a period of time of at least 60 minutes, or at least 180 minutes, or at least 180 minutes, or at least 5 hours, or at least 300 minutes, or at least 600 minutes, or at least 900 minutes, or at least 1200 minutes, or at least 1800 minutes.

The preserved bone tissue form produced by the above described method comprises processed cancellous bone tissue sample and may be stored at temperatures above freezing (e.g., refrigerated or room temperature), for at least 14 days, or at least 28 days, or at least 56 days, or at least 70 days, and preferably for at least 90 days, at least 180 days, at least 365 days, at least 1 year, 2 years, 3 years, or even longer, and still contain viable endogenous cells. More particularly, the preserved bone tissue form comprising processed cancellous bone tissue may be stored at room temperatures (i.e., from about 19° C. to about 25° C.), or refrigeration temperatures (i.e., from greater than 0° C. to about 10° C.), or intermediate temperatures (i.e., from greater than 10° C. to less than about 19° C.). Viable cells in the preserved bone tissue form remain viable after such storage and after rehydration in saline, blood, bone marrow aspirate, PRP or other solution/compound.

All tissue processing steps are performed aseptically. At least one or more of the processing steps are designed to remove immunoreactive elements from the tissue sample, which is demonstrated to not elicit an immune response using an MLR assay, by methods known and understood by persons of ordinary skill in the relevant art. All selected preservation and protectant agents are biologically compatible.

In another exemplary embodiment, an additional component is prepared and added to the bone tissue sample prior to the steps of (E) contacting with one or more protectants and (H) lyopreserving, to produce a preserved tissue form comprising processed cancellous bone tissue and processed cortical bone tissue, as follows. The steps of (A) recovering/receiving, (B) cleaning and (D) modifying the size and shape of a cancellous bone tissue sample to form cancellous bone tissue granules, as described above, are performed. Separately, and possibly but not necessarily concurrently, the following additional steps are performed:
(AA) recovering or receiving recovered a cortical bone tissue sample;
(BB) modifying the size, shape and physical characteristics of the cortical bone tissue sample, by the following steps:
   1. cutting the cortical bone tissue sample to form smaller cortical bone pieces;
   2. milling one or more cortical bone pieces into cortical bone fibers;
   3. demineralizing the cortical bone fibers by methods known now or in the future to persons of ordinary skill in the relevant art; and
(X1) combining, by mixing, stirring, etc., the cancellous bone tissue granules and demineralized cortical bone fibers in a desired ratio (for example without limitation, 70 wt % cancellous bone tissue granules and 30 wt % demineralized cortical bone fibers, or 60 wt % cancellous bone tissue granules and 40 wt % demineralized cortical bone fibers, or 50 wt % cancellous bone tissue granules and 50 wt % demineralized cortical bone fibers, or 40 wt % cancellous bone tissue granules and 60 wt % demineralized cortical bone fibers or 30 wt % cancellous bone tissue granules and 70 wt % demineralized cortical bone fibers based on the total weight of the bone granules and bone fibers) to produce a bone tissue mixture.

Generally, cortical bone samples are recovered from a donor's femur, tibia, humerus, radius, ulna, and fibula, or other suitable long bones. The long bones are first stripped of soft tissue and the shaft cores are cleared of any cancellous bone. The cortical shafts are cleaned using detergents/surfactants to remove residual blood and lipids, then cut into cross-sectional segments of the appropriate length for milling. Milling of the shaft cross-sections results in elongated fibers of cortical bone. Following demineralization in dilute acid, calcium content of the bone fibers is reduced and the cortical bone fibers become putty-like in handling.

After the combining step (X1) in which the cancellous bone granules and cortical bone fibers are mixed in a desired ratio, the remaining method steps to complete production of a preserved bone tissue forms are analogous to those described above for the cancellous-only tissue form. More particularly, this exemplary embodiment further comprises the following steps:
(E) Contacting/adding the bone tissue mixture to a protectant solution which contains one or more protectants dissolved in PBS or other biocompatible fluid to form a bone tissue-protectant mixture;
(F) Optionally, storing the bone tissue-protectant mixture in a freezer, such as at a temperature of about −80° C., until lyopreserving;
(H) Lyopreserving the bone tissue-protectant mixture to produce a preserved bone tissue form, by performing exemplary process delineated above in connection with the cancellous-only tissue form, including;
   1. freezing the bone tissue-protectant mixture;
   2. drying the frozen cancellous bone tissue-protectant mixture
      a. at either a constant temperature, or at a varied temperature; and
      b. at a pressure of from 0.013 kPa to 0.13 kPa.

The preserved bone tissue form produced by the foregoing method may be described as follows:
Allograft Bone:
   40 wt % Mineralized Cancellous Bone Granules (average size=425 μm to 4 mm) containing viable endogenous cells, based on the total weight of the bone granules and bone fibers;
   60 wt % Demineralized Cortical Bone Fibers, based on the total weight of the bone granules and bone fibers (e.g., 50 wt % of fibers having thickness of about 80 μm, and the other 50 wt % of fibers having thickness of about 150 μm, based on the total weight of the Demineralized Cortical Bone Fibers),
   the wt % based on the total weight of the bone granules and bone fibers.
Lyoprotectant solution
   Tissue grafts are stored in a sealed, sterile container at a temperature above freezing (e.g., refrigerated or room temp).

In an exemplary embodiment, the preserved tissue form comprises processed cartilage tissue derived from cartilage samples and containing a population of endogenous viable cells. The type of cartilage tissue suitable for use in the presently described preserved tissue forms and methods for making and using them is not particularly limited. The cartilage tissue may be any type, including without limitation, articular cartilage (such as recovered from condyles, etc.), costal cartilage (such as recovered from anterior ends of one or more ribs), fibrocartilage (such as recovered from intervertebral disks), elastic cartilage (such as recovered from ears), other types of cartilage, and combinations thereof. The cartilage tissue, for example, may be obtained from osteochondral grafts recovered from one or more donors. Osteochondral grafts typically include a bone portion and a cartilage portion which may, but does not have to, be a condyle or hemicondyle. Suitable osteochondral grafts from which cartilage may be recovered include, without limitation, femurs (distal and proximal), talus, and patella. Although smaller, the patella tends to provide more cartilage than femurs because the layer of cartilage on the patella tends to be much thicker.

The cartilage samples, processed cartilage tissue, and preserved cartilage tissue forms comprising processed cartilage tissue, may have any size and shape suitable for the intended use and placement of the resulting preserved tissue form comprising the processed cartilage tissue, including, without limitation, particles, strips, chunks, pieces, blocks, sheets, slivers, ribbons, branched and unbranched elongated elements, filaments, fibers, three dimensional geometric shapes such as symmetric and asymmetric spheres, regular and irregular polyhedrons, cones, pyramids, other three dimensional forms having one or more planar or curved surfaces, and irregular three dimensional forms.

In some embodiments, the preserved tissue form may further comprise cartilage allograft matrix (CAM) such as non-viable freezer-milled cartilage particles, or other materials. In some embodiments, such a preserved tissue form comprising processed cartilage may further comprise, as an additional component, a population of cells selected from: autogenic viable cells isolated from a recipient's (patient's) own tissue, non-immunogenic allogenic viable cells (e.g., cells isolated from suitable allogenic sources such as allogenic cartilage), isolated viable cells which have been differentiated for cartilage, bone, etc. (e.g., added to tissue form prior to lyopreserving (H)), “dry cells” (which have been lyophilized, either in contact with a protectant or not, separately from the processed tissue and preserved tissue form).

Specifically, in an exemplary embodiment, a processed viable cartilage tissue is produced by contacting a cartilage tissue sample with one or more protectants and then lyopreserving the cartilage tissue sample and one or more protectants to produce a processed cartilage tissue which remains viable even after storage at room temperature for extended periods of time. Stored under these conditions, viable cells including chondrocytes, chondroblasts, cartilage progenitor cells, and mesenchymal stem cells in the preserved tissue form will remain viable for a period of at least 14 days, or at least 28 days, and preferably greater than 90 days after lyopreserving. An exemplary method for producing such a preserved tissue form comprises the steps of:
(A) Recovering or receiving a recovered a cartilage tissue sample may begin with recovering or receiving a recovered bone tissue sample which typically includes a bone portion and a cartilage portion, where the cartilage portion further contains one or more endogenous viable cells including chondrocytes, cartilage-forming cells, and mesenchymal stem cells (for example, a femur having at least one intact condyle with a cartilage surface which contains viable cells);
(B1) Cleaning the bone tissue sample by debriding the bone tissue sample to remove soft tissue;
(D1) Modifying the size and shape of the bone tissue sample by cutting the bone tissue sample into at least two pieces, each comprising a bone portion and a cartilage portion (keep pieces moist by contacting with phosphate buffered saline (PBS));
(B2) Cleaning the resulting pieces by rinsing them one or more times (e.g., four times) with buffered saline or similar biocompatible fluid (rinsed pieces may be held in PBS for up to 8 hours prior to further processing steps);
(C) Disinfecting the pieces by contacting them with one or more antibiotics (e.g., place them in a container with a mixture of penicillin, streptomycin, amphotericin B);
   1. for example, an ambient storage media containing one or more antibiotics may be used for concurrently storing and disinfecting the pieces, where such an ambient storage media may contain:
      a. DMEM,
      b. Antibiotic-Antimycotic (penicillin/streptomycin/amphotericin B),
      c. Non-Essential Amino Acid (NEAA) cell culture supplement,
      d. L-glutamine,
      e. insulin-transferrin-selenium (ITS)
      f. L-ascorbic acid (Vitamin C), and
      g. dexamethasone;
   2. In embodiments where cartilage tissue samples are (A) recovered or received “en block,” or as one or more whole tissue sections, and not in an ambient storage media or other stabilizing media, the cartilage tissue samples may be subjected to a disinfecting (C) step (for example, by rinsing with PBS, mild surfactant, and peracetic acid and water to remove existing bioburden) prior to or during one or more cleaning (B) and modifying (D) steps
(D2) Modifying the size and shape of the cartilage portion of each piece by grating or shaving with a grater to produce viable cartilage fibers containing one or more types of viable cells (as noted above);
   1. Mount the bone portion of the piece in a vise (avoid damage to cartilage portion),
   2. Using the grater (e.g., an OXO® brand steel grater Model #50581), grate the cartilage portion of the shaft (e.g., until the cartilage has been removed from the bone portion),
   3. Rinse the grater intermittently (e.g., after every 4 strokes) with a biocompatible fluid (e.g., the ambient storage media which contains antibiotics) to separate the resulting viable cartilage fibers from the grater and collect them in the biocompatible fluid,
   4. Separate the viable cartilage fibers from the biocompatible fluid, e.g., by using a mesh or similar filtering device to drain off the biocompatible fluid and retain the viable cartilage fibers,
(B3) Cleaning and wetting the viable cartilage fibers by rinsing them one or more times (e.g., twice) with PBS or similar biocompatible fluid (gentle agitation during the second rinse with fresh PBS ensures all viable cartilage fibers are wetted while minimizing damage),
   1. when the above-described ambient storage media is used to collect the viable cartilage fibers, it must be rinsed off the viable cartilage fibers within three hours from when the viable cartilage fibers are initially soaked in the ambient storage media, therefore, so this rinsing step must generally be performed within three hours of when the grating is commenced (step (D2)));
(E) Contacting/adding the viable cartilage fibers to a protectant solution which contains one or more protectants dissolved in a biocompatible fluid (e.g., PBS or other biocompatible fluid) to form a cartilage tissue-protectant mixture;
   1. an exemplary protectant solution contains about 0.4 M trehalose and 0.4 mM EGCG, dissolved in 1×PBS,
   2. the protectant solution may, optionally, also contain one or more additional ingredients such as sugar alcohols, antioxidants, and other additives,
   3. contacting the viable cartilage fibers with the protectant solution by be accomplished, for example, by combining a 0.75-0.85 gram quantity of cartilage fibers with about 4-6 milliliters (mL) of protectant solution in a 30 mL jar,
(F) Optionally, storing the cartilage tissue-protectant mixture in a freezer, such as at a temperature of about −80° C., until lyopreserving; and
(H) Lyopreserving the cartilage tissue-protectant mixture to produce a preserved cartilage tissue form (comprising viable cartilage fibers), by performing the following process;
   1. if not already frozen in storage, freezing the cartilage tissue-protectant mixture at a rate of from 0.1° C./minute to 2° C./minute; and
   2. drying the frozen cartilage tissue-protectant mixture
      a. at either a constant temperature, or at a varied temperature in a range of either from −50° C. to −10° C., or from −10° C. to 25° C., preferably in a range of from 0° C. to 25° C.;
      b. at a pressure of from 0.013 kPa to 0.13 kPa (i.e., from 100 mTorr to 1000 mTorr); and
      c. for a period of time of, for example, at least 60 minutes, or at least 180 minutes, or at least 240 minutes, or at least 5 hours, or at least 400 minutes, or at least 600 minutes, or at least 900 minutes, or at least 1200 minutes, or at least 1500 minutes, or at least 1800 minutes, or any period of time up to about 35 hours.

The preserved tissue form comprising a processed cartilage tissue sample (i.e., the preserved cartilage tissue form) which is produced by the above described method may be stored at temperatures above freezing (e.g., refrigerated or room temperature), for at least 14 days, or at least 28 days, or at least 56 days, or at least 70 days, and preferably for at least 90 days, at least 180 days, at least 365 days, at least 1 year, 2 years, 3 years, or even longer, and still contain viable endogenous cells. More particularly, the preserved cartilage tissue form comprising processed cartilage tissue may be stored at room temperatures (i.e., from about 19° C. to about 25° C.), or refrigeration temperatures (i.e., from greater than 0° C. to about 10° C.), or intermediate temperatures (i.e., from greater than 10° C. to less than about 19° C.). Viable endogenous cells in the preserved cartilage tissue form remain viable after such storage and after rehydration in saline, blood, bone marrow aspirate, PRP or other solution/compound.

The cartilage processing method described above produces viable cartilage fibers which contain viable mesenchymal stem cells, viable chondrocytes, growth factors and matrix proteins, all of which are endogenous and were initially present in the original cartilage tissue sample (i.e., bone and cartilage). Additionally, the viable cartilage fibers are cohesive with one another and, after shaping and implantation into a cartilage void or defect (with or without additional components), tend to remain in the cartilage void or defect, even when the cartilage void or defect and surrounding region are rinsed or irrigated with biocompatible fluid, or gently wiped with a finger, sponge or other suitable device.

The preserved tissue form comprising processed cartilage tissue (i.e., a preserved cartilage tissue form) may further comprise one or more additional components as described above (e.g., CAM, fibrin glue, etc.). The preserved tissue form comprising processed cartilage tissue may be packaged, such as in a foil pouch, with or without a moisture barrier. In some embodiments, the components of the preserved tissue form, including a processed cartilage tissue sample, may be contacted with one or more preservatives (in solution or not) in packaging and then (H) lyopreserved while packaged.

All tissue processing steps are performed aseptically. At least one or more of the processing steps are designed to remove immunoreactive elements from the tissue sample, which is demonstrated to not elicit an immune response using an MLR assay, by methods known and understood by persons of ordinary skill in the relevant art. All selected preservation and protectant agents are biologically compatible.

A preserved cartilage tissue form comprising a processed cartilage tissue as described herein may be implanted into a recipient (patient, human or non-human) with damaged or otherwise inadequate cartilage as a result, for example without limitation, of disease or naturally or surgically created cartilage voids. In some embodiments, the preserved cartilage tissue form further comprises CAM and, when rehydrated with a biocompatible fluid, forms a putty and is moldable, manually or otherwise (e.g., using a mold or other container), to facilitate filling any cartilage void in need of repair. For example, in some embodiments, processed (viable) cartilage fibers and one or more protectants may be placed in a mold (i.e., a container having a cavity of desired shape and size), and lyophilized together and then packaged in a kit to be used in a syringe, cannula, or other delivery device. In some embodiments, one or more additional components such as, without limitation, CAM, may be combined with the processed cartilage fibers in the mold and lyophilized together to form a preserved cartilage tissue form having a predetermined size and shape (i.e., the aforesaid desired size and shape of the cavity). In some embodiments, the processed cartilage fibers are lyophilized (in a mold or not), then packaged in a kit, with one or more additional components such as, without limitation, CAM, also packaged in the kit, but separate from the processed cartilage fibers, with the intent that the processed cartilage fibers and additional components are combined with one another and, optionally, a biocompatible fluid, at the time of use and implantation into (or onto) a subject (patient). Such preserved cartilage tissue forms (i.e., the processed cartilage fibers) contain viable chondrocytes, chondrogenic growth factors, and extracellular matrix components which promote repair and healing of the cartilage void. More particularly, the preserved cartilage tissue form is expected to synthesize, and/or promote deposition of, hyaline cartilage and proteoglycans.

In an exemplary embodiment, the preserved tissue form comprises processed placental tissue derived from placental samples and containing a population of endogenous viable cells. In some embodiments, such a preserved tissue form comprising processed placental tissue, may further comprise, as an additional component, a population of cells selected from: autogenic viable cells isolated from a recipient's (patient's) own tissue, non-immunogenic allogenic viable cells (e.g., cells isolated from suitable allogenic sources such as allogenic cartilage), isolated viable cells which have been differentiated, “dry cells” (which have been lyophilized, either in contact with a protectant or not, separately from the processed tissue and preserved tissue form).

Specifically, in an exemplary embodiment, a processed viable placental tissue comprising processed amnion tissue and processed chorion tissue, is produced by contacting an amnion tissue sample and a chorion tissue sample with one or more protectants and then lyopreserving the tissue samples and the one or more protectants to produce the processed placental tissue which remains viable even after storage at room temperature for extended periods of time. Stored under these conditions, viable cells, including one or more types of viable cells including epithelial cells and stromal cells (such as fibroblasts and mesenchymal stem cells) in the preserved tissue form, will remain viable for a period of at least 14 days, or at least 28 days, and preferably greater than 90 days after lyopreserving. An exemplary method for producing such a preserved tissue form comprises the steps of:
(A) Recovering or receiving a recovered placenta sample which includes at least an amnion membrane and a chorion membrane;
   a. Optionally, umbilical cord may also be included.
(B1) Separating the amnion and chorion membranes from one another and other portions of the placenta sample, by:
   1. manually peeling the amnion and chorion membranes apart,
   2. cutting the amnion membrane free from the umbilical cord, if present,
   3. cutting the chorion membrane free from the placental disk, if present;
   4. Optionally, umbilical cord also may be cut free from the placental disk and then cleaned and processed in a similar manner as the subsequent steps.
(B2) Cleaning and removing blood and blood clots from each of the amnion and chorion membranes by:
   1. manually dabbing each of the amnion and chorion membranes with wetted wipes,
   2. contacting the chorion membrane (one or more times), preferably with agitation, with an RBC lysis solution containing ingredients which lyse red blood cells and break up blood clots (e.g., a buffer solution comprising streptokinase [commercially available as MP BIOMEDICALS brand from Thermo Fisher Scientific of Waltham, Massachusetts, U.S.A.], and a red blood cell lysis buffer [commercially available from Sigma Aldrich of St. Louis, Missouri, U.S.A.]),
   3. separating the chorion membrane from the RBC lysis solution, rinsing the chorion membrane (one or more times) with a biocompatible fluid (e.g., HBSS) and dabbing with wetted wipes and/or sterile cotton applicators to remove additional blood and blood clots,
   4. soaking the amnion membrane (one or more times) in a biocompatible buffer solution (e.g., HBSS) and dabbing with wetted wipes (e.g., soak and wipe twice for about 10 to about 20 minutes each time);
(C) Disinfecting the amnion and chorion membranes (together or separately) by contacting them with one or more antibiotics, preferably with agitation (e.g., place them in a container with a mixture of vancomycin, gentamicin, amphotericin B and agitate for about 30 to about 60 minutes);
(B3) Cleaning the amnion and chorion membranes to remove the one or more antibiotics by soaking and rinsing, preferably with agitation (one or more times) with a biocompatible fluid (e.g., perform a first rinse in a flask with HBSS and shaking at 65 rpm for 5-10 min, repeat for a second rinse (preferably with fresh HBSS), and perform a third rinse in a flask with HBSS (preferably fresh) and shaking at 65 rpm for 30-40 min),
(B4) maintaining hydration of the amnion and amnion and chorion membranes by placing and holding them (together or separate) in a biocompatible fluid (e.g., HBSS);
   1. optionally, further dabbing and wiping with wetted wipes and/or sterile cotton applicators may be performed to remove any residual blood clots or remnants;
   2. optionally, separating and removing the trophoblast layer from the chorion membrane by gently peeling, scraping, or sloughing portions of the trophoblast layer using hands/fingers, a scraper, spatula or similar devices, wetted wipes, or a combination of thereof;
(D1) Modifying the size and shape of the amnion and chorion membranes by laying each membrane flat and cutting with a blade or similar device,
   1. for example, arranging the amnion and chorion membranes together, by placing the amnion membrane on a backing material, optionally with the epithelial side facing down and in contact with the backing material, and placing the chorion membrane on top of the amnion membrane, optionally with the trophoblast side (with or without the trophoblast layer present) facing up and not in contact with the amnion membrane, then cutting the layered amnion and chorion membranes into smaller amnion-chorion pieces of desired size and shape (still with backing material), and placing the cut pieces back into the biocompatible fluid (e.g., HBSS) to maintain hydration of the cut pieces during the remaining cutting;
   2. optionally, the cut amnion-chorion pieces may be soaked or stored in a biocompatible fluid (e.g., HBSS) to maintain hydration of the cut pieces during the remaining cutting, and removed from the biocompatible fluid prior to contacting with one or more protectants;
(E) Contacting/adding the cut amnion-chorion pieces to a protectant solution which contains one or more protectants dissolved in a biocompatible fluid (e.g., HBSS or other culture media) to form an amnion-chorion tissue-protectant mixture;
   1. an exemplary protectant solution contains about 0.5 M trehalose and 4.0 mM EGCG, dissolved in HBSS,
   2. the protectant solution may, optionally, also contain one or more additional ingredients such as sugar alcohols, antioxidants, and other additives,
   3. the contacting time may be from about 15 to about 60 minutes,
   4. the contacting temperature may be from greater than 0° C. to about 10° C.,
(H) Lyopreserving the amnion-chorion tissue-protectant mixture to produce a preserved amnion-chorion tissue form (comprising both processed amnion and processed chorion), by performing the following process;
   1. if not already frozen in storage, freezing the amnion-chorion tissue-protectant mixture at a rate of from 0.1° C./minute to 2° C./minute, optionally with a period of constant temperature following the controlled-rate freezing, for a total freezing time of from about 80 min to about 160 min (e.g., from 120 min to 150 min, or from 60 min to 90 min, or another time period); and
   2. drying the frozen amnion-chorion tissue-protectant mixture
      a. at either a constant temperature, or at a varied temperature in a range of either from −50° C. to less than −10° C., or from −10° C. to 25° C., preferably in a range of from −10° C. to 25° C.;
      b. at a pressure of from 0.013 kPa to 0.13 kPa (i.e., from 100 mTorr to 1000 mTorr); and
      c. for a period of time of at least 60 minutes, or at least 180 minutes, or at least 200 minutes, or at least 300 minutes, or at least 400 minutes, or at least 600 minutes, or at least 900 minutes.

As will be readily recognized by persons of ordinary skill in the relevant art, several modifications and additions may be made to customize and optimize the foregoing method for producing a preserved amnion-chorion tissue form as described herein. For example, in some embodiments, a color indicator, such as phenol red, may be added to the biocompatible fluid to impart a visible color to the amnion and chorion membranes to facilitate visualization during cutting. In some embodiments, to facilitate handling and protect the amnion-chorion pieces from damage, the cut amnion-chorion pieces may be placed into a retainer, netting, mesh, or other type of permeable container after the size modifying step (D) and prior to performing one or more additional processing steps (e.g., contacting with protectants, rinsing, etc.). Furthermore, in some embodiments, the step of (E) contacting the cut amnion-chorion tissue pieces with the protectant solution may include draining the protectant solution after the desired period of contacting time, briefly (about 5 minutes) soaking or rinsing the cut amnion-chorion tissue pieces with a biocompatible fluid (e.g., HBSS); draining excess fluid from the cut amnion-chorion pieces and placing them into one or more breathable pouches (such as without limitation, Tyvek pouches) and sealing the pouch(es), and perform the (H) lyopreserving step while the amnion-chorion pieces are sealed in the pouches. After (H) lyopreserving, the pouches containing lyopreserved amnion-chorion tissue may be placed and sealed in an external packaging material, such as moisture-barrier pouches.

The preserved tissue form comprising a processed amnion-chorion tissue (i.e., the preserved amnion-chorion tissue form) produced by the above described method may be stored at temperatures above freezing (e.g., refrigerated or room temperature), for at least 14 days, or at least 28 days, or at least 56 days, or at least 70 days, and preferably for at least 90 days, at least 180 days, at least 365 days, at least 1 year, 2 years, 3 years, or even longer, and still contain viable endogenous cells. More particularly, the preserved cartilage tissue form comprising processed cartilage tissue may be stored at room temperatures (i.e., from about 19° C. to about 25° C.), or refrigeration temperatures (i.e., from greater than 0° C. to about 10° C.), or intermediate temperatures (i.e., from greater than 10° C. to less than about 19° C.). Viable endogenous cells in the preserved cartilage tissue form remain viable after such storage and after rehydration in saline, blood, bone marrow aspirate, PRP or other solution/compound.

The placenta (e.g., amnion and chorion) processing method described above produces pieces of layered amnion and chorion membranes which contain viable epithelial and stromal cells (such as fibroblasts and mesenchymal stem stems), growth factors and matrix proteins, all of which are endogenous and were initially present in the original placenta tissue sample (i.e., amnion and chorion).

The preserved amnion-chorion tissue form may further comprise one or more additional components as described above (e.g., exogenous cells, other processed tissues or grafts, etc.). The preserved tissue form comprising processed amnion-chorion tissue may be packaged, such as in a pouch, preferably within a moisture barrier package. In some embodiments, the components of the preserved tissue form, including a processed cartilage tissue sample, may be contacted with one or more preservatives (in solution or not) in packaging and then (H) lyopreserved while packaged.

All tissue processing steps are performed aseptically. At least one or more of the processing steps are designed to remove immunoreactive elements from the tissue sample, which is demonstrated to not elicit an immune response using an MLR assay, by methods known and understood by persons of ordinary skill in the relevant art. All selected preservation and protectant agents are biologically compatible.

In another exemplary embodiment, in which the preserved tissue form comprises processed placental tissue derived from placental samples and containing a population of endogenous viable cells, the placental tissue may comprise processed umbilical cord tissue sample. Specifically, a processed viable placental tissue comprising processed umbilical cord tissue may be produced by a method similar to that described above in connection with the processing of amnion and chorion tissues, except that at least some of the steps of (B) cleaning and (D) modifying the size, shape and other physical characteristics will be different due to the physical and other differences between amnion, chorion and umbilical cord.

More particularly, as will be understood by persons of ordinary skill in the relevant art, after (A) recovering or receiving a recovered placenta tissue sample, one or more of several cleaning steps (B) may be performed including, for example without limitation, (B1) cutting and separated the umbilical cord tissue sample from the remaining placental tissues, (B2) separating and removing blood vessels from the umbilical cord tissue sample, and (B3) rinsing the umbilical cord tissue sample with one or more biocompatible fluids to remove unwanted materials (e.g., blood, debris, etc.). The umbilical cord tissue sample may also be subjected to the step of (D) modifying one or more of the size and shape of the umbilical cord tissue sample by cutting the umbilical cord longitudinally and spreading or flattening the umbilical cord into a planar or sheet-like configuration.

Such size and shape modification steps (D) may be followed by one or more additional cleaning steps (B) such as rinsing or soaking with one or more biocompatible fluid. It should be noted that the foregoing steps may be performed in a different order than described above, according to the judgment of persons or ordinary skill in the relevant art. Thereafter, like the method for processing amnion and chorion tissues, the umbilical cord tissue sample is (E) contacted with one or more protectants and then the resulting umbilical cord tissue-protectant mixture will be (H) lyopreserved to produce a processed umbilical cord tissue which remains viable even after storage at room temperature for extended periods of time. Stored under these conditions, viable cells, including one or types of viable cells including epithelial cells and stromal cells (such as fibroblasts and mesenchymal stem cells) in the preserved tissue form, will remain viable for a period of at least 14 days, or at least 28 days, and preferably greater than 90 days after lyopreserving.

Such a processed umbilical cord tissue form may further comprise other processed tissues such as, without limitation, processed amnion, processed chorion, or both, as well as one or more additional components, etc. For example, in some embodiments, such a preserved tissue form comprising processed umbilical cord tissue, may further comprise, as an additional component, a population of cells selected from: autogenic viable cells isolated from a recipient's (patient's) own tissue, non-immunogenic allogenic viable cells (e.g., cells isolated from suitable allogenic sources such as allogenic cartilage), isolated viable cells which have been differentiated, “dry cells” (which have been lyophilized, either in contact with a protectant or not, separately from the processed tissue and preserved tissue form).

It will be understood that the embodiments of the present invention described hereinabove are merely exemplary and that a person skilled in the art may make variations and modifications without departing from the spirit and scope of the invention. All such variations and modifications are intended to be included within the scope of the present invention.

### EXAMPLES

The following descriptions, flowcharts and tables provide exemplary embodiments of methods for making the viable tissue forms from particular recovered tissue types, including human bone, human amnion, and human adipose.

### Characterization Test Methods

The following descriptions provide explanations for each of the test methods employed to produce the characterization data and information provided below.

Analysis and assessment of cell presence and cell viability are measured using the following techniques. A tissue sample is set aside at any point during a processing method at which the presence and/or viability of the cells is desired to be measured, so that cell viability/biological activity of the tissue can be evaluated at that point using commercially available methods. While such suitable methods include, but are not limited to, metabolic assays, such as those involving luciferase, tetrazolium salts (e.g., 3-(4, 5-dimethyl-2-thiazolyl)-2, 5-diphenyl-2H-tetrazolium bromide (MTT), 3-(4,5-dimethylthiazol-2-yl)-5-(3-carboxymethoxyphenyl)-2-(4-sulfophenyl)-2H-tetrazolium (MTS), 2,3-bis-(2-methoxy-4-nitro-5-sulfophenyl)-2H-tetrazolium-5-carboxanilide (XTT), and other water soluble tetrazolium salts (e.g., WST-1, -3, -4, -5, -8, -9, -10, and -11)), live-dead assays, ATP assay, CCK-8 assay and dye exclusion assays such as Trypan Blue, for the presently described and contemplated preserved tissue forms and methods for producing them, metabolic activity is determined by performing an ATP assay, as follows:

A viability assay based on the metabolic activity of a sample is performed by measuring the ATP content in the sample. In an embodiment, the assay is performed by thawing frozen tissue samples (if needed) and then transferring the tissue samples into a rinse basin. The thawed and/or rinsed tissue samples are combined with an assay reagent and incubated on an orbital shaker. After incubation, aliquots of the incubated reagent are transferred into a 96-well plate and raw luminescence values (in RLU), for example, are obtained using a plate reader. A standard curve is prepared by creating serial dilutions of known concentrations of an ATP standard, combining with assay reagent, incubating on an orbital shaker, and then reading the RLU of each dilution. The dilutions' RLUs are plotted against the known concentrations of each serial dilution to generate an equation, which is then used to convert RLU of the tissue samples to concentrations of ATP. Finally, depending on the type of tissue, the tested tissue samples may be rinsed in water or a biologically compatible fluid (such as, without limitation, 5% Dextrose in Lactated Ringer's Solution (DSLR), phosphate buffered saline (PBS), saline, HBSS, etc.), before being placed into pre-weighed weighing pans. The tissue sample may then be dried (e.g., if wet) and weighed, and the concentration of ATP per tissue sample is converted to amount of ATP per weight of tissue sample. Alternatively, the tissue sample may be undried, i.e., weighed in a wet condition, or it may be weighed dry prior to incubation with assay reagent. As will be understood by persons of ordinary skill in the relevant art, whether a tissue sample is wet or dry prior to incubation with assay reagent, and whether a tissue sample is rinsed (and with what type of fluid) prior to drying and weighing, will depend on the type of tissue. For example, bone tissue may be wet prior to incubation with assay reagent and not rinsed or weighed after RLU values are obtained, so that the pre-testing wet weight is used to calculate the amount of ATP per weight of tissue sample. Alternatively, placental (e.g., amnion, chorion, etc.) tissue may be rinsed after obtaining RLU values, then dried and weighed, as explained above. The tissue sample being tested for viable cell population content may be wet or dry and the foregoing ranges for the results of such testing are applicable to both wet and dry tissue samples.

As previously explained, a preserved tissue sample has a pre-lyopreservation population of endogenous viable cells and a post-lyopreservation (i.e., Week 0) population of endogenous viable cells. The post-lyopreservation population of endogenous viable cells is a portion (e.g., a percentage) of the pre-lyopreservation population of endogenous viable cells. Additionally and independently, the post-lyopreservation population of endogenous viable cells is also a portion (e.g., a percentage) of the pre-contact population of endogenous viable cells.

The preserved tissue samples (and preserved tissue forms comprising them) contain populations of endogenous viable cells which are substantially comparable in quantity and proportion to populations of endogenous viable cells contained in cryopreserved tissue forms (and cryopreserved tissue forms comprising them), where the preserved and cryopreserved tissue samples are of the same type of tissue and the populations of endogenous viable cells of each of the preserved and cryopreserved bone tissue samples are measured at the same point of processing or after the same period of time of storage after preservation and cryopreservation. For example, without limitation, a preserved bone tissue sample comprises a population of endogenous viable cells which is substantially comparable, i.e., within ±15%, or within ± any percent between 15% and 1%, to the population of endogenous viable cells contained in a cryopreserved bone tissue sample, wherein both of the populations of endogenous viable cells are any one of: post-contact (i.e., measured after contacting with one or more protectants and after contacting with one or more cryopreservation agents, or “pre-lyopreservation” and “pre-cryopreservation,” respectively) populations of endogenous viable cells, post-lyopreservation and post-cryopreservation populations of endogenous viable cells, respectively (i.e., T0 or Week 0 populations), or retained populations of endogenous viable cells (i.e., measured after a period of storage time). Similarly, for example, without limitation, a preserved amnion tissue sample comprises a population of endogenous viable cells which is substantially comparable, i.e., within ±85%, or within ± any percent between 85% and 1%, to the population of endogenous viable cells contained in a cryopreserved amnion tissue sample, wherein both of the populations of endogenous viable cells are measured at the same point of processing or after the same period of storage time, as explained above. Similarly, for example, without limitation, a preserved cartilage tissue sample comprises a population of endogenous viable cells which is substantially comparable, i.e., within ±50%, or within ±any percent between 50% and 1%, to the population of endogenous viable cells contained in a cryopreserved cartilage tissue sample, wherein both of the populations of endogenous viable cells are measured at the same point of processing or after the same period of storage time, as explained above.

In the following experiments, all tissue samples were human tissues obtained from eligible donors after obtaining written informed consent, and applicable tissue regulations for receipt and disposition of tissues were strictly followed.

Furthermore, each of the following experiments which involved preservation methods in accordance with those described and contemplated above were performed using a “Lyophilizing Apparatus” (or “Lyophilizer”) which were commercially available, at the time of the experiments, under the brand name VirTis® from SP Scientific of Warminster, Pennsylvania, U.S.A. and were capable of the following operating parameters (for example, several of the AdVantage models and a Genesis Pilot model):
Shelf Temperature range of from −40° C. to +25° C.;
Capable of reaching Condenser Temperature of −60° C. or less; and
Condenser Capacity of 3.5 liters or greater, and 2.5 liters or greater capacity over 24 hours of condensation operation.

### Example 1

Preserved Tissue Form Containing Both Viable Cancellous Bone Granules and Demineralized Cortical Bone Fibers with Demonstrated Viability After Lyopreservation and Rehydration

Two experiments were performed and are reported below.

### Experiment 1

In the first experiment, cancellous tissue samples were recovered from a single donor, and processed to provide cancellous granules. Multiple samples of the processed cancellous granules were preserved after contact with protectants in solution, in accordance with the methods described hereinabove, and analyzed for cell viability by measuring ATP. The ATP results reported below are based on the mathematical average of the measured ATP results for all of the aforesaid samples derived from the single donor of Experiment 1.

### Experiment 2

In the second experiment, cancellous tissue samples comprising cancellous bone were recovered from a single donor (different from the donor of Experiment 1) and processed to provide cancellous granules, and cortical bone tissue samples were recovered from the same single donor of Experiment 2 and processed to provide cortical fibers. Multiple samples, each comprising both cancellous granules and cortical fibers, were preserved after contact with protectants in solution, in accordance with the methods described hereinabove, and analyzed for cell viability by measuring ATP. The ATP results reported below are based on the mathematical average of the measured ATP results for all of the aforesaid samples comprising both cancellous granules and cortical fibers and derived from the single donor of Experiment 2.

### A. Protectant Solution Preparation:

1. Protectant solutions were prepared for each of Experiments 1 and 2 by dissolving trehalose and EGCG into PBS at concentrations of 0.4 M and 4 mM, respectively.
2. Following dissolution of the preservative ingredients (i.e., protectants trehalose and EGCG) in media, the solution was sterile filtered using a 0.2 μm membrane filter.

### B. Sample Preparation (Performed in Sterile Hood)

1. Viable cancellous tissue (granules) and demineralized cortical tissue (fibers) were obtained by sterile processing techniques as described hereinabove in connection with processing viable cancellous bone tissue (i.e., for cancellous granules: (A) recovered/received cancellous bone sample, (B) cleaned by debriding and rinsing, (D) modified by (1) cutting and then (3) milling to produce granules of average particle size from 425 μm to 4 mm), followed by (C) disinfected to remove bioburden by rinsing with peracetic acid, mild surfactant, and buffered saline; and for cortical fibers: (AA) recovered/received cortical bone sample, (BB) modified by (1) cutting, (2) milling to produce fibers of average width from 80 um to 150 um and average length from 8 mm to 12 mm), and then (3) demineralizing the fibers to a mineral content of less than 8 wt %).
2. Cancellous granules either alone or with demineralized cortical fibers were packaged and temporarily stored at refrigerated temperature (2-4° C.) for a period of time greater than 0 and up to 90 minutes, until time of use in experiment.
3. In a sterile hood, multiple samples were weighed out into 1 oz. jars, as follows:
   a. Experiment 1: each sample included 0.5 g cancellous granules, and
   b. Experiment 2: each sample included 0.5 g cancellous granules and 0.8 g cortical fibers.
4. 4 mL of sterile protectant solution was added to each of the jars containing tissue samples.
5. Tissue was allowed to incubate with protectant solution at room temperature for 1 hour.
6. After the incubation period, jars containing tissue were placed on an aluminum or stainless steel tray and placed in the Lyopreserving Apparatus.
7. All tissue samples in Experiment 1 were lyopreserved according to the recipe provided in Table 1 below. All tissue samples in Experiment 2 were lyopreserved according to the recipe provided in Table 2 below.

TABLE 1
Experiment 1 Lyophilization Recipe

| | Temperature (° C.) | Time (min) | Vacuum (mTorr) | Ramp/Hold |
|---|---|---|---|---|
| Thermal Treatment | | | | |
| Step 1: Freeze | −30 | 120 | | Ramp |
| Freeze, Condenser, and | | | | |
| Vacuum Parameters | | | | |
| Freeze Temperature (° C.) | −30 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | 0 | 40 | 200 | Ramp |
| Step 2 | 0 | 360 | 200 | Hold |
| Step 3 | 15 | 15 | 200 | Ramp |
| Step 4 | 15 | 525 | 200 | Hold |
| Step 5 | 25 | 15 | 200 | Ramp |
| Step 6 | 25 | 360 | 200 | Hold |

TABLE 2
Experiment 2 Lyophilization Recipe

| | Temperature (° C.) | Time (min) | Vacuum (mTorr) | Ramp/Hold |
|---|---|---|---|---|
| Thermal Treatment | | | | |
| Step 1: Freeze | −30 | 120 | | Ramp |
| Freeze, Condenser, and | | | | |
| Vacuum Parameters | | | | |
| Freeze Temperature (° C.) | −30 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | −15 | 40 | 200 | Ramp |
| Step 2 | −15 | 900 | 200 | Hold |
| Step 3 | 1 | 60 | 200 | Ramp |
| Step 4 | 1 | 500 | 200 | Hold |

Key: “Ramp” indicates variable temperature over the time period indicated (Time (min)).
“Hold” indicates temperature held constant over the time period indicated (Time (min))

### C. Sample Testing

1. Prior to contact with the protectant solution (i.e., prior to step B.4. above), a first set of samples of about 0.5 g of the cancellous tissue granules from Experiment 1, and a second set of samples of 0.5 g the cancellous tissue and 0.8 g of the cortical tissue fibers (total of 1.3 g combined bone tissue) from Experiment 2 were measured and separated, and each of the first and second pre-contact samples was tested per the ATP viability assay procedures described above, which provided “Baseline” (i.e., “pre-contact”) ATP values.
2. Prior to lyopreservation but post-contact and incubation with the protectant solution (i.e., prior to step B6), a first set of samples of about 0.5 g of the cancellous tissue granules from Experiment 1, and a second set of samples of 0.5 g the cancellous tissue and 0.8 g of the cortical tissue fibers (total of 1.3 g combined bone tissue) from Experiment 2 were measured and separated, and each of the first and second post-contact samples was tested per the ATP viability assay procedures described above, which provided “Pre-Lyo” (i.e., “post-contact and incubation”) ATP values.
3. In both of Experiments 1 and 2, following lyopreserving, each of the samples was rehydrated by adding 5 mL of PBS 1× to each jar and letting sit at room temperature for 5-10 minutes (time varied based on experiment).
4. Following the 5-10 minute rehydration period, excess PBS solution was decanted from all jars.
5. 0.5 g of rehydrated cancellous bone tissue (Experiment 1) and 1.3 g of rehydrated combined cancellous bone tissue and cortical bone fibers (Experiment 2) were each weighed out and tested per the ATP viability assay procedures described above, which provided Week 0 ATP values. The ATP data resulting from the foregoing experimental procedure was converted to % of Baseline ATP and % of Pre-Lyo ATP, as reported in Table 3 below, using the following formulas:

$$\% of Baseline ATP = ( Average Week 0 ATP value / Average Baseline ATP value ) \times 100$$

$$\% of Pre - Lyo ATP = ( Average Week 0 ATP value / Average Pre - Lyo ATP value ) \times 100$$

TABLE 3

| | | % of | % of |
|---|---|---|---|
| | | Baseline | Pre-Lyo |
| Experiment # | Description | ATP | ATP |
| 1 | Cancellous granules | 79% | 85% |
| 2 | Cancellous granules | 56% | 93% |
| | with demineralized | | |
| | cortical fibers | | |

### Example 2

### Preparation of Lyopreserved Cancellous Bone Tissue Form (Granules) and Cell Viability After Storage at Room Temperature (up to 12 Weeks)

### A. Materials and Equipment:

Forceps
Weigh pans
Glass lyo vials and caps
Stainless steel lyo tray
Paper grid for lyo tray
Chexall pouch, 12×18″
Foil pouch
PBS, 1×, without Ca and Mg
Trehalose—Cat #T0167, Sigma Aldrich
Epigallocatechin gallate (EGCG)—Cat #E4143, Sigma Aldrich
CellTiter-Glo Luminescent Cell Viability Assay kit
Lyophilizer (apparatus as described in Example 1 above)

### B. Procedure:

### Sample Preparation

1. Upon receiving processed milled cancellous bone tissue, weighed out a total of 10 samples of 0.5 g milled cancellous tissue granules each, into each of 10 glass lyo vials. (NOTE: cancellous tissue granules produced by the same process as described in Example 1 above, Part B.1.)
2. Weighed an additional 2 samples of 0.5 g each of untreated milled cancellous tissue for pre-contact (i.e., “Baseline”) cell viability testing.
3. Prepared lyopreservation solution as follows:
   a. Lyopreservation solution recipe:
      i. To the desired volume of HYCLONE® media, added EGCG at a concentration of 0.945 mg/mL and added trehalose at a concentration of 37.83 mg/mL to form lyopreservation solution.
      ii. Sterile filtered lyopreservation solution with a 0.2 um filter after preparation.
      iii. Example: For 78 mL of lyopreservation solution, added 0.0737 g of EGCG and 2.951 g of trehalose to 78 mL of HYCLONE® media.
4. Added 2 mL of lyopreservation solution to each of 10 vials containing the 10 weighed samples of milled cancellous bone tissue to form tissue-protectant mixtures.
5. Placed the 10 lyo vials (containing tissue-protectant mixtures) on stainless steel lyo tray and placed caps loosely on top of vials without sealing.
6. Carefully sealed lyo tray containing samples in a large autoclave pouch (paper side facing up). Ensured that vials and caps do not get knocked over and that the vials are not sealed.
7. Allowed cancellous tissue-protectant mixtures to incubate in lyopreservation solution at ambient temperature without agitation for a 3-3.5 hours.
8. After incubation was complete, lyopreserved all 10 processed tissue-protectant mixtures using 24 hour recipe provided in Table 4 below. Upon completion of the lyopreservation cycle, samples stoppered under vacuum prior to releasing lyo vacuum.

TABLE 4

| | Temperature (° C.) | Time (min) | Vacuum (mTorr) | Ramp/Hold |
|---|---|---|---|---|
| Thermal Treatment | | | | |
| Step 1: Freeze | −40 | 35 | — | Ramp |
| Freeze, Condenser, and | | | | |
| Vacuum Parameters | | | | |
| Freeze Temperature (° C.) | −40 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | 0 | 40 | 200 | Ramp |
| Step 2 | 0 | 360 | 200 | Hold |
| Step 3 | 15 | 15 | 200 | Ramp |
| Step 4 | 15 | 525 | 200 | Hold |
| Step 5 | 25 | 15 | 200 | Ramp |
| Step 6 | 25 | 360 | 200 | Hold |

9. Performed cell viability testing of 2 tissue samples set aside in step B.2 above, following procedures described above for the CellTiterGlo ATP Assay (test kit commercially available from Promega, located in Madison, WI, U.S.A), which provided two “Baseline” (pre-contact) ATP values which were averaged to provide a single “Average Baseline ATP Value” for use in calculating % of Baseline ATP and % retained populations of viable cells after 4, 8, 10 and 12 weeks of storage, as explained and reported below.
10. Sealed lyophilized samples in a Mangar foil pouch and stored at ambient conditions.

### C. Cell Viability Testing at 0, 4, 8, 10 and 12 Weeks

1. Immediately (i.e., about 1 hour, which is T0 or “Week 0”) after lyophilization and also at each of Weeks 4, 8, 10, and 12 of post-lyopreservation storage, removed 2 preserved tissue form samples for cell viability testing.
2. Prior to testing, rehydrated each of the preserved tissue form samples by adding 4 mL of PBS 1× to each vial and allowing to sit for 10 min at ambient conditions.
3. Cell viability testing was performed on each pair of samples pulled at each of Weeks 0, 4, 8, 10, and 12, following procedures described above for the CellTiterGlo ATP Assay (same as specified above). This viability testing provided two ATP values for each of Weeks 0, 4, 8, 10 and 12. The two ATP values obtained for Week 0 were averaged to provide an “Average Week 0 ATP Value,” and the same averaging calculation was performed for each of the Week 4, 8, 10 and 12 pairs of ATP values, to obtain Average Week X ATP Values. Results of cell viability testing are provided in Tables 5A and 5B below.
   In particular, Table 5A below provides % of Baseline (pre-contact) ATP for the two Week 0 samples of preserved cancellous bone tissue samples, which were calculated as follows:

$$\% of Baseline ATP = ( Week 0 ATP Value / Average Baseline ATP Value ) \times 100$$

TABLE 5A
Cell Viability at Post-Lyo (Week 0) Compared to Baseline

| Sample # | % of Baseline ATP |
|---|---|
| 1 | 38.5 |
| 2 | 35.8 |

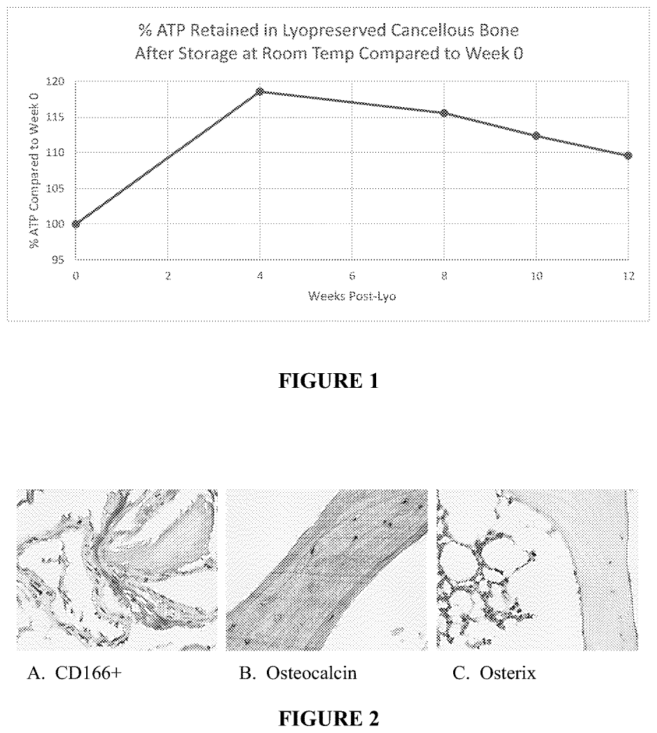

Table 5B below and FIG. 1 show the % Retained ATP at 4, 8, 10 and 12 weeks as compared to viability at Week 0, which is calculated as follows:

$$\% Retained ATP = ( Average Week X ATP Value / Average Week 0 ATP Value ) \times 100 ,$$

where X=4, 8, 10, 12, respectively.

TABLE 5B

| Timepoint | % Retained ATP* |
|---|---|
| Week 4 | 119% |
| Week 8 | 116% |
| Week 10 | 112% |
| Week 12 | 110% |

*Note:
Samples were not sealed under vacuum due to a mistake in setting up the samples in the lyophilizing apparatus. % Retained ATP in Table 5B greater than 100% is attributed to sample variability but otherwise proves stability of cell viability overtime.

### Example 3

Preserved Tissue Form Containing Both Viable Cancellous Bone Granules and Demineralized Cortical Bone Fibers with Demonstrated Viability After Lyopreservation and Storage at Room Temperature (up to 12 Weeks).

For this experiment, cancellous and cortical bone tissue samples from 3 different donors were obtained, processed and combined, as follows, to produce a total of 18 combined test samples. From each donor, cancellous tissue samples comprising cancellous bone were recovered and processed to provide cancellous granules, and cortical bone tissue samples were recovered and processed to provide cortical fibers. Quantities of cancellous granules and cortical fibers derived from the same donor were measured and combined to produce three combined test samples per donor, each of which contained about 40 wt % cancellous granules and about 60 wt % cortical fibers, the wt % being based on the total weight of the combined cancellous granules and cortical fibers of each sample. The total weights of the test samples (i.e., each comprising both cancellous granules and cortical fibers) ranged from 1.3 g to 1.6 g. The test samples were preserved after contact with protectants in solution, in accordance with the methods described hereinabove, and analyzed for cell viability by measuring ATP. The ATP results reported below are based on the mathematical average of the measured ATP results for each of the three test samples comprising both cancellous granules and cortical fibers and derived from a common donor.

### A. Protectant Solution Preparation:

1. Protectant solution was prepared by dissolving trehalose and EGCG into PBS 1× at concentrations of 0.4 M and 4 mM, respectively.
2. Following dissolution of preservative ingredients in media, the solution was sterile filtered using a 0.2 μm membrane filter.

### B. Sample Preparation (Performed in Sterile Hood)

3. Viable cancellous tissue (granules) and demineralized cortical tissue (fibers) were obtained by sterile processing techniques as described in Example 1 above, Part B.1.
4. Bulk cancellous granules and demineralized cortical fibers were packaged under sterile conditions and temporarily stored at refrigerated temperature (2-4° C.) for a period of time greater than 0 and up to 90 minutes, until time of use in experiment.
5. In a sterile hood, multiple samples were weighed out into 1 oz. jars, as follows:
   a. each sample included 0.5 g cancellous granules and 0.8 g cortical fibers.
6. 4 mL of sterile protectant solution was added to each jar containing tissue.
7. Tissue was allowed to incubate with protectant solution at room temperature for 1 hour.
8. After the incubation period, jars containing tissue were placed on an aluminum or stainless steel tray and placed in the Lyopreserving Apparatus.
9. Tissue was lyopreserved generally according to the recipe provided below in Table 6.

TABLE 6

| | Temperature (° C.) | Time (min) | Vacuum (mTorr) | Ramp/Hold |
|---|---|---|---|---|
| Thermal Treatment | | | | |
| Step 1: Freeze | −30 | 120 | — | Ramp |
| Freeze, Condenser, and | | | | |
| Vacuum Parameters | | | | |
| Freeze Temperature (° C.) | −30 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | −15 | 40 | 200 | Ramp |
| Step 2 | −15 | 1000 | 200 | Hold |
| Step 3 | 1 | 60 | 200 | Ramp |
| Step 4 | 1 | 600 | 200 | Hold |
| Secondary Drying Phase | | | | |
| Parameters | | | | |
| Secondary Set Point | 2 | | | |
| Post Heat Step | 2 | 5 | 100 | Hold |

Key: “Ramp” indicates variable temperature over the time period indicated (Time (min)).
“Hold” indicates temperature held constant over the time period indicated (Time (min))

### C. Sample Testing

10. Prior to contact with protectant solution (i.e., prior to step B.6. above), one or more test samples containing about 1.3-1.6 g of total tissue were measured and tested per the ATP viability assay procedures described above, which provided “Baseline” ATP values.
11. Prior to lyopreservation but post-contact and incubation with the protectant solution (i.e., prior to step B8 above), one or more test samples containing about 1.3-1.6 g of total tissue were measured and tested per the ATP viability assay procedures described above, which provided “Pre-Lyo” (i.e., “post-contact and incubation”) ATP values.
12. Following lyopreserving, samples were rehydrated by adding 1×PBS to each jar to the fill line and letting sit at room temperature for 5 minutes.
13. Following the 5 minute rehydration period, excess PBS solution was decanted from all jars.
14. For each sample, all the tissue (1.3-1.6 g) was tested per the ATP viability assay procedures described above and values corrected based on their pre-lyo weight, which provided Week 0 ATP values. The ATP data resulting from the foregoing experimental procedure was converted to % of Baseline ATP and % of Pre-Lyo ATP, as reported in Table 7A below, using the following formulas:

$$\% of Baseline ATP = ( Average Week 0 ATP value / Average Baseline ATP value ) \times 100$$

$$\% of Pre - Lyo ATP = ( Average Week 0 ATP value / Average Pre - Lyo ATP value ) \times 100$$

TABLE 7A

| | | % of | % of |
|---|---|---|---|
| | | Baseline | Pre-Lyo |
| Donor # | Description | ATP* | ATP |
| 1 | Cancellous granules with | 68% | 84% |
| | demineralized cortical fibers | | |
| 2 | Cancellous granules with | 36% | 48% |
| | demineralized cortical fibers | | |
| 3 | Cancellous granules with | 55% | 75% |
| | demineralized cortical fibers | | |

*Baseline samples = viable cancellous granules and demineralized bone fibers tested immediately prior to use in experiment, with no exposure to lyophilization solution, therefore, indicative of “pre-contact” population of viable cells

Table 7B below and shows the % Retained ATP 12 weeks as compared to viability at Week 0, which is calculated for each donor using the average ATP values for the 3 test sample replicates, as follows:

$$\% Retained ATP = ( Average Week 12 ATP Value / Average Week 0 ATP Value ) \times 100$$

where,

$$Average Week 0 ATP Value = [ Week 0 ATP Sample 1 + Week 0 ATP Sample 2 + Week 0 ATP Sample 3 ] / 3$$

$$Average Week 12 ATP Value = [ Week 0 ATP Sample 1 + Week 0 ATP Sample 2 + Week 0 ATP Sample 3 ] / 3$$

TABLE 7B

| | | % Retained ATP |
|---|---|---|
| Donor # | Description | (compared to 0 week)* |
| 1 | 12 Week Post-Lyo Cancellous | 110% |
| | granules with demineralized | |
| | cortical fibers | |
| 2 | 12 Week Post-Lyo Cancellous | 107% |
| | granules with demineralized | |
| | cortical fibers | |
| 3 | 12 Week Post-Lyo Cancellous | 95% |
| | granules with demineralized | |
| | cortical fibers | |

*% Retained ATP in Table 7B greater than 100% is attributed to sample variability but otherwise proves stability of cell viability overtime.

### Example 4

Comparison of Cryopreserved (DMSO) Viable Cartilage Fibers and Lyopreserved (trehalose/EGCG) Viable Cartilage Fibers

### A. Preparation of Cryopreservation and Lyoprotectant Solutions

1. Cryopreservation Solution-10% DMSO Hyclone
   a. Add 100 mL of sterile DMSO to 900 mL of sterile Hyclone media inside the biological safety cabinet.
   b. Invert 3-5 times to homogenize the solution.
2. Lyopreservative Solution-0.4 M Trehalose (151.32 mg/mL)/4 mM EGCG (1.9 mg/mL) in PBS
   a. Same preparation as in Example 1 (section A) above

### B. Viable Cartilage Fibers Preparation Procedure:

1. Distal Femur osteochondral (OC) grafts (tissue samples) were received and/or packaged in ambient storage media
   a. Composition of the ambient storage media was as follows:
      i. 0.95×DMEM,
      ii. supplemented with 0.125 uM Dexamethasone, and
      iii. 0.95× Antibiotic-Antimycotic (Penicillin/Streptomycin/Amphotericin B).
2. OC grafts in ambient storage media and necessary supplies and equipment were passed into a sterile hood (biological safety cabinet).
   a. All following steps are performed aseptically
3. One Nalgene container was filled with one full bag (250 mL) of ambient storage media.
4. An OC Graft was removed from its packaging and the leftover solution drained off, down the drain.
5. The OC Graft was placed in ACT Vise by clamping the bone portion of the graft into the vise by turning the handle until secure. Care was taken not to clamp or damage areas of cartilage.
6. Grating cartilage to produce viable cartilage fibers was performed by pressing firmly on the cartilage portion of tissue with an OXO® grater (Model #50581), using a stroking motion directed toward yourself. Using two hands facilitates the grating process. After four strokes, the grater was dipped in the Nalgene container with ambient storage media.
   a. Care was taken not to grate any areas of damaged cartilage or bone tissue
   b. The OC graft was repositioned in the ACT Vise as necessary to accomplish grating and formation of cartilage fibers
7. The time that the first portion of fibers are placed in the ambient storage media was recorded.
   a. Viable cartilage fibers must be collected in ambient storage media and drained within 3 hours of when the first set of fibers were soaked.
8. Step 5 was repeated until all cartilage was grated into fibers.
9. Once all cartilage was grated, the mesh basket designated was placed, with the lid off, over a sieve. The grated fibers and ambient storage media were poured into the basket. Once the storage media drained, the lid of the mesh basket was closed and secured. The basket was lifted up and down to drain residual storage media.
10. 1 liter of PBS was poured over the cartilage fibers in the basket to rinse off any excess media not removed during draining.
11. A Nalgene container was filled with PBS and the mesh basket placed into the container, ensuring that the fibers were submerged completely.
12. A 1 oz sample jar was tared on a scale.
13. The basket was lifted out of the container with PBS, bobbing it up and down and tilting back and forth until PBS no longer dripped from the basket.
14. The lid of the mesh basket was unsecured and opened.
15. Using forceps, the viable cartilage fibers were stirred and fluffed to release any surface tension from the PBS with the bottom of the mesh basket. This step is helpful to ensure that all the fibers are evenly wetted throughout the basket.
16. Using forceps, 0.3-0.4 g of the viable cartilage fibers were aliquoted into the 1 oz jar.
   a. Another quantity of fibers may be aliquoted. 0.3-0.4 g of fibers was sufficient for ATP testing performed as described herein.
17. 2 samples of viable cartilage fibers without any solution were set aside to be tested for baseline ATP (as reported in Table 8 below).
   a. Baseline samples were tested as described above (in Characterization Test Methods section) with CellTiter-Glo Reagent by Promega (Madison, WI) to obtain baseline cell viability.
18. Respective protectant media (Cryopreservation/Lyoprotectant Solution) were immediately poured into the jar for each cryo/lyo sample of cartilage fibers.
   a. For Cryopreservation Solution-solution was added to the fill line on the inside of the jar. The lid was tightly screwed shut.
   b. For Lyoprotectant Solution-solution was added until all fibers were completely submerged and lid screwed tightly shut.
19. For cryopreservation solution samples, the time the first jar was filled was recorded. All samples must be placed in the cryopreservation apparatus (Thermo Scientific CryoMed Model 7454, the “CryoMed”) and the CryoMed must be started within 2 hours from when the first sample jar was filled.
20. For lyoprotectant solution samples, the time the first jar was filled was recorded. All samples were incubated for at least one hour from when the first sample jar was filled (at ambient temperature, 19-23° C.).
   a. 3 samples were tested as described above (in Characterization Test Methods section) with CellTiter-Glo Reagent for pre-lyo cell viability after the 1-hour incubation was complete and after aliquoting of all other samples for cryopreservation was completed, representing a post-contact viability measure but prior to lyophilization.
21. Step 16 was repeated 5 times, and the mesh basket dipped back into the Nalgene container with PBS to re-wet the fibers in between. Again, steps 13-15 were performed to dry the fibers.
22. Fibers were aliquoted until all sample jars were filled or until all cartilage fibers produced from a particular OC graft were used up.

### C. Preservation Techniques

### Cryopreservation:

1. Each sample-containing jar to be cryopreserved was placed into a foil pouch horizontally and the pouches sealed.
2. The pouches were placed on the CryoMed rack and the rack was placed in the CryoMed when all samples were loaded.
3. The CryoMed was turned on and the desired cryopreservation recipe/procedure was selected, which is provided in Table 8 below:

TABLE 8

| | Target | Rate of Change | Time |
|---|---|---|---|
| Action | Temperature | (° C./minute) | (minutes) |
| Hold (Chamber) | 4° C. | — | 30 |
| Sample | 4° C. | −3 | — |
| temperature | | | |
| decrease \hat{ } | | | |
| Chamber | −90° C. | −10 | — |
| temperature | | | |
| decrease | | | |
| Hold (Chamber) | −90° C. | — | 20 |
| Hold (Chamber) | −85° C. | — | 25 |
| Sample temperature | −100° C. | −5 | — |
| decrease | | | |
| Hold (Chamber) | −100° C. | — | Until complete |

\hat{ } NOTE:
Rate of Change steps are all chamber temperature rates, with S and C being either Sample or Chamber target temperature

4. The CryoMed door was closed and the “Start” button pushed.
5. The CryoMed operating instructions to stop the cycle were performed after completion of the recipe.
6. With protective equipment; insulating gloves and face mask on, the rack was removed from the CryoMed and each foil pouch was opened and every sample-containing jar taken out of its foil pouch.
7. Samples were stored in a LN tank (−210 to −196° C.).
8. To prepare for testing for ATP, the samples were thawed (still in jars) in a water bath for 20-30 minutes.
9. Once completely thawed, for each sample, the cryopreservation solution was decanted from the jar and PBS added to the jar to the fill line to rinse cryopreservation solution from the fibers.
10. PBS was decanted from the jar to provide relatively dry fibers for cell viability testing with CellTiter Glo Reagent.
11. The cryopreserved samples were tested for cell viability as described above (in Characterization Test Methods section) with CellTiter-Glo Reagent.

### Lyophilization:

1. Each sample-containing jar to be lyopreserved was placed onto an aluminum or stainless steel dish and inserted into the lyophilizer (apparatus as described in Example 1 above).
2. The lyophilizer was turned on and the desired lyopreserving recipe/procedure was selected, which is provided in Table 9 below:

TABLE 9

| | Temperature (° C.) | Time (min) | Vacuum (mTorr) | Ramp/Hold |
|---|---|---|---|---|
| Thermal Treatment | | | | |
| Step 1: Freeze | −30 | 120 | — | Ramp |
| Freeze, Condenser, and | | | | |
| Vacuum Parameters | | | | |
| Freeze Temperature (° C.) | −30 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | −15 | 40 | 200 | Ramp |
| Step 2 | −15 | 1000 | 200 | Hold |
| Step 3 | 1 | 60 | 200 | Ramp |
| Step 4 | 1 | 600 | 200 | Hold |
| Secondary Drying Phase | | | | |
| Parameters | | | | |
| Secondary Set Point | 2 | | | |
| Post Heat Step | 2 | 5 | 100 | Hold |

3. When the recipe was complete, the sample-containing jars were removed from the lyophilizer and packaged in Mangar foil pouches to prevent moisture absorption.
4. In preparation for cell viability testing, the lyopreserved viable cartilage fibers were rehydrated with PBS 1× by filling each 1 oz jar to the fill line.
5. Allowed tissue to sit for 2 minutes before decanting excess PBS 1× using gauze.
6. The lyopreserved samples were tested for cell viability as described above (in Characterization Test Methods section), and specifically as described below, using CellTiter-Glo reagent.

### D. Cell Viability Testing-Cell Titer Glo Viability Assay:

1. The CellTiter-Glo reagent was thawed in a water bath at room temperature. At least 20 minutes was allowed for a full 100 mL set to thaw.
2. Once thawed, the bottle was inverted multiple times and rolled to homogenize the reagent. Reagent was stored away from light until use.
3. DMEM was aliquoted into a 50 mL tube and allowed to reach room temperature.
4. 0.3-0.4 g of viable cartilage fibers were placed into a 15 mL tube.
5. 2000 uL of DMEM was added into each tube with cartilage fibers.
6. 2000 uL of CellTiter-Glo was added into each tube with cartilage fibers.
7. A blank was created by mixing 1000 uL DMEM and 1000 uL CellTiter-Glo into a 15 mL that does not contain any cartilage fibers.
8. All tubes were inverted 5 times in quick succession and placed in a rack.
9. Protected from light, the tubes were placed on an orbital shaker and shaken for 5 minutes at 150 rpm at room temperature.
10. The tubes were then placed on the bench top for 20 minutes, still protected from light.
11. After the 20 minute incubation, the tubes were centrifuges tubes at 3000 rpm for 3 minutes.
12. The solution from each tube was distributed into an opaque 96-well plate.
   a. 200 uL from each tube was transferred to 6 wells of the 96-well plate.
   b. 200 uL of the blank was plated in quadruplicate.
13. The plate was covered with aluminum foil to protect from light and placed and shaken on an orbital shaker for 2 minutes at 150 rpm.
14. After 2 minutes, the luminescence of the plate referencing WI-330 was read and recorded.

### E. Cell-Viability Results:

This experiment sought to establish whether lyophilization of viable cartilage fibers could preserve cell viability. As described above, viable cartilage fibers were produced by grating from osteochondral grafts stored in ambient storage media. Post-grating, viable cartilage fibers were subsequently incubated in lyoprotectant media containing protectants, i.e., trehalose and EGCG, for one hour and lyopreserved. Viability was assessed via ATP assay whereby CellTiter-Glo by Promega (Madison, WI) was used to measure ATP content. Results are shown below in Tables 10A & 10B.

TABLE 10A
Week 0 Post-Lyo & Post-Cryo Cartilage Fiber Cell Viability Results

| Sample | % of Baseline ATP | % of Pre-Lyo ATP |
|---|---|---|
| T0 Post-Lyopreservation | 4% | 16% |
| T0 Post-Cryopreservation | 10% | 36% |

TABLE 10B
% Retained ATP for 1 year timepoint Post-Lyo & Post-Cryo
Cartilage Fiber compared to week 0

| | % Retained ATP |
|---|---|
| Sample | (compared to 0 week)* |
| Post-Lyo 1 Year | 54% |
| Post-Cryo 1 Year | 142% |

*% Retained ATP in Table 10B greater than 100% is attributed to sample variability but otherwise proves stability of cell viability overtime.

### Example 5

### Comparison of Cryopreserved (DMSO) Viable Cancellous Bone Granules and Lyopreserved (Trehalose/EGCG) Viable Cancellous Bone Granules

### A. Preparation of Cryopreservation and Lyoprotectant Solutions

1. Cryopreservation Solution—10% DMSO Hyclone
   a. Add 100 mL of sterile DMSO to 900 mL of sterile Hyclone media inside the biological safety cabinet.
   b. Invert 3-5 times to homogenize the solution.
2. Lyopreservative Solution—0.1 M Trehalose (37.83 mg/mL)/2 mM EGCG (0.945/mL) in Hyclone media.
   a. Same preparation as in Example 1 (section A) above

### B. Procedure (Performed in Sterile Hood):

### Sample Preparation—Lyophilization

1. Upon receiving milled cancellous bone tissue, about 0.5 g of milled cancellous tissue granules per sample were weighed and placed into 1 oz jars. (NOTE: cancellous tissue granules produced by the same process as described in Example 1 above, Part B.1.)
2. Two additional samples of 0.5 g milled cancellous tissue were weighed and set aside for Baseline cell viability testing.
3. Added 4 mL of lyopreservation solution to each jar containing weighed samples of milled cancellous bone tissue designated for lyophilization to form tissue-protectant mixtures.
4. Placed lyo jar (containing tissue-protectant mixtures) on stainless steel lyo tray and tightly sealed a vented cap with porous liner on the jar.
5. Carefully sealed lyo tray containing samples in a large autoclave pouch (paper side facing up)
6. Allowed cancellous tissue-protectant mixtures to incubate in lyopreservation solution at ambient temperature without agitation for 1 hour.
7. After incubation was complete, lyopreserved all processed tissue-protectant mixtures using 24 hour recipe provided in Table 11 below. Upon completion of the lyopreservation cycle, samples stoppered under vacuum prior to releasing lyo vacuum.

TABLE 11
Lyophilization Recipe for Example 5

| | Temperature (° C.) | Time (min) | Vacuum (mTorr) | Ramp/Hold |
|---|---|---|---|---|
| Thermal Treatment | | | | |
| Step 1: Freeze | −30 | 120 | | Ramp |
| Freeze, Condenser, and | | | | |
| Vacuum Parameters | | | | |
| Freeze Temperature (° C.) | −30 | | | Hold |
| Additional Freeze (min) | | 180 | | |
| Condenser Set Point (° C.) | −60 | | | |
| Vacuum Set Point (mTorr) | | | 500 | |
| Drying Phase Parameters | | | | |
| Step 1 | 0 | 40 | 200 | Ramp |
| Step 2 | 0 | 360 | 200 | Hold |
| Step 3 | 15 | 15 | 200 | Ramp |
| Step 4 | 15 | 525 | 200 | Hold |
| Step 5 | 25 | 15 | 200 | Ramp |
| Step 6 | 25 | 360 | 200 | Hold |

### Sample Preparation—Cryopreservation

1. Upon receiving milled cancellous bone tissue, 0.5 g of milled cancellous tissue per sample were weighed and placed into 1 oz jars.
2. The same baseline samples as the lyopreservation sample preparation step were utilized whereby 0.5 g of milled cancellous tissue were tested for baseline cell viability.
3. For cryopreservation samples, cryopreservation solution was added to the fill line on the inside of the jar. The lid was tightly screwed shut.
4. The same cryopreservation technique as in Example 3 (Section C) was used.

### C. Cell Viability Testing Post-Lyo and Post-Cryo

1. Cryopreservation samples were set out on a benchtop and thawed at room temperature
2. Once thawed, cryopreservation samples were decanted of cryoprotectant solution, rinsed with PBS filling the jar up to the fill-in, and once again decanted.
3. At the same time, lyopreservation samples were rehydrated by adding 1×PBS to each jar until the fill line and allowed to rest for 5 minutes at ambient conditions.
4. After 5 minutes, excess solution was decanted from the lyopreservation samples.
5. Cell viability testing was performed following procedures described above for the CellTiterGlo ATP Assay (same as specified above).
6. For each sample, decanted tissue (about 0.48 g) was weighed out and tested per the ATP viability assay procedures described above, which provided Week 0 ATP values. The ATP data resulting from the foregoing experimental procedure was converted to % of Baseline ATP, as reported in Table 12 below, using the following formula:

$$\% of Baseline ATP = ( Average Week 0 ATP value / Average Baseline ATP value ) \times 100$$

TABLE 12

| Sample | | Average % of |
|---|---|---|
| Set # | Description | Baseline ATP* |
| 1 | Two replicates of lyopreserved viable cancellous granules | 41.6% |
| 2 | Two replicates of cryopreserved viable cancellous granules | 46.2% |

### Example 6

### Lyopreserved Viable Amnion/Chorion Bilayer Tissue Forms

A. Preparation of Lyopreserved Amnion/Chorion Bilayer with Trophoblast Layer
1. A solution of 100 IU/mL Streptokinase in red blood cell lysis buffer was prepared (SK-RBC lysis buffer solution).
2. Lyoprotectant Solution was prepared as described in Example 1 above but having the following content: 0.5 M trehalose, 4 mM Epigallocatechin gallate (EGCG), in HBSS
   2.1. The lyoprotectant solution was protected from light and refrigerated until time of use.
3. The amnion membrane was peeled apart from the chorion membrane by hand and then cut free from the umbilical cord with scissors.
4. The chorion membrane was cut free from the placental disk with scissors.
5. Large blood clots on the chorion were gently dabbed using wetted wipes and the chorion was placed into a flask of 500 ml SK-RBC lysis buffer solution.
6. The flask was placed onto an orbital shaker and agitated at 150 RPM for 90 minutes.
7. Meanwhile, the amnion was manually cleaned, by dabbing and wiping with wetted wipes while soaking in HBSS to remove blood and blood clots, two times for 10-20 minutes each time.
   7.1. After cleaning, the amnion was left in the second HBSS solution while waiting for chorion agitation to finish.
8. After agitation in the SK-RBC lysis buffer solution, the chorion was removed from the flask and transferred into a container of HBSS.
9. The chorion was manually cleaned by dabbing and wiping with wetted wipes and cotton applicators while soaking in the HBSS to remove residual blood and blood clots.
10. Antibiotic solution was prepared by making a solution of 50 ug/mL vancomycin, 50 ug/mL gentamycin, and 2.5 ug/mL amphotericin B in HBSS.
11. The amnion and chorion were transferred into a flask with antibiotic solution and agitated at 65 RPM for 60 minutes.
12. The amnion and chorion were transferred into a flask with fresh HBSS and agitated at 65 RPM for 5 minutes.
13. The amnion and chorion were transferred into a flask with fresh HBSS and agitated at 65 RPM for 5 minutes.
14. The amnion and chorion were transferred into a flask with fresh HBSS with phenol and agitated at 65 RPM for 30 minutes, to impart a reddish color to the tissue for easier visualization during cutting.
15. The amnion was transferred into a basin of fresh HBSS with phenol red to keep the reddish color.
16. The chorion was transferred into a basin of fresh HBSS and then manually cleaned by dabbing and wiping with wetted wipes and cotton applicators while soaking in HBSS to remove residual blood and blood clots.
17. The amnion was placed on a backing material with epithelial side facing down, toward the backing.
18. The chorion was placed on top of the amnion, with the trophoblast side facing upward, away from the amnion.
19. The layered amnion/chorion composite was cut using a rolling bladed cutter into samples of approximately 5×5 cm.
20. The amnion/chorion sample still on backing was placed into a snap retainer and then immersed into HBSS until all cutting was completed.
   20.1. Several tissue containment configurations were tested across donors, such as placing tissue on backing into a netting mesh, placing tissue on netting mesh into a retainer, and placing tissue on backing into a retainer.
21. The retainers containing amnion/chorion tissue were placed into lyoprotectant solution and refrigerated for 30-60 minutes.
22. After 30-60 minutes, the retainers were removed from solution and transferred into regular HBSS for 5 minutes.
23. Each retainer was drained of excess liquid by dabbing on its side against sterile wipes and then packaged and sealed into a breathable Tyvek pouch.
24. The sealed pouches were placed with breathable side up into a lyophilizer (apparatus as described in Example 1 above) and lyophilized using a programmed recipe as described in Tables 13-17 provided below.
25. After lyo cycle was completed, pouches were removed and sealed into foil pouches as a moisture barrier pouch.
   B. Amnion/Chorion Bilayer without Trophoblast Layer
1. Placenta Tissue Sample was processed as previously described above in Example 6, Part A, until Step 16, when chorion was being cleaned after antibiotic soak.
2. Before spot cleaning residual blood and blood clots, the trophoblast layer of the chorion was removed by gently peeling loose the trophoblast layer using fingers, cotton applicators, instruments such as dissecting forceps, or wetted wipes.
   2.1. The removal of the trophoblast layer also resulted in the removal of residual blood and blood clots embedded within the tissue, leaving very little residual blood on the remaining chorion.
3. Further processing with the amnion and thinner chorion was performed as previously described above in Example 6, Part A, Step 17 and onward.
   The following Table 13 provides a summary of the features and conditions of each of the above-described Bilayer Lyopreserved Tissue Forms, both with and without trophoblast layer, including the lyopreservation recipe performed in the lyophilizer, the packaging configuration and the tissue configuration. The Iteration #corresponds to placental tissue samples recovered from different donors, or to different lyo recipes or packaging configurations within the same donor.

TABLE 13

| Iteration | Lyo | | Tissue |
|---|---|---|---|
| # | Recipe | Packaging Configuration | Configuration |
| 1 | RTT | On backing, in netting | AM and CM separate |
| 2 | RTAC v1 | between 2 netting, in retainer | AM/CM composite |
| 3 | RTAC v2 | between 2 netting, in retainer | AM/CM composite |
| 4 | RTAC v2 | between 2 backing, in retainer | AM/CM composite |
| 5 | RTAC v2 | on backing, in retainer | AM/CM composite |
| 6 | RTAC v2 | on backing, in retainer | AM/CM composite |
| 7 | RTAC v3 | on backing, in retainer | AM/CM composite |
| 8 | RTAC v3 | on backing, in retainer | AM/CM composite |
| 9 | RTAC v3 | on backing, in retainer | AM/CM composite |
| 10 | RTAC v3 | on backing, in retainer | AM/CM composite |
| 11 | RTAC v3 | on backing, in retainer | AM/CM composite |

The lyopreservation recipes identified in Table 13 above (i.e., RTT, RTAC v1, RTAC v2, and RTAC v3) as performed on the above-described Bilayer Lyopreserved Tissue Forms are provided in Tables 14, 15, 16, and 17 below.

TABLE 14
RTT Recipe

| | Temperature | Time | Vacuum | Ramp/ |
|---|---|---|---|---|
| Recipe Phase | (° C.) | (min) | (mTorr) | Hold |
| Thermal Treatment | | | | |
| Step 1 | −30 | 120 | | Ramp |
| Freeze Temperature | −30 | | | Hold |
| Additional Freeze Time | | 180 | | |
| Condenser Set Point | −60 | | | |
| Vacuum Set Point | | | 500 | |
| Drying Phase | | | | |
| Step 1 | −15 | 40 | 200 | Ramp |
| Step 2 | −15 | 1000 | 200 | Hold |
| Step 3 | 1 | 60 | 200 | Ramp |
| Step 4 | 1 | 600 | 200 | Hold |
| Secondary Drying Phase | | | | |
| Post Heat Step | 2 | 5 | 100 | Hold |

TABLE 15
RTAC v1

| | Temperature | Time | Vacuum | Ramp/ |
|---|---|---|---|---|
| Recipe Phase | (° C.) | (min) | (mTorr) | Hold |
| Thermal Treatment | | | | |
| Step 1 | −40 | 60 | | Ramp |
| Freeze Temperature | −40 | | | Hold |
| Additional Freeze Time | | 30 | | |
| Condenser Set Point | −40 | | | |
| Vacuum Set Point | | | 600 | |
| Drying Phase | | | | |
| Step 1 | −10 | 720 | 600 | Hold |
| Secondary Drying Phase | | | | |
| Post Heat Step | 25 | 240 | 600 | Hold |

TABLE 16
RTAC v2

| | Temperature | Time | Vacuum | Ramp/ |
|---|---|---|---|---|
| Recipe Phase | (° C.) | (min) | (mTorr) | Hold |
| Thermal Treatment | | | | |
| Step 1 | −40 | 120 | | Ramp |
| Freeze Temperature | −40 | | | Hold |
| Additional Freeze Time | | 30 | | |
| Condenser Set Point | −40 | | | |
| Vacuum Set Point | | | 600 | |
| Drying Phase | | | | |
| Step 1 | −10 | 720 | 600 | Hold |
| Secondary Drying Phase | | | | |
| Post Heat Step | 25 | 240 | 600 | Hold |

TABLE 17
RTAC

| | Temperature | Time | Vacuum | Ramp/ |
|---|---|---|---|---|
| Recipe Phase | (° C.) | (min) | (mTorr) | Hold |
| Thermal Treatment | | | | |
| Step 1 | 20 | 15 | — | Hold |
| Step 2 | −20 | 160 | — | Ramp |
| Step 3 | −40 | 60 | — | Hold |
| Freeze Temperature | −40 | | | Hold |
| Additional Freeze Time | | 30 | | |
| Condenser Set Point | −40 | | | |
| Vacuum Set Point | | | 600 | |
| Drying Phase | | | | |
| Step 1 | −5 | 520 | 600 | Hold |
| Secondary Drying Phase | | | | |
| Post Heat Step | 25 | 300 | 600 | Hold |

### C. Cell Viability Analysis via ATP Assay

A unit of lyopreserved viable amnion/chorion bilayer tissue form (prepared as described above in Example 6, Parts A-B) was cut into approximately 4 cm² pieces for the ATP assay. Each piece was rehydrated by immersing in PBS for 10 minutes, and the amnion and chorion layers were peeled apart using dissecting forceps to test the layers by ATP assay separately. Each approximately 4 cm² rehydrated tissue piece was combined with 0.5 mL DMEM and 0.5 mL assay reagent in a well plate and incubated on an orbital shaker in the dark for 20 minutes at room temperature. After incubation, aliquots of the incubated reagent were transferred into a 96-well plate and raw luminescence values (in RLU) were obtained using a plate reader. A standard curve was prepared by creating serial dilutions of known concentrations of an ATP standard, combining with assay reagent, incubating on an orbital shaker, and then reading the RLU of each dilution. The dilutions' RLUs were plotted against the known concentrations of each serial dilution to generate an equation, which was then used to convert RLU of the tissue pieces to concentrations of ATP. Finally, the tested tissue pieces were rinsed in water, placed into pre-weighed weighing pans, and dried in a gravity oven. The dried tissue and pans were then weighed to obtain dry tissue weights, and the concentration of ATP per sample was converted to amount of ATP per dry weight, and the amounts of ATP converted to % Pre-Lyo ATP retained in the Lyopreserved Amnion/Chorion Bilayer Tissue Form with Trophoblast Layer (Example 6, Part A), are reported in Table 18, using the following formula:

$$\% of Pre - Lyo ATP = ( Week 0 ATP value / Pre - Lyo ATP value ) \times 100$$

TABLE 18

| Iteration # | % of Pre-Lyo ATP, AM+ | % of Pre-Lyo ATP, CM+ |
|---|---|---|
| 1 | 3.90% | 1.46% |
| 2 | 7.47% | 1.87% |
| 3 | 7.72% | 1.67% |
| 4 | 27.06% | 1.52% |
| 5 | 25.04% | 2.10% |

* “AM+” and “CM+” refer to “Amnion” and “Chorion” with trophoblast layer, lyophilized as a bilayer, rehydrated for 10 min in PBS then peeled apart prior to testing ATP separately

Similarly, the amounts of ATP converted to % Pre-Lyo ATP retained in the Lyopreserved Amnion/Chorion Bilayer Tissue Form without Trophoblast Layer (Ex. 4B), are reported in Table 19 below.

TABLE 19

| Iteration # | % of Pre-Lyo ATP, AM− | % of Pre-Lyo ATP, CM− |
|---|---|---|
| 1 | N/A | 13.12% |
| 2 | 5.92% | 1.82% |
| 3 | N/A | 2.61% |

* “AM−” and “CM−” refer to “Amnion” and “Chorion” without trophoblast layer, lyophilized as a bilayer, rehydrated for 10 min in PBS then peeled apart prior to testing ATP separately
\hat{ } AM/CM-could not be separated for testing for Iteration #3

### Example 7

### Cell Viability Evaluation via Enzymatic Digestion and Live/Dead Fluorescent Staining

### List of Materials and Equipment Used

1. Collagenase Type II 1 gram (Worthington Biochemicals, Cat. LS004196)
2. 0.25% Trypsin EDTA 1× (Mediatech, Cat. 25-053-CI)
3. Phosphate Buffered Saline 1× (Mediatech, Cat. 21-040-CM)
4. Dulbecco's Modification of Eagle's Medium/Ham's F-12 50/50 Mix without phenol red (Mediatech, Cat. 16-405-CV)
5. Dulbecco's Phosphate-Buffered Saline, 1× (Mediatech, Cat. 21-031-CM)
6. Penicillin-Streptomycin Solution, 100× (Mediatech, Cat. 30-002-CI)
7. Fetal Bovine Serum, Premium (Heat Inactivated) (Mediatech, Cat. 35-016-CV or equivalent)
8. Corning glutaGRO, Liquid (Mediatech, Cat. 25-015-CI)
9. LIVE/DEAD Viability/Cytotoxicity Kit (Invitrogen, Cat. L3224)
10. CO₂ Incubator (NuAire)
11. 6-well; Standard tissue culture; Flat-bottom (Fisher Scientific, Cat. 08-772-1B)
12. 100 μm cell strainer (Fisher Scientific, Cat. 22-363-549)

### Tissue Processing:

1. Amnion/Chorion Bilayer with Trophoblast (AM+/CM+) and Amnion/Chorion Bilayer without Trophoblast (AM−/CM−) 5 cm×5 cm samples were prepared as described above in Example 6, but Lyoprotectant Solution was made with 0.3 M Trehalose and no EGCG. One sample of each tissue configuration was lyopreserved using RTACv2 lyo recipe (Iteration #6) and one sample of each tissue configuration was lyopreserved using RTACv3 lyo recipe (Iteration #7).

### Tissue Rehydration and Equilibration:

1. Supplemented DMEM/F12 media was prepared by adding 10% FBS, 1% PenStrep solution, and 1% glutaGRO solution final v/v to DMEM/F12 media without phenol red (i.e. 10 mL FBS, 1 mL PenStrep solution, 1 mL glutaGRO solution to 88 mL of DMEM/F12). The prepared media was stored at 4° C. in the dark. Immediately before use, the prepared media was warmed in a 37° C. water bath.
2. 12 days after completion of lyo recipe, each lyopreserved 5 cm×5 cm amnion/chorion bilayer sheet sample was removed from the outer foil pouch, inner Tyvek pouch, retainer, and backing, and immersed in a weigh pan of room temperature PBS for approximately 10 minutes. After rehydration, the amnion and chorion membranes were peeled apart and each membrane from each sample was placed into a separate well of a 6 well tissue culture plate, to which 5 mL of supplemented DMEM/F12 culture media was added. Each 5 cm×5 cm amnion or chorion membrane was allowed to equilibrate in media overnight in a CO₂ incubator at 37° C. and 5% CO₂ level.

### Enzymatic Tissue Digestion:

1. A 0.75% collagenase II solution was prepared by adding 1 g of powdered collagenase II per 133 mL of DMEM/F12 media without phenol red. The collagenase II solution and 0.25% trypsin solution were each warmed in a 37° C. water bath immediately prior to their use in the subsequent steps.
2. Each 5 cm×5 cm amnion (AM+, AM−) and chorion sample (CM+, CM−) was placed into a separate 50 ml conical tube containing 40 mL of collagenase II solution. Each conical tube was capped and laid sideways on an incubator shaker at 65 rpm for 40 minutes at 37° C., and then centrifuged at 2000 rpm for 10 minutes at ambient temperature. Taking care not to aspirate tissue, liquid in each tube was aspirated down to 5 mL remaining, and then 40 mL of 0.25% trypsin was added. Each conical tube was recapped and laid sideways on an incubator shaker at 65 rpm for 15 minutes at 37° C. After shaking, 5 mL of Fetal Bovine Serum was added to each tube to neutralize the trypsin.
3. Each tube's contents was poured through a separate 100 μm cell strainer and into a new 50 ml conical tube, to remove remaining tissue and debris. The new conical tubes were centrifuged at 2000 rpm for 10 minutes at ambient temperature. The liquid in each tube was aspirated down to 5 mL remaining, and then the last 5 mL of solution was transferred into a 15 mL conical tube. The 15 mL conical tubes were centrifuged at 2000 rpm for 10 minutes at ambient temperature. The supernatant from each conical tube was aspirated out of each tube and 1 mL of DMEM/F12 without phenol red was added to each. The contents of each tube was pipetted up and down for a minimum of 10 times to resuspend the cells, until no visible cell pellets were visible.
4. The cell suspension was transferred into a separate microcentrifuge tube for performing live/dead staining, as described below.

### Live/Dead Staining and Cell Count:

1. Because the live/dead stain is light sensitive, all live/dead staining steps were performed in the dark. The reagents from a live/dead viability kit (LIVE/DEAD® Viability/Cytotoxicity Kit, Invitrogen) were thawed at room temperature. 20 μL of 2 mM ethidium homodimer-1 (EthD-1) was added to 10 mL Dulbecco's Phosphate-Buffered Saline (DPBS) to form an EthD-1 solution, which was vortexed to mix. 5 μL of 4 mM calcein AM was added to the EthD-1 solution and vortexed to mix. The live/dead working solution (4 μM EthD-1, 2 μL calcein AM) was kept in foil to avoid exposing the solution to light before it was applied to the samples of enzymatically digested cell suspension, as follows.
2. Each microcentrifuge tube containing enzymatically digested cell suspension was centrifuged in a microcentrifuge for 6 minutes at 300×g. The resulting supernatant was removed by aspirating. 1 mL of the live/dead working solution was added to each microcentrifuge tube, followed by pipetting up and down to resuspend cells. The microcentrifuge tubes were incubated in the dark for 20 minutes at room temperature.
3. The tubes were centrifuged for 6 minutes at 300×g, supernatant was removed from each tube by aspirating, followed by adding 1 mL of fresh DPBS to each microcentrifuge tube and pipetting up and down to resuspend cells again. The foregoing centrifuge-aspirate-pipette cycle was repeated twice more, for a total of three cycles. 50 μL of each cell suspension was pipetted onto a separate microscope slide and carefully covered with a microscope coverslip, avoiding creating air bubbles.
4. Because the live/dead stain is light sensitive, the following live/dead cell enumeration steps to produce a representative image for each sample were also performed in the dark. Cells on each slide were visualized using a dual FITC/TRITC filter on the microscope, at 100% light power and using a 0.14-second manual exposure. EthD-1 (indicating dead cells) was red and calcein AM (indicating live cells) was green. Each coverslip was divided approximately evenly into multiple smaller sampling areas (as shown below) and a representative image taken from each area.

| | | |
|---|---|---|
| 1 | 2 | 3 |
| 4 | 5 | 6 |
| 7 | 8 | 9 |

5. A public domain, Java-based image processing program known as IMAGEJ Version 1.51j8 was used to analyze each representative image produced by the foregoing steps and determine the number of viable cells and nonviable cells. IMAGEJ was obtained from the National Institutes of Health, of Bethesda, Maryland, USA. The software was downloaded from the NIH website at https://imagej.nih.gov/ij/.
6. Using IMAGEJ, for each representative image, the image was opened and a conversion scale applied by drawing a line over the scale bar and going to Analyze→Set Scale. The known distance and units were entered. The “Global” option was checked to set a global scale, which applied a size limit for particle analysis at a later step. Menu options Image→Adjust→Threshold were successively selected. A hue filter (50-255 pass) and a brightness filter (150-255 pass) with B&W threshold color to filter out low intensity and/or red stains were selected/applied. Menu options Analyze→Analyze Particles were successfully selected. A size limit of 30-800 um² was applied to filter out small artifacts and larger debris and then the “Summarize” option was checked.
7. For each representative image processed as described above using IMAGEJ, the number of viable cells as indicated by a relatively strong green fluorescence with a stain size appropriate for a cell were reported. The nonviable cells were either manually counted, or the foregoing procedure was repeated for red stains (i.e., 0-50 pass instead of 50-255 pass hue filter, 100-255 pass brightness filter, and 10-300 um\hat{ }2 particle size). However, whenever possible, nonviable cells were manually counted because automated counts by IMAGEJ were known to sometimes return false positives because EthD-1 stains nuclei, not whole cells, and the stained nuclei are closer in size to non-cellular artifacts. The % Viable Cells for each representative image was calculated as follows, which provides a snapshot look at cell viability in each sample at the time of enzymatic digestion:

$$\frac{Viable Cells}{Viable cells + Nonviable Cells}$$

The two iterations, both with (Table 20) and without trophoblast layer (Table 21) included, showed that a high % cell viability could be obtained in the lyopreserved amnion/chorion bilayer after 12 days in storage.

TABLE 20

| Iteration | | |
|---|---|---|
| # | % Viable Cells, AM+ | % Viable Cells, CM+ |
| 6 | 91.59% | 88.39% |
| 7 | 92.78% | 93.06% |

* “AM+” and “CM+” refer to “Amnion” and “Chorion” with trophoblast layer, lyophilized as a bilayer, rehydrated for 10 min in PBS then peeled apart prior to testing as described

TABLE 21

| Iteration | | |
|---|---|---|
| # | % Viable Cells, AM− | % Viable Cells, CM− |
| 6 | N/A | 89.65% |
| 7 | 95.84% | 96.38% |

* “AM−” and “CM−” refer to “Amnion” and “Chorion” without trophoblast layer, lyophilized as a bilayer, rehydrated for 10 min in PBS then peeled apart prior to testing as described
** insufficient cells were recovered to perform counting of AM− digested cells for Iteration #6

### Example 8

Cell Viability Testing after Storage, at 0, 1, 2, 3 and 4 Weeks
1. Amnion/Chorion Bilayer with Trophoblast (AM+/CM+) samples were processed and preserved as previously described in Example 6, but with Lyoprotectant Solution containing 0.3 M trehalose with either 0 mM EGCG or 2 mM EGCG, lyopreserved using RTAC v3 lyo recipe, and then sealed into individual foil pouches. Immediately (i.e., about 1 hour) after lyophilization (referred to as “0 week”), or at 1, 2, 3 and 4 week timepoints, removed 1 preserved AM/CM+ sample per EGCG condition for cell viability testing as described in Cell Viability Analysis via ATP Assay above in Example 6. Results of cell viability testing are provided in Tables 22 and 23 below, as a % of Week 0 ATP values. Several samples of CM+ reported average ATP values above 100%, most likely due to the highly variable nature of the metabolic activity of the placental tissue as well as variability in the trophoblast tissue layer thickness for each sample, which would impact the average cell density per tissue volume and thus the normalized nnmol of ATP/g of tissue.

TABLE 22
0 mM EGCG

| | Iteration #8 | | Iteration #9 | | Iteration #10 | |
|---|---|---|---|---|---|---|
| | AM+ | CM+ | AM+ | CM+ | AM+ | CM+ |
| 0 weeks | 100.00% | 100.00% | 100.00% | 100.00% | 100.00% | 100.00% |
| 1 week | 50.87% | 106.62% | 62.04% | 125.53% | 104.13% | 84.23% |
| 2 weeks | 57.84% | 163.10% | 93.47% | 120.54% | 84.49% | 64.73% |
| 3 weeks | 54.12% | 293.83% | 65.17% | 126.18% | 123.91% | 63.92% |
| 4 weeks | 71.66% | 382.72% | 102.29% | 125.90% | 108.88% | 26.74% |

TABLE 23
2 mM EGCG

| | Iteration #8 | | Iteration #9 | | Iteration #10 | |
|---|---|---|---|---|---|---|
| | AM+ | CM+ | AM+ | CM+ | AM+ | CM+ |
| 0 weeks | 100.00% | 100.00% | 100.00% | 100.00% | 100.00% | 100.00% |
| 1 week | 46.79% | 86.47% | 67.53% | 68.63% | 76.34% | 86.98% |
| 2 weeks | 50.31% | 129.91% | 76.89% | 70.46% | 62.91% | 50.39% |
| 3 weeks | 39.60% | 138.04% | 60.79% | 77.26% | 86.63% | 60.35% |
| 4 weeks | 41.94% | 53.59% | 35.65% | 41.84% | 36.62% | 27.74% |

### Example 9

Comparison of Cell Viability Testing Post-Lyo vs. Post-Cryo
1. Amnion/Chorion Bilayer with Trophoblast (AM+/CM+) samples were processed and either lyopreserved as previously described in Example 6 but with Lyoprotectant Solution containing 0.3 M trehalose and no EGCG, or cryopreserved as described below. Additionally, Amnion single layer and Chorion single layer with Trophoblast samples were also processed and either lyopreserved in the same manner as the lyopreserved bilayer samples, or cryopreserved in the same manner as the cryopreserved bilayer samples.

### Cryopreservation:

1. Tissue was processed as described in Example 6 up through Step 20 (cutting 5 cm×5 cm samples and placing each into a retainer in HBSS until all cutting was complete).
2. After all samples were cut, the samples in retainers designated for cryopreservation were each placed into a film pouch. Each film pouch was filled with approximately 22.5 mL of cryopreservation media (Hyclone media with 10% DMSO), sealed, and then placed into an outer pouch. The samples and a representative probe sample were then cryopreserved using a controlled-rate freezer (Thermo Scientific CryoMed Model 7454) per the recipe provided in Table 24 below and stored then at −70° C. or colder.

TABLE 24
CryoMed recipe

| Step # | Parameter |
|---|---|
| 1 | Cool chamber 2° C./min until Sample reaches −4° C. |
| 2 | Cool chamber 25° C./min until Chamber reaches −60° C. |
| 3 | Heat chamber 10° C./min until Chamber reaches −12° C. |
| 4 | Cool chamber 1.5° C./min until Chamber reaches −40° C. |
| 5 | Cool chamber 2° C./min until Sample reaches −100° C. |
| 6 | Hold chamber at −100° C. |
| 7 | End |

### Cell Viability Analysis via ATP Assay

1. Lyopreserved samples were rehydrated and went through cell viability testing as described in Cell Viability Analysis via ATP Assay in Example 6.
2. Cryopreserved samples were thawed in 0.9% normal saline and then rinsed in 0.9% saline for 5 minutes instead of rehydration preparation, before continuing with cell viability testing as described in Cell Viability Analysis via ATP Assay above.
3. Results of cell viability testing are provided in Table 25 below.

TABLE 25
Post-Lyo & Post-Cryo Amnion/Chorion Cell Viability Results
Iteration #11

| | Post-Cryo | | Post-Lyo | |
|---|---|---|---|---|
| | AM+ | CM+ | AM+ | CM+ |
| From | 100.0% | 100.0% | 18.7% | 17.8% |
| Bilayer | | | | |
| Single | 100.0% | 100.0% | N/A | 26.4% |
| Layer | | | | |

*The Post-Lyo AM+ Single Layer sample could not be retrieved from the packaging and tested

On average the lyopreserved samples contained about 20-30% of the ATP content of the corresponding cryopreserved samples.

### Histologic Studies and Characterizations for Lyopreserved Bone Matrices

Allograft bone consisting of fresh cancellous granules and demineralized cortical fibers was aseptically processed using LyoGraft methods described herein to produce LyoGraft Bone Grafts, and then stored at ambient temperature. Cells native to the cancellous component were visualized using immunohistochemical staining for markers associated with bone-forming cells. To elucidate the preservation of endogenous proteins, multiplex ELISA arrays were used to measure analytes associated with new bone formation including ones linked to osteoinduction, and angiogenesis and osteoimmunomodulation. For the osteoimmunomodulatory panel, results were compared to demineralized cortical fiber allografts that underwent standard processing. To assess the maintenance of extracellular matrix nanostructure, thin sections of LyoGraft-processed allograft were analyzed using scanning electron microscopy.

Markers for CD166, osteocalcin and Osterix indicative of osteogenic cells were detected demonstrating that cells remained intact and adherent to the allograft following Lyograft processing. ELISA results demonstrated the presence of endogenous osteoinductive and angiogenic factors associated with bone remodeling and blood vessel development such as BMP-2, BMP-6, BMP-7, osteoprotegerin and PDGF-BB. For the osteoimmunomodulatory panel, elevated levels of proteins including TGFI(beta)-3 and IL-1(beta)] were detected for LyoGraft-processed cortico-cancellous allografts compared to standard demineralized bone fibers. These proteins are known to support crosstalk between host osteoprogenitor cells and macrophages and may shift a pro-inflammatory environment to one that is favorable for bone healing. Using SEM, collagen D-spacing was observed showcasing the preservation of natural collagen nanostructure of bone fibers after LyoGraft processing, providing pathways for cell infiltration and osteoconduction.

The LyoGraft process preserves the key bone graft properties required for fusion by maintaining the intrinsic collagenous nanostructure of the tissue, endogenous osteogenic cells and inherent osteoinductive and angiogenic factors along with proteins that may possess immunomodulatory capabilities. Additional benefits such as ambient storage, rapid preparation time and moldable handling properties provide flexibility for surgeons. In summary, LyoGraft processing of fresh allograft bone can provide a complete autograft substitute with excellent handling characteristics and simplified end-user logistics.

### LyoGraft Process for Producing LyoGraft Bone Grafts

Cancellous and cortical bone tissue samples were obtained, processed and combined, to produce LyoGraft Bone Grafts. From each donor, cancellous tissue samples comprising cancellous bone were recovered and processed to provide cancellous granules, and cortical bone tissue samples were recovered and processed to provide cortical fibers. Quantities of cancellous granules and cortical fibers derived from the same donor were measured and combined to produce three combined test samples per donor, each of which contained about 40 wt % cancellous granules and about 60 wt % cortical fibers, the wt % being based on the total weight of the combined cancellous granules and cortical fibers of each sample. The total weights of the test samples (i.e., each comprising both cancellous granules and cortical fibers) ranged from 1.3 g to 1.6 g. The test samples were preserved after contact with protectants in solution, in accordance with the methods described hereinabove.

### A. Protectant Solution Preparation:

1. Protectant solution was prepared by dissolving trehalose and EGCG into 0.9% Saline USP at concentrations of 0.4 M and 2 mM, respectively.
2. Trehalose-saline solution was prepared by dissolving trehalose into 0.9% Saline USP at a concentration of 0.4 M.
3 Following dissolution of preservative ingredients in media, both solutions were sterile filtered using a 0.2 μm membrane filter.

### B. Sample Preparation (Performed in Sterile Hood)

1. Viable cancellous tissue (granules) and demineralized cortical tissue (fibers) were obtained by sterile processing techniques as described in Example 1 above, Part B.1.
2. Bulk cancellous granules and demineralized cortical fibers were packaged under sterile conditions and temporarily stored at refrigerated temperature (2-4° C.) for a period of time greater than 0 and up to 90 minutes, until time of use in experiment.
3. In a sterile hood, cancellous granules were incubated in protectant solution for up to 1 hour.
4 After incubation, cancellous granules were combined with cortical fibers and samples aliquoted.
   a. Each sample included approximately 0.5 g cancellous granules and 0.8 g cortical fibers.
5. 3-4 mL of sterile trehalose-saline solution was added to each jar containing tissue.
6. Cortical fiber samples were aliquoted into jars without any protectant solution or trehalose-saline solution to represent standard DBF processing.
7. Jars containing tissue were placed on an aluminum or stainless steel tray and placed in the Lyopreserving Apparatus.
8. Tissue was lyopreserved generally according to the recipe provided below in Table

TABLE 26

| Thermal Treatment | Temperature (° C.) | Time (min) | Vacuum (mTorr) | Ramp/ Hold |
|---|---|---|---|---|
| Step 1: Freeze | −30 | 120 | — | Ramp |
| Freeze, Condenser, and Vacuum Parameters | Temperature (° C.) | Time (min) | Vacuum (mTorr) | Ramp/ Hold |
| Freeze Temperature (° C.) | −60 | | | Hold |
| Additional Freeze (min) | | 180 | | |
| Condenser Set Point (° C.) | −60 | | | |
| Vacuum Set Point (mTorr) | | | 500 | |
| Drying Phase Parameters | Temperature (° C.) | Time (min) | Vacuum (mTorr) | Ramp/ Hold |
| Step 1 | −5 | 60 | 200 | Ramp |
| Step 2 | −5 | 320 | 200 | Hold |
| Step 3 | −2 | 10 | 200 | Ramp |
| Step 4 | −2 | 420 | 200 | Hold |
| Step 5 | 1 | 10 | 200 | Ramp |
| Step 6 | 1 | 100 | 200 | Hold |
| Step 7 | 5 | 10 | 200 | Ramp |
| Step 8 | 5 | 280 | 200 | Hold |
| Step 9 | 10 | 10 | 200 | Ramp |
| Step 10 | 10 | 345 | 200 | Hold |
| Step 11 | 14 | 10 | 200 | Ramp |
| Step 12 | 14 | 280 | 200 | Hold |
| Secondary Drying Phase Parameters | Temperature (° C.) | Time (min) | Vacuum (mTorr) | Ramp/ Hold |
| Secondary Set Point | 15 | | | |
| Post Heat Step | 15 | 5 | 100 | Hold |

Key: “Ramp” indicates variable temperature over the time period indicated (Time (min)).
“Hold” indicates temperature held constant over the time period indicated (Time (min)).

### Example 10

### Collagen Nanostructure Analysis

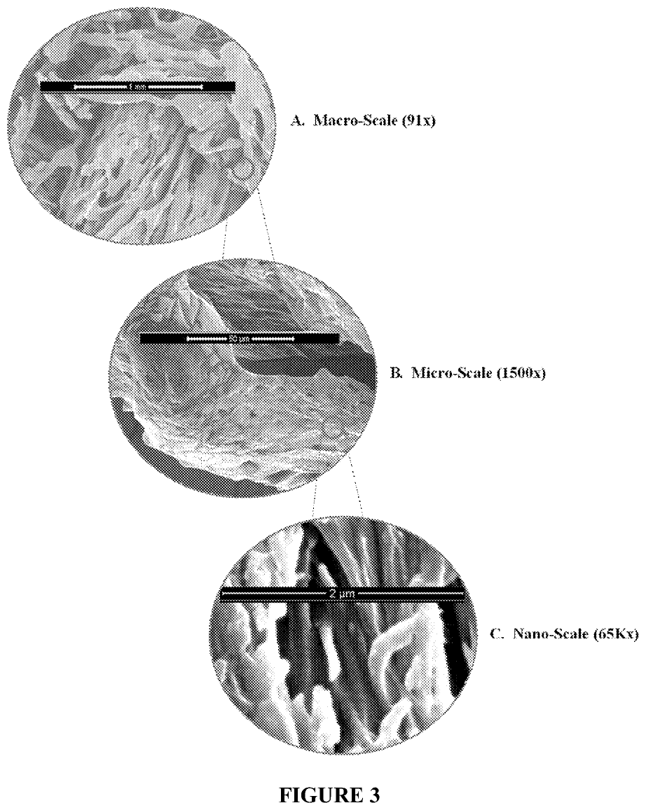

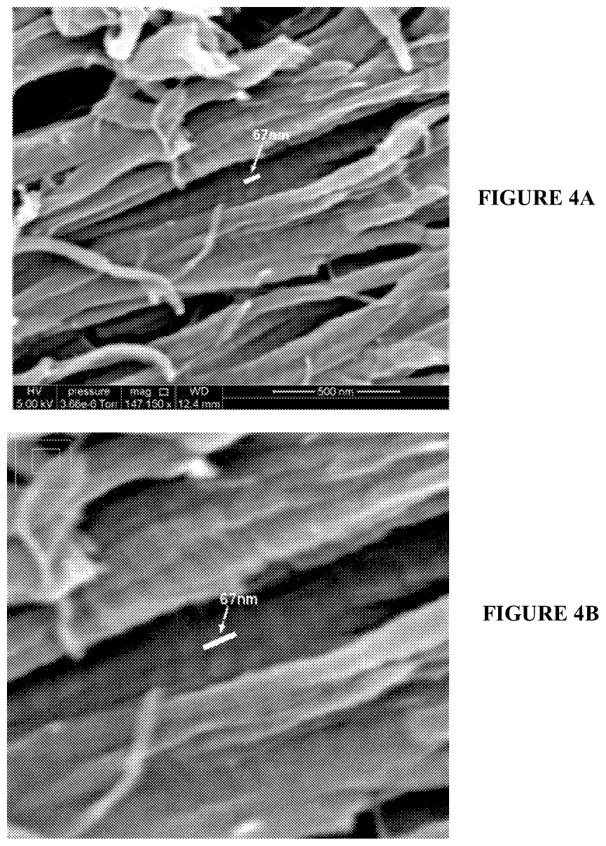

In order to visualize the collagen nanostructure after LyoGraft processing, macro and micro-scale images were first obtained by imaging the demineralized fiber component of LyoGraft Bone Grafts (~1.2 cc) with a scanning electron microscope (SEM) at the Imaging and Analysis Center (IAC) at the Princeton Institute for Science and Technology of Materials (Princeton, NJ). Entire tissue samples were affixed to a SEM stub with conductive copper tape. Then, samples were placed within the SEM and pressure was set to the low vacuum setting, a working distance of approximately 10 mm, and an accelerating voltage of 5 kV. The macro-scale and micro-scale images were magnified 91× and 1500× respectively. Astigmatism and lens alignment were corrected to sharpen the images. After the adjustments, the refresh rate was slowed down, and an image was taken. For the nano-scale image, ~0.5 g of demineralized fiber component of LyoGraft Bone Grafts was fixed in 10% neutral buffered formalin for 24 hours. After 24 hours, the sample was placed in a 1.5 mL microcentrifuge tube. An epoxy resin consisting of 20 g EMBED 812, 8 g NMA, 16 g DDSA, and 0.8 g DMP-30 was made and mixed with a wooden stir stick. The sample was dehydrated in water/ethanol solutions of increasing ethanol concentrations being increased from 25%, 50%, 75%, to 100% for 5 minutes each. Solutions were diluted with deionized water. The sample was centrifuged at 10,000 RPM for 1 minute after each dehydration step. The sample was then placed in a 1:2 epoxy resin to 100% ethanol ratio for 1 hour, 1:1 epoxy resin to 100% ethanol ratio for 1 hour, and only epoxy resin twice for 1 hour each. A new 1.5 microcentrifuge tube was obtained and a drop of resin was added to the bottom. The sample was then placed in, and the tube was filled with epoxy resin. The sample was transferred to a 60° C. oven to polymerize the resin. The sample was brought to IAC to use the Leica UCT Ultramicrotome to slice the resin and smooth the surface until the tissue was exposed. The sample was placed onto a SEM stub attached by conductive carbon tape. The Leica sputter coater was used to coat the sample with 3 nm of iridium using the tilt setting. After, the sample was imaged with the SEM using the high vacuum setting, a working distance of approximately 10 mm, and an accelerating voltage of 5 kV. The image was magnified 65,000×. Astigmatism and lens alignment were corrected to sharpen the images. After the adjustments, the refresh rate was slowed down, and an image was taken. The images produced from the foregoing procedures are provided in FIG. 3. Additional images at higher magnification that highlight the preserved inherent ~67 nm collagen bands are provided in FIGS. 4A and 4B.

### Example 11

### Immunohistochemical Staining

For this assessment, one sample of LyoGraft Bone Grafts tissue (~1.2 cc) per donor was sent to Premier Laboratory (Longmont, CO) for histological sectioning, staining, and subsequent cell count analysis after 5 months of storage in ambient conditions. Tissue was fixed in 10% neutral buffered formalin for 24-48 hrs and subsequently fitted into a cassette and decalcified for approximately 24 hrs in 10% formic acid. After decalcification, tissue was cleared with increasing concentrations of ethanol followed by a final xylene clear. Following clearing, tissue was embedded in paraffin wax and cut into 5 um slices using a microtome. Slices were placed on histology slides and dried for staining. Tissue samples were stained with H&E, CD166, Osterix, and osteocalcin stains. H&E, representing the total cell count found in the tissue form, is a combination of two histological stains, hematoxylin and eosin, which stain for cell nuclei and extracellular matrix components respectively. CD166, Osterix, and osteocalcin stain for mesenchymal stem cells, osteoprogenitor cells, and bone forming cells (osteoblast differentiation marker) respectively. The stain images for CD166+, osteocalcin, and osterix are provided in FIG. 2.

These three stains constitute the total osteogenic cell count in LyoGraft Bone Grafts. Slides were scanned using an Aperio AT2 scanner from Leica Biosystems (Buffalo Grove, IL). Based on these scans, an automated cell count was generated for each set of stained slides using a proprietary image analysis algorithm and stereologic calculation to represent cells per cc. The algorithm is based on a specific colorimetric value integrated with relative size and location considerations for an accurate assessment. Osteocalcin results were corrected for overcounting based on a regression analysis of manual vs automated cell counts established during the initial development of the cell counting algorithm. The correction factor follows the formula “Corrected Osteocalcin Count=Uncorrected Osteocalcin Count/1.2163” where 1.2163 was the slope determined by the regression analysis. Total osteogenic cell count results were corrected via an overlap correction factor (<1% overlap on average) developed by Orthofix in conjunction with Premier Laboratories to account for any commonality across the three osteogenic stains. A summary of data compiled from four unique donors is highlighted in Table 27 below:

TABLE 27

| | | | | | Total |
|---|---|---|---|---|---|
| | | | | | Osteogenic |
| | | | Osteo- | | Cell |
| | H&E | Osterix | calcin | CD166 | Count/cc |
| | Cell | Cell | Cell | Cell | with |
| Donor | Count/ | Count/ | Count/cc | Count/ | Overlap |
| ID | cc | cc | (corrected) | cc | Correction |
| A | 1,600,776 | 119,894 | 279,730 | 81,864 | 466,950 |
| B | 1,729,615 | 94,086 | 229,521 | 59,676 | 371,448 |
| C | 1,655,200 | 55,026 | 229,405 | 68,027 | 341,506 |
| D | 1,763,754 | 75,111 | 241,833 | 52,703 | 357,709 |
| Average: | 1,687,336 | 86,029 | 245,122 | 65,567 | 384,403 |

### Example 12

### Osteoimmunomodulatory Panel

To extract and evaluate proteins/cytokines in the respective tissue forms, extraction by incubating over 24 hours in cell-culture media followed by mechanical homogenization was performed. Briefly, for each donor, freeze-dried LyoGraft Bone Grafts and non-LyoGraft processed DBF tissue samples were rehydrated in saline for 2 minutes and decanted. After decanting, samples were pat until dry with gauze. After drying, ten samples of 50-60 mg were aliquoted into microcentrifuge tubes containing 8 RINO beads and 800 uL of DMEM. Each sample was thoroughly mixed and placed on a rotary shaker for 24 hours in refrigerated conditions (2-8° C.). After 24 hours, each sample was mechanically homogenized using Bullet Blender Tissue Homogenizer by NextAdvance (Troy, NY). Once complete, samples were centrifuged at 10,000 rpm for 10 minutes to separate the supernatant from the tissue debris and beads. Extracts from all 10 samples for each respective sample set were combined and vortexed to mix. Final extracts were frozen down until protein/cytokine characterization arrays were tested.

For testing, standard commercially available sandwich-based arrays for bone metabolism and human inflammation from RayBiotech were used. Each array looked at 10 proteins/cytokines of interest for a total of 20 analytes tested. Each analyte and their respective descriptions are captured in Table 28 below. The arrays were tested according to manufacturer's instructions and sent to RayBiotech for analysis.

TABLE 28

| Target | Description |
|---|---|
| BMP-2 | Bone morphogenetic protein 2 (BMP-2) Strong osteoblast differentiation factor, |
| | promotes bone growth. |
| BMP-6 | Bone morphogenetic protein 6 (BMP-6) Strong osteoblast differentiation factor, |
| | promotes bone growth. |
| BMP-7 | Bone morphogenetic protein 7 (BMP-7) Strong osteoblast differentiation factor, |
| | promotes bone growth. |
| Dkk-1 | Dickkopf-related protein 1 (Dickkopf-1) Inhibits MSC-derived |
| | osteoblastogenesis and lowers OPG. |
| MMP-3 | (Matrix metalloproteinase-3) (MMP-3) Influences bone remodeling and increases |
| | proliferation and migration of MSCs |
| OPG | Tumor necrosis factor receptor superfamily member 11B (Osteoclastogenesis |
| | inhibitory factor) (Osteoprotegerin). Decoy receptor that counterbalances effect |
| | of RANKL pathway. Downregulates osteoclast differentiation and activation |
| OPN | Osteopontin (Bone sialoprotein 1) Promotes osteoclast formation and activity. |
| PDGF-BB | Platelet-derived growth factor subunit B (PDGF subunit B) Angiogenic and |
| | mitogenic factor involved in recruitment of cells needed for bone repair. Triggers |
| | cascade of bone and soft tissue repair/regeneration |
| TGFb3 | Transforming growth factor beta-3 (TGF-beta-3). Anti-inflammatory cytokine. |
| | Promotes cell differentiation, cell adhesion and ECM formation [Cleaved into: |
| | Latency-associated peptide (LAP)] |
| TRANCE | (Osteoprotegerin ligand) (OPGL) (Receptor activator of nuclear factor kappa-B |
| | ligand) (RANKL) (TNF-related activation-induced cytokine) (TRANCE) |
| | Regulates/counterbalances OPG, promotes osteoclast formation and activation |
| IL-1a | Interleukin-1 alpha (IL-1 alpha) Strong pro-inflammatory cytokine, promotes |
| | fever and sepsis1 MSCs decrease IL-1a levels demonstrating immunomodulation. |
| IL-1b | Interleukin-1 beta (IL-1 beta) Pro-inflammatory cytokine. Linked with increased |
| | levels of MMP3 and induces macrophage type to M2b, anti-inflammatory |
| | polarization |
| IL-4 | Interleukin-4 (IL-4) plays role in mitigating chronic inflammation and promoting |
| | wound repair. Promotes M2 macrophage activation. Inhibits osteoclast formation. |
| IL-6 | Interleukin-6 (IL-6) Strong pro-inflammatory cytokine, promotes increased |
| | osteoclast activity. |
| IL-8 | Interleukin-8 (IL-8) Strong pro-inflammatory cytokine, promotes increased |
| | osteoclast activity. |
| IL-10 | Interleukin-10 (IL-10) anti-inflammatory cytokine, maintains bone mass through |
| | inhibition of osteoclasts |
| IL-13 | Interleukin-13 (IL-13) suppresses bone resorption activity of IL-1a, inhibits cell |
| | proliferation. |
| MCP-1 | Monocyte chemotactic protein 1 (aka CCL2) Exhibits chemotactic activity for |
| | monocytes and is linked to formation of osteoclasts |
| IFNg | Interferon gamma (IFN-gamma) (Immune interferon) Strong pro-inflammatory |
| | cytokine, induces production of more RANKL and TNF-α to promote bone |
| | resorption/erosion |
| TNFa | Tumor necrosis factor (Cachectin) (TNF-alpha) Strong pro-inflammatory |
| | cytokine, promotes increased osteoclast activity. |

From the results, it was evident that LyoGraft Bone Grafts has several key proteins that have different profiles compared to lyophilized DBF without cells. These proteins are associated with down-stream anti-inflammatory, pro-anabolic, and anti-catabolic pathways. A brief summary of the findings for each panel are highlighted in Table 29 and 30. The “higher/lower” designations for these tables are attached to analytes with at least a 30% difference between LyoGraft Bone Grafts & DBF (detailed breakdown of data located in Table 31). Analytes with less than 30% difference were designated as “same”. Osteoprotegerin (OPG), osteopontin (OPN), platelet derived growth factor subunit B (PDGF-BB), transforming growth factor beta-3 (TGFb3), and interleukin-1beta (IL-1b) were five proteins of interest that were highlighted and showed higher/lower relative detection levels of at least 30% consistently across 3 donors lots. These proteins are known to influence bone healing response based on in vitro and in vivo studies, supported by peer-reviewed literature.

TABLE 29
Data summary table for bone metabolism array results for all three donors.

| | Bone Metabolism (pg/g) | | | | | | | | |
|---|---|---|---|---|---|---|---|---|---|
| | Donor 1 | | | Donor 2 | | | Donor 3 | | |
| | Lyograft | DBF | Lyograft vs DBF | Lyograft | DBF | Lyograft vs DBF | Lyograft | DBF | Lyograft vs DBF |
| OPG | + | − | Higher | + | − | Higher | + | − | Higher |
| OPN | + | + | Lower | + | + | Lower | + | + | Lower |
| PDGF-BB | + | + | Higher | + | − | Higher | + | − | Higher |
| TGFb3 | + | − | Higher | + | + | Higher | + | + | Higher |

NOTE:
(+) and (−) denote detection of each respective protein. The qualifier for higher/lower designation is at least a 30% difference in LyoGraft Bone Grafts vs DBF.

TABLE 30
Data summary table for human inflammation array results for all three donors.

| | Human Inflammation (pg/g) | | | | | | | | |
|---|---|---|---|---|---|---|---|---|---|
| | Donor 1 | | | Donor 2 | | | Donor 3 | | |
| | Lyograft | DBF | Lyograft vs DBF | Lyograft | DB | Lyograft vs DBF | Lyograft | DBF | Lyograft vs DBF |
| IL-1b | + | + | Higher | + | − | Higher | + | + | Higher |

(+) and (−) denote detection of each respective protein. The qualifier for higher/lower designation is at least a 30% difference in LyoGraft Bone Grafts (RTT) vs DBF.

Table 31 below contains standardized results for each analyte (growth factor or other protein) of interest. Prior to standardization, the minimum and maximum values of each analyte across all test groups were identified. In order to calculate the range of values for each analyte, the minimum value was subtracted from the maximum value. Standardization was performed using the following formula:

$$Analyte ( standardized ) = ( Result value - Minimum Value ) / Range$$

TABLE 31
Standardized Results for each analyte (growth
factor or other protein), RTT v DBF

| Analyte | Donor 1 | | Donor 2 | | Donor 3 | | Average | |
|---|---|---|---|---|---|---|---|---|
| (pg/g) | RTT | DBF | RTT | DBF | RTT | DBF | RTT | DBF |
| TGFb3 | 1.00 | 0.00 | 0.23 | 0.09 | 0.36 | 0.25 | 0.53 | 0.11 |
| OPG | 1.00 | 0.00 | 0.08 | 0.00 | 0.90 | 0.00 | 0.66 | 0.00 |
| OPN | 0.09 | 0.90 | 0.00 | 0.60 | 0.09 | 1.00 | 0.06 | 0.83 |
| PDGF-BB | 1.00 | 0.28 | 0.11 | 0.00 | 0.26 | 0.00 | 0.45 | 0.09 |
| IL-1b | 1.00 | 0.43 | 0.11 | 0.00 | 0.37 | 0.17 | 0.49 | 0.20 |

Results compared to demineralized bone fibers are indicated in Table 32 with a standardized results key highlighted in Table 33.

TABLE 32

| | n = 3 standardized avg. | |
|---|---|---|
| Analyte | (RTF) | DBF |
| TGFb3 | +++ | + |
| OPG | +++ | − |
| PDGF-BB | ++ | + |
| IL-1B | ++ | + |

TABLE 33
Standardized
Results Key

| | |
|---|---|
| 0.00 | − |
| .01-.24 | + |
| .25-.49 | ++ |
| .50-.74 | +++ |
| .75-1.00 | ++++ |

### Example 13

Effect of Pre-Processing Base Solution and pH on Cell Viability after Lyopreservation and Rehydration

For this experiment, base solutions of varying pH and salt concentrations were assessed in the preparation of lyoprotectant solution. Color of solution, post-lyophilization color of tissue, and cell viability pre and post-lyophilization were assessed. Cancellous and cortical bone tissue samples from a single donor were obtained, processed, and combined using the methods described below to produce 36 combined test samples. Three different base solutions were assessed, combining 0.4 M trehalose and 4 mM EGCG into PBS, Saline, and HBSS. The pH of each solution was tested and color assessed. Each solution set was used to process combined samples of cancellous and cortical bone tissue and tested for cell viability before and after lyophilization. Cell viability was assessed by measuring ATP, with results reported below based on the mathematical average of three test samples from each test group derived from a common donor.

### A. Protectant Solution Preparation

1. Protectant solution was prepared by dissolving trehalose and EGCG into 1×PBS, 0.9% Sodium Chloride USP, and 1×HBSS (Hank's Balanced Salt Solution) at concentrations of 0.4 M and 4 mM respectively.
2. Following dissolution of preservative ingredients in media, the solution was sterile filtered using a 0.2 μm membrane filter.

### B. Sample Preparation (Performed in Sterile Hood)

3. Viable cancellous tissue (granules) and demineralized cortical tissue (fibers) were obtained by sterile processing techniques as described in Example 1 above, Part B.1.
4. Bulk cancellous granules and demineralized cortical fibers were packaged under sterile conditions and temporarily stored at refrigerated temperature (2-4° C.) for a period of time greater than 0 and up to 90 minutes, until time of use in experiment.
5. In a sterile hood, bulk cancellous granules and demineralized cortical fibers were mixed and homogenized.
   5.1 Multiple samples were weighed out into 1 oz. jars, with each sample having approximately 0.5 g cancellous granules and 0.8 g cortical fibers.
6. 4 mL of sterile protectant solution was added to each jar containing tissue. 12 samples were prepared per protectant solution type (PBS, Saline, HBSS) for a total of 36 samples.
7. Tissue was allowed to incubate with protectant solution at room temperature for 1 hour.
8. After the incubation period, jars containing tissue were placed on an aluminum or stainless steel tray and placed in the Lyopreserving Apparatus.
9. Tissue was lyopreserved generally according to the recipe provided below in Table 33.

TABLE 33

| Recipe (30 hrs) | | | | |
|---|---|---|---|---|
| Thermal Treatment | Temperature (° C.) | Time (min) | Vacuum (mTorr) | Ramp/Hold |
| Freeze Parameters | −30 | 120 | — | Ramp |
| Freeze, Condensor, and Vaccum Parameters | Temperature (° C.) | Time (min) | Vacuum (mTorr) | Ramp/Hold |
| Freeze Temperature (°C) | −30 | | | Hold |
| Additional Freeze (min) | | 180 | | |
| Condensor Set Point (°C) | −60 | | | |
| Vacuum Set Point (mTorr) | | | 500 | |
| Drying Phase Parameters | Temperature (° C.) | Time (min) | Vacuum (mTorr) | Ramp/Hold |
| Step 1 | −15 | 40 | 200 | Ramp |
| Step 2 | −15 | 1000 | 200 | Hold |
| Step 3 | 1 | 60 | 200 | Ramp |
| Step 4 | 1 | 600 | 200 | Hold |
| Secondary Drying Phase Parameters | Temperature (° C.) | Time (min) | Vacuum (mTorr) | Ramp/Hold |
| Secondary Set Point | 2 | | | |
| Post Heat Step | 2 | 5 | 100 | Hold |

### C. Solution Testing

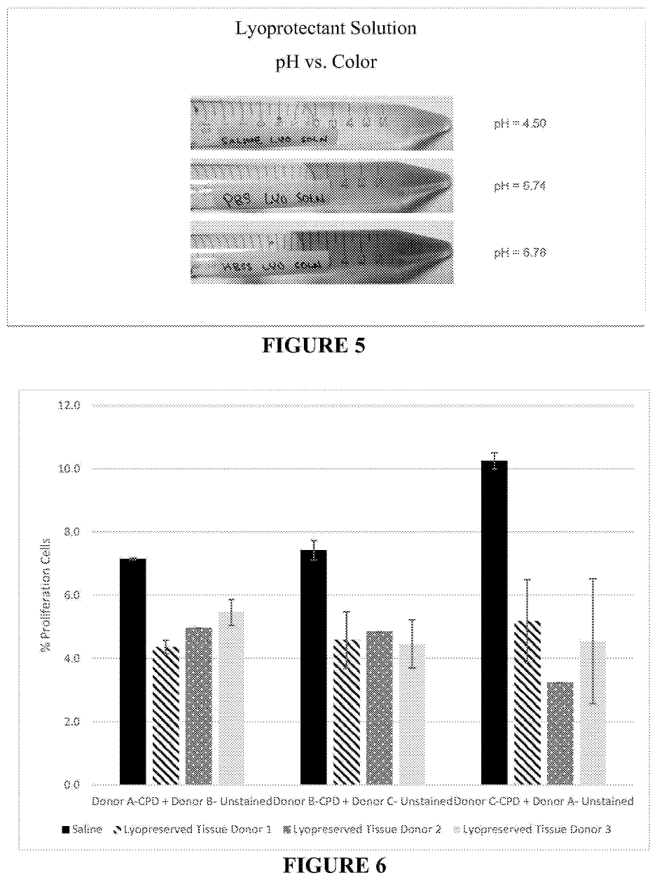

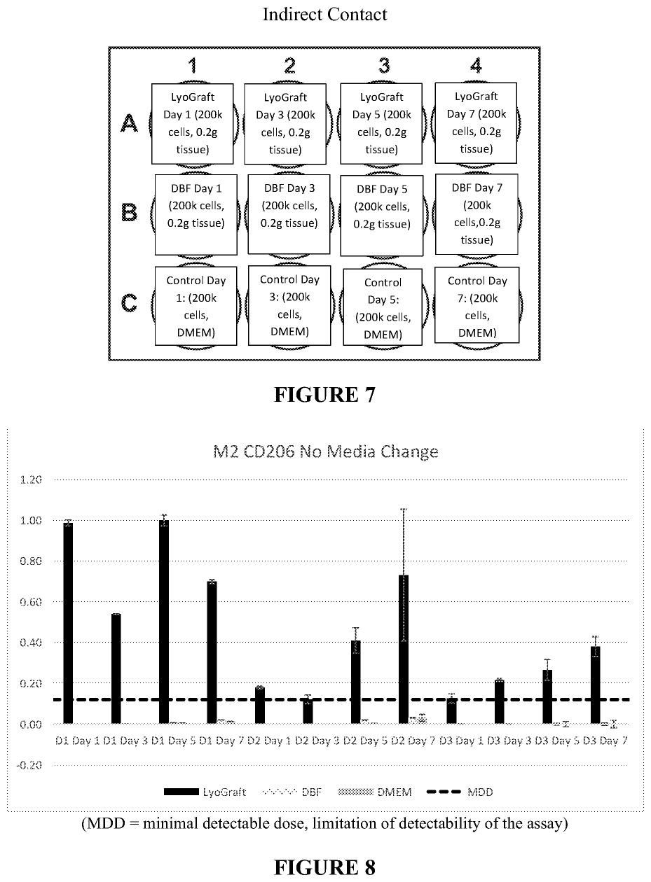

10. PBS, Saline, and HBSS base solutions were kept for 24 hours at room temperature and monitored for color changes and tested for pH using a pH meter. pH and solution color results are presented in FIG. 5.
   As shown in FIG. 5, it appeared that the color change of the lyoprotectant solution was associated with the type of base solution. A darker color hue was related to higher pH values. Therefore, without wishing to be bound by theory, it was hypothesized that the type of solution as well as the pH may result in a chemical reaction that causes EGCG to degrade, which would in turn directly affect cell viability.

### D. Sample Testing

11. Prior to lyopreservation but post-contact and incubation with the protectant solution (i.e., prior to step B8 above), one or more test samples containing about 1.3-1.6 g of total tissue were measured and tested per the ATP viability assay procedures described above, which provided “Pre-Lyo” (i.e., “post-contact and incubation”) ATP values.
12. Following lyopreserving, samples were rehydrated in 1×PBS, 0.9% Saline USP, and 1×HBSS for each base solution condition. Solution was added to the fill line of each jar and letting sit at room temperature for 5 minutes.
13. Following the 5 minute rehydration period, excess solution was decanted from all jars.
14. For each sample, all the tissue (1.3-1.6 g) was tested per the ATP viability assay procedures described above and values corrected based on their pre-lyo weight. Table 34 and 35 below captures the results. Results for both tables were normalized according to the highest average ATP values, in this case Pre-Lyo Saline Lyo Soln. All results are presented as a percentage of the Pre-Lyo Saline Lyo Soln ATP content, according to the following formula:

$$Normalized ATP = Result / Average Pre - Lyo Saline Lyo Soln ATP$$

TABLE 34

| | Pre-Lyophilization ATP | | | | |
|---|---|---|---|---|---|
| | | | | Average | Std. |
| Condition | Sample 1 | Sample 2 | Sample 3 | ATP | Dev. |
| PBS Lyo Soln | 0.74 | 0.70 | 0.69 | 0.69 | 0.025 |
| Saline Lyo Soln | 0.90 | 1.06 | 1.00 | 1.00 | 0.083 |
| HBSS Lyo Soln | 0.86 | 0.81 | 0.83 | 0.83 | 0.025 |

TABLE 35

| | Post-Lyophilization ATP | | | | | | | | | | |
|---|---|---|---|---|---|---|---|---|---|---|---|
| | Reconstitute in PBS | | | Reconstitute in Saline | | | Reconstitute in HBSS | | | | |
| | Sample | Sample | Sample | Sample | Sample | Sample | Sample | Sample | Sample | Average | Std. |
| Condition | 1 | 2 | 3 | 4 | 5 | 6 | 7 | 8 | 9 | ATP | Dev. |
| PBS Lyo Soln | 0.51 | 0.42 | 0.56 | 0.51 | 0.45 | 0.56 | 0.59 | 0.59 | 0.54 | 0.53 | 0.06 |
| Saline Lyo Soln | 0.66 | 0.72 | 0.88 | 0.74 | 0.64 | 0.84 | 0.70 | 0.64 | 0.84 | 0.74 | 0.09 |
| HBSS Lyo Soln | 0.52 | 0.56 | 0.87 | 0.68 | 0.74 | 0.73 | 0.57 | 0.52 | 0.67 | 0.65 | 0.12 |

For the ATP values, the samples with saline-based lyophilization solution were highest during pre-lyophilization and post-lyophilization. The percent differences for saline with respect to (wrt) PBS and HBSS, respectively, are provided in Table 36 below.

TABLE 36

| | Saline Lyo Solution Viability Increase |
|---|---|
| % Difference wrt PBS | 40% |
| % Difference wrt HBSS | 14% |

This data indicates that saline was superior with resect to (wrt) to PBS and HBSS as the base media in lyopreservation solution by 40% and 13%, respectively. The percent values reported in Table 36 above were calculated using the following formulas:

$$\% Difference w r t PBS = \frac{( Average ATP Saline - Average ATP PBS )}{Average ATP PBS} X 100$$

$$\% Difference w r t HBSS = \frac{( Average ATP Saline - Average ATP HBSS )}{Average ATP HBSS} X 100$$

On average, there was a 27% increase in cell viability using saline as the base media for the lyopreservation solution compared to the other two test groups. This was calculated by taking the average of the two results captured in Table 36 above.

### Example 14

### Mixed Lymphocyte Reaction (MLR) Testing of Lyopreserved Cancellous and Cortical Tissue

Cancellous and cortical bone tissue samples were obtained, processed and combined, to produce LyoGraft Bone Grafts. From each donor, cancellous tissue samples comprising cancellous bone were recovered and processed to provide cancellous granules, and cortical bone tissue samples were recovered and processed to provide cortical fibers. Quantities of cancellous granules and cortical fibers derived from the same donor were measured and combined to produce three test samples per donor, each of which contained about 40 wt % cancellous granules and about 60 wt % cortical fibers, the wt % being based on the total weight of the combined cancellous granules and cortical fibers of each sample. The total weights of the test samples (i.e., each comprising both cancellous granules and cortical fibers) ranged from 1.3 g to 1.6 g.

The test samples were preserved after contact with protectants in solution, in accordance with the methods described hereinabove. Peripheral blood mononuclear cells (PBMCs) were procured from three unique blood donors in order to test for a one-way and two-war mixed lymphocyte reaction (MLR) assay. In a one-way set up, PBMCs are exposed to a test article, the lyopreserved tissue samples, and PBMC stimulation or lack thereof is assessed. In a two-way set up, mismatched blood donors are exposed to one another, and an ongoing reaction is stimulated. This ongoing reaction is then put in presence of a test article, the lyopreserved tissue samples, and stimulation or inhibition of this ongoing reaction are assessed.

One-way MLR testing was performed on three lyopreserved donor lots to show that LyoGraft Bone Grafts does not elicit cell proliferation/stimulation when added to peripheral blood mononuclear cells (PBMCs), tested against three unique blood donors.

Two-way MLR testing was performed on three lyopreserved donor lots to assess the inhibitory or stimulatory effects of an ongoing/developing immunogenic reaction between two mismatched blood donors.

Final results indicate that LyoGraft Bone Grafts are non-immunogenic and have an inhibitory effect on an ongoing immunogenic response between two mismatched PBMC donors.

### A. Protectant Solution Preparation:

1. Protectant solution was prepared by dissolving trehalose and EGCG into 0.9% Saline USP at concentrations of 0.4 M and 2 mM, respectively.
2. Trehalose-saline solution was prepared by dissolving trehalose into 0.9% Saline USP at a concentration of 0.4 M.
3. Following dissolution of preservative ingredients in media, both solutions were sterile filtered using a 0.2 μm membrane filter.

### B. Sample Preparation (Performed in Sterile Hood)

4. Viable cancellous tissue (granules) and demineralized cortical tissue (fibers) were obtained by sterile processing techniques as described in Example 1 above, Part B.1.
5. Bulk cancellous granules and demineralized cortical fibers were packaged under sterile conditions and temporarily stored at refrigerated temperature (2-4° C.) for a period of time greater than 0 and up to 90 minutes, until time of use in experiment.
6. In a sterile hood, cancellous granules were incubated in protectant solution for up to 1 hour.
7. After incubation, cancellous granules were combined with cortical fibers and samples aliquoted.
   7.1 Each sample from Donors 1 and 3 included approximately 0.5 g cancellous granules and 0.8 g cortical fibers.
   7.2 Larger samples processed from Donor 2 included approximately 2.2 g cancellous granules and 3.3 g cortical fibers, maintaining the same ratio of cancellous granules to cortical fibers as Donor 1 and 3.
8. 3-4 mL of sterile trehalose-saline solution was added to each jar containing tissue.
9. Jars containing tissue were placed on an aluminum or stainless steel tray and placed in the Lyopreserving Apparatus.
10. Tissue was lyopreserved generally according to the recipe provided in Table 26 (see Example 9 above).

### C. One-Way MLR

11. Blood was obtained from three unique donors and PBMCs were isolated.
12. PBMCs were stained with a tracking dye, CPD-670, and added to 96-well round bottom culture plates for one-way MLR assay.
   12.1 This dye is used to determine whether or not an immune response has been induced. If there is an immune reaction, cells will proliferate in response to a stimulator, in this case lyopreserved tissue, and dilute the CPD-670 stain. The dilution of the stain decreases the fluorescence signal which will be used to measure any differences across test samples compared to the saline negative control and concanavalin A positive control.
13. Lyopreserved samples are prepared by adding 30 mL of sterile saline USP to smaller units (Donor 1 and 3) or filling 2 oz jar to the top (60 mL) with saline for larger units (Donor 2), incubating for 2 mins, decanting, and tap-drying with sterile gauze.
14. 60 mg of lyopreserved tissue per donor and 20 uL of saline were added in tripliciate for each PBMC donor in 96 well plates.
   14.1 Saline is used as a negative control.
15. PBMCs were added to each test group (250K cells) and incubated for 5 days at 37° C. in 5% CO2 in humidifier incubator.
16. To calculate % proliferation, cells with decreased dye fluorescent signal were compared to the total number of positively stained cells.
17. Concanavalin A (ConA) was used as a stimulation positive control to ensure that cells (PBMC) were in good condition to proliferate.
18. Stimulation Index (SI) was calculated as proliferating cell percentage from wells with PBMCs+lyopreserved tissue sample divided by proliferating cell percentage from PBMCs+saline. Results are outlined in Tables 37A, 37B and 37C below, and are evidence that the lyopreserved test units do not illicit an immunogenic response.
   18.1 A stimulation index <1.5 is considered non-immunogenic.

TABLE 37A
Proliferated cells (% of all CPD stained)-Donor A

| | | | | Stimu- |
|---|---|---|---|---|
| | | % | | lation |
| Donor A One Way MLR | Average | CV | StdDev | Index |
| Donor A + Saline | 1.6 | 13% | 0.2 | — |
| Donor A + Lyopreserved Donor #1 | 0.5 | 16% | 0.1 | 0.3 |
| Donor A + Lyopreserved Donor #2 | 0.5 | 22% | 0.1 | 0.3 |
| Donor A + Lyopreserved Donor #3 | 0.4 | 17% | 0.1 | 0.2 |
| Donor A + ConA Positive Control | 45.2 | 6% | 2.6 | 28.0 |

TABLE 37B
Proliferated cells (% of all CPD stained)-Donor B

| | | | | Stimu- |
|---|---|---|---|---|
| | | % | | lation |
| Donor B One Way MLR | Average | CV | StdDev | Index |
| Donor B + Saline | 0.6 | 30% | 0.2 | — |
| Donor B + Lyopreserved Donor #1 | 0.1 | 23% | 0.0 | 0.2 |
| Donor B + Lyopreserved Donor #2 | 0.1 | 87% | 0.1 | 0.1 |
| Donor B + Lyopreserved Donor #3 | 0.1 | 41% | 0.0 | 0.1 |
| Donor B + ConA Positive Control | 61.5 | 2% | 1.1 | 100.3 |

TABLE 37C
Proliferated cells (% of all CPD stained)-Donor C

| | | | | Stimu- |
|---|---|---|---|---|
| | | % | | lation |
| Donor C One Way MLR | Average | CV | StdDev | Index |
| Donor C + Saline | 0.4 | 31% | 0.1 | — |
| Donor C + Lyopreserved | 0.2 | 24% | 0.0 | 0.4 |
| Donor #1 | | | | |
| Donor C + Lyopreserved | 0.2 | 52% | 0.1 | 0.5 |
| Donor #2 | | | | |
| Donor C + Lyopreserved | 0.1 | 26% | 0.0 | 0.3 |
| Donor #3 | | | | |
| Donor C + ConA Positive | 60.6 | 1% | 0.4 | 155.4 |
| Control | | | | |

### D. Two-Way MLR

19. Cells (PBMC) from each of two donors were added to wells of 96-well round bottom culture plates.
20. Only cells from one of the two donors were stained with tracking dye, CPD-670, considered the responder cells. The other donor cells were left unstained, considered the inducer cells.
21. Assay was repeated using 3 different combinations of responder/inducer cells.
22. The amount of proliferating responder cells indicates an immune reaction induced by the mismatched inducer cells and is shown as cells with reduced dye content after six days of co-incubation.
23. Virtuos samples are prepared by adding 30 mL of sterile saline USP to smaller units (Donor 1 and 3) or filling 2 oz jar to the top (60 mL) with saline for larger units (Donor 2), incubating for 2 mins, decanting, and tap-drying with sterile gauze.
24. 60 mg of lyopreserved tissue per donor and 20 uL of saline were added in triplicate for each PBMC donor in 96 well plates.
   24.1 Saline is used as a negative control.
25. PBMCs from two mismatched blood donors were added to each test group (250K cells per blood donor) and incubated for 5 days at 37° C. in 5% CO₂ in humidifier incubator.
26. Concanavalin A (ConA) was used as a stimulation positive control to ensure that cells (PBMC) were in good condition to proliferate.
27. Stimulation Index (SI) was calculated as proliferating cell percentage from wells with two mismatched donors with lyopreserved tissue divided by proliferating cell percentage from wells with the same mismatched donor cells treated with saline. Results are outlined in Tables 38A, 38B, and 38C, and are evidence that the lyopreserved test units impact an inhibitory effect in an ongoing immunogenic reaction between mismatched sets of PBMCs.

TABLE 38A
Proliferated cells (% of all CPD stained)-Donor A

| | | | | Stimu- | Inhibi |
|---|---|---|---|---|---|
| | | % | | lation | -tion |
| Donor A Two Way MLR | Average | CV | StdDev | Index | % |
| Donor A + Saline | 1.6 | 13% | 0.2 | — | — |
| Donor A-CPD + Donor B- | 7.2 | 0% | 0.0 | 4.43 | — |
| Unstained MLR + Saline | | | | | |
| Donor A-CPD + Donor B- | 4.4 | 5% | 0.2 | 0.6 | 38.9 |
| Unstained MLR + | | | | | |
| Lyopreserved Donor #1 | | | | | |
| Donor A-CPD + Donor B- | 5.0 | — | — | 0.7 | 30.5 |
| Unstained MLR + | | | | | |
| Lyopreserved Donor #2 | | | | | |
| Donor A-CPD + Donor B- | 5.5 | 8% | 0.4 | 0.8 | 23.6 |
| Unstained MLR + | | | | | |
| Lyopreserved Donor #3 | | | | | |
| Donor A-CPD + Donor B- | 57.8 | 3% | 1.7 | 8.1 | |
| Unstained MLR + ConA | | | | | |
| Control | | | | | |

TABLE 38B
Proliferated cells (% of all CPD stained)-Donor B

| | | | | Stimu- | Inhibi- |
|---|---|---|---|---|---|
| | | % | | lation | tion |
| Donor B Two Way MLR | Average | CV | StdDev | Index | % |
| Donor B + Saline | 0.6 | 30% | 0.2 | — | — |
| Donor B-CPD + Donor C- | 7.4 | 4% | 0.3 | 12.10 | — |
| Unstained MLR + Saline | | | | | |
| Donor B-CPD + Donor C- | 4.6 | 20% | 0.9 | 0.6 | 38.2 |
| Unstained MLR + | | | | | |
| Lyopreserved Donor #1 | | | | | |
| Donor B-CPD + Donor C- | 4.9 | — | — | 0.7 | 34.5 |
| Unstained MLR + | | | | | |
| Lyopreserved Donor #2 | | | | | |
| Donor B-CPD + Donor C- | 4.5 | 17% | 0.8 | 0.6 | 39.8 |
| Unstained MLR + | | | | | |
| Lyopreserved Donor #3 | | | | | |
| Donor B-CPD + Donor C- | 39.5 | 1% | 0.4 | 5.3 | |
| Unstained MLR + ConA | | | | | |
| Control | | | | | |

TABLE 38C
Proliferated cells (% of all CPD stained)-Donor C

| | | | | Stimu- | Inhibi- |
|---|---|---|---|---|---|
| | | % | | lation | tion |
| Donor C Two Way MLR | Average | CV | StdDev | Index | % |
| Donor C + Saline | 0.4 | 31% | 0.1 | — | — |
| Donor C-CPD + Donor A- | 10.3 | 3% | 0.3 | 26.28 | — |
| Unstained MLR + Saline | | | | | |
| Donor C-CPD + Donor A- | 5.2 | 25% | 1.3 | 0.5 | 49.3 |
| Unstained MLR + | | | | | |
| Lyopreserved Donor #1 | | | | | |
| Donor C-CPD + Donor A- | 3.3 | — | — | 0.3 | 68.3 |
| Unstained MLR + | | | | | |
| Lyopreserved Donor #2 | | | | | |
| Donor C-CPD + Donor A- | 4.5 | 44% | 2.0 | 0.4 | 55.6 |
| Unstained MLR + | | | | | |
| Lyopreserved Donor #3 | | | | | |
| Donor C-CPD + Donor A- | 50.6 | 1% | 0.3 | 4.9 | |
| Unstained MLR + ConA | | | | | |
| Control | | | | | |

### Example 15

### Macrophage Polarization in Presence of LyoGraft Bone Grafts

Cancellous and cortical bone tissue samples were obtained, processed and combined, to produce LyoGraft Bone Grafts. From each donor, cancellous tissue samples comprising cancellous bone were recovered and processed to provide cancellous granules, and cortical bone tissue samples were recovered and processed to provide cortical fibers. Quantities of cancellous granules and cortical fibers derived from the same donor were measured and combined to produce three test samples per donor, each of which contained about 40 wt % cancellous granules and about 60 wt % cortical fibers, the wt % being based on the total weight of the combined cancellous granules and cortical fibers of each sample. The total weights of the test samples (i.e., each comprising both cancellous granules and cortical fibers) ranged from 1.3 g to 1.6 g.

The test samples were preserved after contact with protectants in solution, in accordance with the methods described hereinabove. Demineralized bone fibers from the same donor were retrieved and lyophilized without protectants in the same weight ranges as the combined cancellous granules and cortical fibers comprising LyoGraft Bone Grafts. THP-1 Monocytes were cultured and differentiated into MO macrophages using phorbol 12-myristate 13-acetate (PMA) and seeded in presence of LyoGraft Bone Grafts, demineralized bone fibers, and DMEM/media negative control group to assess polarization. Polarization of MO macrophages was determined via ELISA for pro-healing, anti-inflammatory M2 marker CD206/Mannose. All processing, culturing, and testing steps are elucidated below.

### A. Protectant Solution Preparation:

1. Protectant solution was prepared by dissolving trehalose and EGCG into 0.9% Saline USP at concentrations of 0.4 M and 2 mM, respectively.
2. Trehalose-saline solution was prepared by dissolving trehalose into 0.9% Saline USP at a concentration of 0.4 M.
3. Following dissolution of preservative ingredients in media, both solutions were sterile filtered using a 0.2 μm membrane filter.

### B. Sample Preparation (Performed in Sterile Hood)

4. Viable cancellous tissue (granules) and demineralized cortical tissue (fibers) were obtained by sterile processing techniques as described in Example 1 above, Part B.1.
5. Bulk cancellous granules and demineralized cortical fibers were packaged under sterile conditions and temporarily stored at refrigerated temperature (2-4° C.) for a period of time greater than 0 and up to 90 minutes, until time of use in experiment.
6. In a sterile hood, cancellous granules were incubated in protectant solution for up to 1 hour.
7. After incubation, cancellous granules were combined with cortical fibers and samples aliquoted.
   a. Each sample included approximately 0.5 g cancellous granules and 0.8 g cortical fibers.
8. 3-4 mL of sterile trehalose-saline solution was added to each jar containing tissue.
9. Separately, 1.3-1.6 g of demineralized bone fibers from the same donor were aliquoted into jars and prepared for lyophilized.
10. Jars containing tissue samples were placed on an aluminum or stainless steel tray and placed in the Lyopreserving Apparatus.
11. Tissue was lyopreserved generally according to the recipe provided in Table 26 (see Example 9 above).

### C. THP-1 Monocyte Preparation and Polarization to MO Macrophages

12. Fetal Bovine Serum and penicillin/streptomycin is added to RPMI-1640 media in 10% and 1% amounts by volume respectively to create complete RPMI media.
13. THP-1 Monocytes are received cryopreserved. Cells are quickly thawed in a 37° C. water bath and subsequently passed into a biosafety cabinet. All subsequent processing steps are performed under sterile conditions.
14. In the cryovial, cell suspension was pipetted up and down with a 1000 uL pipette to homogenize.
15. The lot specific information (number of cells and expected viability) from the Certificate of Analysis (COA) was leveraged to calculate the volume of medium necessary to start the THP-1 cell culture at the recommended seeding density of 3×10\hat{ }5 viable cells/ml.
16. Cell suspension was transferred to a 15 mL tube containing 10 mL of complete culture medium and homogenized, centrifuged, and media aspirated to retain a cell pellet.
17. Cells were resuspended in 3-5 mL of complete media and using a hemocytometer, cell counts were calculated. Cell dilution was adjusted to density of 3×10\hat{ }5 cells/mL.
18. Cells were plated onto T75 flasks and cultured until an adequate amount of cells were present for differentiation and seeding.
   18.1 Media was changed every 2-3 days. Cell density did not exceed 1.5×10\hat{ }6 cells/mL, either by adding more media to dilute cellular concentration or monocytes were passaged into additional T75 flasks.
19. Once an adequate amount of cells were present, PMA was prepared by initially dissolving in DMSO. PMA-DMSO was subsequently diluted in complete RPMI media and added to the cells in a working concentration of 100 nM.
20. THP-1 Monocytes were differentiated into MO Macrophages over 24 hours in 37 C, 5% CO2 incubator in presence of 100 nM PMA.
21. MO macrophages were prepared for indirect seeding/culture experiment according to the follow set up:
   Indirect Contact: Tissue+cells separated by transwell
   Transwell set up:
      3 um transwell
      0.2 g tissue in transwell per condition
      500 uL complete RPMI-1640 on top of transwell
   Well plate set up:
      1500 uL complete RPMI-1640 in well plate (133K cells/mL concentration)
      200K cells per plate
22. All test groups were incubated at 37 C in 5% CO₂ incubator and media extracts were tested at 24 hr, 72 hr, 120 hr, and 168 hr timepoints

### D. Sample Collection/Assessments

23. At each timepoint, morphology of the cells in each well plate (indirect contact) was assessed and representative images were taken.
   23.1 At each timepoint, media was removed from each test group for ELISA testing.
   23.2 All solution was removed (~1-1.2 mL) and dispensed into appropriately labeled tubes.
   23.3 For Mannose/CD206 testing, Human Mannose Receptor ELISA kit (ab277420) from Abcam was leveraged. All testing followed manufacturer's instructions. Each seeding condition and timepoint was tested in triplicate with results presented as an average.
      i. Results indicate that LyoGraft test group has osteoimmunomodulatory properties, having higher M2 markers after co-culture with MO macrophages. Standardized results are outlined in Table 39 below and shown in a bar graph in FIG. 8. Prior to standardization, the minimum and maximum values of each analyte average across all test groups were identified. In order to calculate the range of values for each analyte, the minimum value was subtracted from the maximum value. Standardization was performed using the following formula:

$$Standardized Average \frac{( Result value - Minimum Value )}{Range} \times 100$$

TABLE 39
Standardized results of M2/CD206 ELISA results

| Donor 1 CD206 | | | | Donor 2 CD206 | | | | Donor 3 CD206 | | | |
|---|---|---|---|---|---|---|---|---|---|---|---|
| Condition: | | | | Condition: | | | | Condition: | | | |
| No Media | Time- | Standardized | Standard | No Media | Time- | Standardized | Standard | No Media | Time- | Standardized | Standard |
| Change | point | Average | Deviation | Change | point | Average | Deviation | Change | point | Average | Deviation |
| LyoGraft | Day 1 | 0.99 | 0.01 | LyoGraft | Day 1 | 0.18 | 0.01 | LyoGraft | Day 1 | 0.13 | 0.02 |
| | Day 3 | 0.54 | 0.00 | | Day 3 | 0.12 | 0.02 | | Day 3 | 0.22 | 0.01 |
| | Day 5 | 1.00 | 0.03 | | Day 5 | 0.41 | 0.06 | | Day 5 | 0.27 | 0.05 |
| | Day 7 | 0.70 | 0.01 | | Day 7 | 0.73 | 0.32 | | Day 7 | 0.38 | 0.05 |
| DBF | Day 1 | 0.00 | 0.00 | DBF | Day 1 | 0.00 | 0.00 | DBF | Day 1 | 0.00 | 0.00 |
| | Day 3 | 0.00 | 0.00 | | Day 3 | 0.00 | 0.00 | | Day 3 | 0.00 | 0.00 |
| | Day 5 | 0.01 | 0.00 | | Day 5 | 0.02 | 0.00 | | Day 5 | 0.00 | 0.00 |
| | Day 7 | 0.02 | 0.00 | | Day 7 | 0.03 | 0.00 | | Day 7 | 0.00 | 0.01 |
| DMEM | Day 1 | 0.00 | 0.00 | DMEM | Day 1 | 0.00 | 0.00 | DMEM | Day 1 | 0.00 | 0.00 |
| | Day 3 | 0.00 | 0.00 | | Day 3 | 0.00 | 0.00 | | Day 3 | 0.00 | 0.00 |
| | Day 5 | 0.00 | 0.00 | | Day 5 | 0.00 | 0.00 | | Day 5 | 0.00 | 0.01 |
| | Day 7 | 0.01 | 0.00 | | Day 7 | 0.03 | 0.02 | | Day 7 | 0.00 | 0.02 |

### Example 16

Cancellous Bone Granules and Demineralized Cortical Bone Fibers, Incubated with Lyoprotectant Solution, then Pre-Molded into a Strip Shape

For this experiment, the pre-molded strip shape of lyopreserved bone tissue was assessed for cell viability, ease of rehydration, ability to hold shape pre- and post-rehydration, and residual moisture. Two ratios of cancellous granules to cortical fibers were assessed, one ratio containing 40 wt % cancellous granules (Ratio A/RTT Mix) and 60 wt % cortical fibers and the other ratio containing 15% cancellous granules and 85% cortical fibers (Ratio B/FF Mix). Removal of excess protectant solution was assessed for both ratios. Cell viability was assessed by measuring ATP, with results reported below based on the mathematical average of two test samples from each test group derived from a common donor. A control sample that went through the LyoGraft Process was used as a comparison for assessing cell viability.

### A. Protectant Solution Preparation:

1. Protectant solution was prepared by dissolving trehalose and EGCG into 0.9% Saline USP at concentrations of 0.4 M and 2 mM, respectively.
2. Trehalose-saline solution was prepared by dissolving trehalose into 0.9% Saline USP at a concentration of 0.4 M.
3. Following dissolution of preservative ingredients in media, both solutions were sterile filtered using a 0.2 μm membrane filter.

### B. Sample Preparation (Performed in Sterile Hood)

4. Viable cancellous tissue (granules) and demineralized cortical tissue (fibers) were obtained by sterile processing techniques as described in Example 1 above, Part B.1.
5. Bulk cancellous granules and demineralized cortical fibers were packaged under sterile conditions and temporarily stored at refrigerated temperature (2-4° C.) for a period of time greater than 0 and up to 90 minutes, until time of use in experiment.
6. In a sterile hood, cancellous granules were incubated in protectant solution for up to 1 hour.
7. After incubation, cancellous granules were combined with cortical fibers and samples aliquoted for the respective ratios into 2.5 cm×5 cm strip molds.
8. Samples designated with Ratio A included approximately 3.4 g cancellous granules and 5.1 g cortical fibers. Samples designated with Ratio B included approximately 1.1 g cancellous and 6 g cortical fibers
9. 7.19 mL of sterile trehalose-saline solution was added to each mold for tissue with Ratio A. 8.79 mL of sterile trehalose-saline solution was added to each mold for tissue with Ratio B.
10. Molds containing tissue were sealed into Tyvek pouches and placed in the Lyopreserving Apparatus.
11. Tissue was lyopreserved generally according to the following recipes provided below in Table 40 and 41.

TABLE 40
Fiber Strip Cycle

| Fiber Strip Cycle | Temperature | Time | Vacuum | Ramp/ |
|---|---|---|---|---|
| Thermal Treatment | (° C.) | (min) | (mTorr) | Hold |
| Freeze Parameters | 40 | 240 | — | Hold |
| Freeze, Condensor, and | Temperature | Time | Vacuum | Ramp/ |
| Vaccum Parameters | (° C.) | (min) | (mTorr) | Hold |
| Freeze Temperature (° C.) | −40 | | | Hold |
| Additional Freeze (min) | | 270 | | |
| Condensor Set Point (° C.) | −45 | | | |
| Vacuum Set Point (mTorr) | | | 600 | |
| | Temperature | Time | Vacuum | Ramp/ |
| Drying Phase Parameters | (° C.) | (min) | (mTorr) | Hold |
| Step 1 | −40 | 20 | 600 | Hold |
| Secondary Drying Phase | Temperature | Time | Vacuum | Ramp/ |
| Parameters | (° C.) | (min) | (mTorr) | Hold |
| Secondary Set Point | −40 | | | |
| Post Heat Step | −40 | 10 | 600 | Hold |

TABLE 41
Second Cycle

| Second Cycle | Temperature | Time | Vacuum | Ramp/ |
|---|---|---|---|---|
| Thermal Treatment | (° C.) | (min) | (mTorr) | Hold |
| Freeze Parameters | −40 | 180 | — | Hold |
| Freeze, Condensor, and | Temperature | Time | Vacuum | Ramp/ |
| Vaccum Parameters | (° C.) | (min) | (mTorr) | Hold |
| Freeze Temperature (° C.) | −38 | | | Hold |
| Additional Freeze (min) | | 0 | | |
| Condensor Set Point (° C.) | −40 | | | |
| Vacuum Set Point (mTorr) | | | 350 | |
| | Temperature | Time | Vacuum | Ramp/ |
| Drying Phase Parameters | (° C.) | (min) | (mTorr) | Hold |
| Step 1 | 35 | 580 | 350 | Hold |
| Step 2 | | 580 | 350 | Hold |
| Step 3 | | 580 | 350 | Hold |
| Secondary Drying Phase | Temperature | Time | Vacuum | Ramp/ |
| Parameters | (° C.) | (min) | (mTorr) | Hold |
| Secondary Set Point | 23 | | | |
| Post Heat Step | 23 | 60 | 350 | Hold |

### C. Cell Viability Testing

12. Following lyopreservation, samples were placed into jars and rehydrated in 0.9% Saline USP. Solution was added to the fill line of each jar and sat at room temperature for 5 minutes.
13. Following the 5-minute rehydration period, excess solution was decanted from all jars.
14. For each sample, all the tissue (1.3-1.6 g) was tested per the ATP viability assay procedures described above and values corrected based on their pre-lyo weight. Table 42 below captures the results. Results were normalized relative to the RTT Medium Control units and average ATP presented as a percentage.

TABLE 42
ATP results for the different conditions

| Sample | Avg ATP |
|---|---|
| FF Mix, Non-Decanted | 47% |
| FF Mix, Decanted | 45% |
| RTT Mix, Non-Decanted | 59% |
| RTT Mix, Decanted | 62% |
| RTT Medium Control | 100% |

### D. Rehydration and Shape Integrity

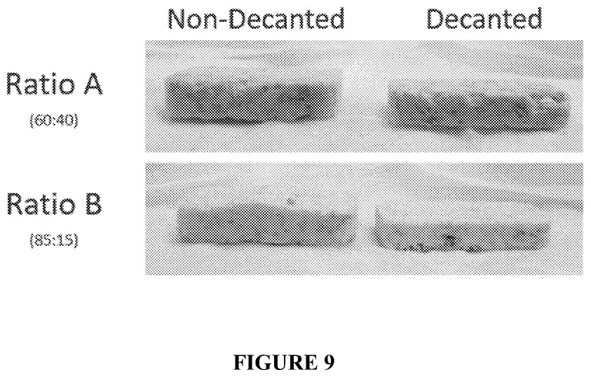

15. Following lyopreservation, the pre-shaped samples were able to maintain the shape of the strip. FIG. 9 shows the results of this evaluation.
16. For rehydrating, all conditions were able to be fully rehydration in under a minute. However, once rehydrated, within seconds, the tissue dissociated and fell apart. Ratio B samples stayed together a few more seconds than Ratio A samples.

### E. Residual Moisture Testing

17. Only residual moisture was assessed through loss-on-drying for decanted and non-decanted tissue with Ratio A. The residual moisture values were 1.8% and 2.2%, respectively.

We claim:

1. A method for repairing, reconstructing, otherwise treating a bone defect, comprising the steps of:
providing a lyopreserved bone tissue form comprising a lyopreserved bone tissue sample which is derived from a recovered tissue sample comprising bone tissue, wherein the lyopreserved bone tissue sample contains one or more populations of endogenous viable cells selected from:
(a) a post-lyopreservation population of endogenous viable cells which is at least 85% of a pre-lyopreservation population of endogenous viable cells of the recovered tissue sample,
(b) a post-lyopreservation population of endogenous viable cells which is at least 85% of a pre-contact population of endogenous viable cells of the recovered tissue sample, and
(c) a retained population of endogenous viable cells which is at least 90% of a post-lyopreservation population of endogenous viable cells, after storage of the lyopreserved bone tissue sample at a temperature above freezing from 14 days to 5 years; and
placing the lyopreserved bone tissue form on or in a subject, at an implantation site which is proximate, adjacent or implanted within the bone defect.

2. The method of claim 1, further comprising rehydrating the lyopreserved bone tissue sample, after the step of providing the lyopreserved bone tissue form and prior to or during, or both prior to and during, the step of placing the lyopreserved bone tissue form, wherein rehydrating is performed by contacting the lyopreserved bone tissue form with a biologically compatible fluid to produce a rehydrated lyopreserved bone tissue form, and placing the rehydrated lyopreserved bone tissue form on or in the subject.

3. The method of claim 2, wherein rehydration is performed prior to placing the lyopreserved bone tissue form, the method further comprises removing excess rehydration fluid and residual lyoprotectant if present in the lyopreserved bone tissue sample, and then placing the lyopreserved bone tissue form.

4. The method of claim 1, wherein repairing, reconstructing, otherwise treating a bone defect comprises orthopedic surgery comprising one or more of: intervertebral spinal fusion, posterolateral spinal fusion, long bone fusion, and fusion of other bones.

5. The method of claim 4, wherein in the step of placing the lyopreserved tissue form on or in a subject comprises implanting the lyopreserved bone tissue sample in an extremity of the subject and wherein the fusion of other bones comprises fusion of an ankle, a foot, a wrist, a hand, or a combination thereof.

6. The method of claim 1, wherein the lyopreserved bone tissue form is not thawed prior to the step of placing.

7. The method of claim 1, wherein the step of providing the lyopreserved bone tissue comprises providing a lyopreserved bone tissue form which further comprises one or more biocompatible fluids and is already at least partially rehydrated prior to the step of placing on or in a subject.

8. The method of claim 1, wherein the lyopreserved bone tissue form further comprises one or more additional components including: mineralized or demineralized bone matrix in the form of chips, particles, fibers, powder, sheets, chunks, pieces, geometric shapes, or a combination thereof; decellularized bone tissue matrix in the form of chips, particles, fibers, powder, sheets, chunks, pieces, geometric shapes, or a combination thereof; and processed or unprocessed cartilage tissue forms.

9. The method of claim 8, wherein the lyopreserved bone tissue sample comprises viable cancellous bone granules, and the lyopreserved bone tissue form further comprises demineralized cortical bone fibers.

10. The method of claim 9, wherein the lyopreserved bone tissue form comprises: from 70% to 30% viable cancellous bone granules, and from 30% to 70% demineralized cortical bone fibers, based on the total weight of the viable cancellous bone granules and demineralized cortical bone fibers.

11. A kit comprising:
a lyopreserved bone tissue form comprising a lyopreserved bone tissue sample which is derived from a recovered tissue sample comprising bone tissue, wherein the lyopreserved bone tissue sample contains one or more populations of endogenous viable cells selected from:
(a) a post-lyopreservation population of endogenous viable cells which is at least 85% of a pre-lyopreservation population of endogenous viable cells of the recovered tissue sample,
(b) a post-lyopreservation population of endogenous viable cells which is at least 85% of a pre-contact population of endogenous viable cells of the recovered tissue sample, and
(c) a retained population of endogenous viable cells which is at least 90% of a post-lyopreservation population of endogenous viable cells, after storage of the lyopreserved bone tissue sample at a temperature above freezing from 14 days to 5 years; and
packaging which includes at least a first container sized and shaped for containing therein at least the lyopreserved bone tissue sample, with or without storage media and with or without one or more biocompatible fluids, the first container comprising a moisture barrier material which prevents or minimizes moisture from entering or escaping from the first container after sealing with the preserved tissue form therein; and
instructions for rehydrating and handling the lyophilized bone tissue sample prior to use.

12. The kit of claim 11, wherein the lyopreserved bone tissue sample and any additional components included in the kit are capable of storage at temperatures above freezing and are all ready for use without thawing.

13. The kit of claim 11, wherein the packaging further includes an inner container sized and shaped for being received and enclosed within the first container and containing therein at least the lyopreserved bone tissue sample.

14. The kit of claim 13, wherein the inner container is permeable for allowing moisture to escape during lyopreservation of a bone tissue sample to produce the lyopreserved bone tissue sample.

15. The kit of claim 13, wherein each of the first container and the inner container comprise, independently of one another, a jar, a vial, a bottle, a pouch, a syringe, a cannula, a tube, or a combination thereof.

16. The kit of claim 15, further comprising at least one biocompatible fluid container which contains one or more biocompatible fluids for combining with the lyopreserved bone tissue sample prior to or during use.

17. The kit of claim 11, wherein the packaging further includes one or more additional component containers, each of which contains one or more additional components.

18. The kit of claim 17, wherein the one or more additional components include: mineralized or demineralized bone matrix in the form of chips, particles, fibers, powder, sheets, chunks, pieces, geometric shapes, or a combination thereof; decellularized bone tissue matrix in the form of chips, particles, fibers, powder, sheets, chunks, pieces, geometric shapes, or a combination thereof; and processed or unprocessed cartilage tissue forms, wherein one or more additional components are contained in the one or more additional component containers.

19. The kit of claim 18, wherein the lyopreserved bone tissue sample comprises viable cancellous bone granules, wherein the lyopreserved bone tissue form further comprises an additional component including demineralized cortical bone fibers contained in at least one additional component container.

20. The kit of claim 19, wherein the lyopreserved bone tissue form comprises: from 70% to 30% viable cancellous bone granules, and from 30% to 70% demineralized cortical bone fibers, based on the total weight of the viable cancellous bone granules and demineralized cortical bone fibers.
